SEQUENCE LISTING (39 sequences)

SEQ ID NO: 1 - protein, 125 aa, synthetic construct
LMENYPGTLQALGEPIRQGHFIVWEGAPGARMPWKGHNRHVFLFRNHLVICKPRRDSRTDTVSYVFRNMMKLSSIDLNDQVEGDDRAFEVWQEREDSVRKYLLQARTAIIKSSWVKEICGIQQRL

SEQ ID NO: 2 - DNA, 375 bp, synthetic construct
  source 1..375: mol_type = other DNA
ctcatggagaactacccaggcaccctgcaggccctgggcgagcccatccgccagggccacttcatcgtgtgggagggtgcaccgggggcccgcatgccctggaagggccacaaccgtcacgtgttcctcttccgcaaccacctggtaatctgcaagccccggcgagactcccgcaccgataccgtcagctacgtgttccggaacatgatgaagctgagcagcatcgacctgaacgaccaggtggagggggatgaccgcgccttcgaggtgtggcaggagcgggaggactcggtgcgcaagtacctgctgcaggcacggacagccattatcaagagctcgtgggtgaaggagatctgtggcatccagcagcgtctg

SEQ ID NO: 3 - protein, 123 aa, synthetic construct
MLEGFDENLDVQGELILQDAFQVWDPKSLIRKGRERHLFLFEISLVFSKEIKDSSGHTKYVYKNKLLTSELGVTEHVEGDPCKFALWSGRTPSSDNKTVLKASNIETKQEWIKNIREVIQERI

SEQ ID NO: 4 - DNA, 369 bp, synthetic construct
  source 1..369: mol_type = other DNA
atgctggaagggttcgacgagaacctggatgtgcagggggagttgattctccaggatgcctttcaagtgtgggacccgaagtcgctgatccggaaggggcgggagcggcacttgttcctctttgagatctccttggtttttagcaaggagatcaaagattcttcaggacacacgaaatatgtttacaagaacaagctactgacctcagagctgggtgtgaccgagcacgtggagggcgatccctgcaaattcgccttgtggtctgggcgcaccccatcctcagacaataaaacagtgctgaaagcctccaacattgaaaccaagcaggagtggatcaagaacattcgagaagtgattcaagaaaggatc

SEQ ID NO: 5 - protein, 123 aa, synthetic construct
LEVGTVMTLFYSKKSQRPERKTFQVKLETRQITWSRGADKIEGAIDIREIKEIRPGKTSRDFDRYQEDPAFRPDQSHCFVILYGMEFRLKTLSLQATSEDEVNMWIKGLTWLMEDTLQAPTPL

SEQ ID NO: 6 - DNA, 370 bp, synthetic construct
  source 1..370: mol_type = other DNA
ctcgaggtgggcaccgtcatgactttgttctactccaagaagtcgcagcgacccgagcggaagaccttccaggtcaagctggagacgcgccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgc

SEQ ID NO: 7 - protein, 9 aa, synthetic construct
MGSSKSKPK

SEQ ID NO: 8 - DNA, 27 bp, synthetic construct
  source 1..27: mol_type = other DNA
atgggaagcagcaagagcaagccaaag

SEQ ID NO: 9 - DNA, 34 bp, synthetic construct
  source 1..34: mol_type = other DNA
cgcgccatgggaagcagcaagagcaagccaaagc

SEQ ID NO: 10 - DNA, 34 bp, synthetic construct
  source 1..34: mol_type = other DNA
tcgagctttggcttgctcttgctgcttcccatgg

SEQ ID NO: 11 - DNA, 41 bp, synthetic construct
  source 1..41: mol_type = other DNA
taagcaggcgcgccatgggaagcagcaagagcaagccaaag

SEQ ID NO: 12 - DNA, 47 bp, synthetic construct
  source 1..47: mol_type = other DNA
tgcttagtttaaacttacagatcctcttctgagatgagtttctgctc

SEQ ID NO: 13 - DNA, 41 bp, synthetic construct
  source 1..41: mol_type = other DNA
taagcaggcgcgccatgctcatggagaactacccaggcacc

SEQ ID NO: 14 - DNA, 33 bp, synthetic construct
  source 1..33: mol_type = other DNA
tgcttactcgagcagacgctgctggatgccaca

SEQ ID NO: 15 - DNA, 41 bp, synthetic construct; identical to SEQ ID NO: 13
  source 1..41: mol_type = other DNA

SEQ ID NO: 16 - DNA, 47 bp, synthetic construct; identical to SEQ ID NO: 12
  source 1..47: mol_type = other DNA

SEQ ID NO: 17 - DNA, 44 bp, synthetic construct
  source 1..44: mol_type = other DNA
gcagcaagagcaagccaaagctcatggagaactacccaggcacc

SEQ ID NO: 18 - DNA, 41 bp, synthetic construct; identical to SEQ ID NO: 11
  source 1..41: mol_type = other DNA

SEQ ID NO: 19 - DNA, 47 bp, synthetic construct; identical to SEQ ID NO: 12
  source 1..47: mol_type = other DNA

SEQ ID NO: 20 - DNA, 38 bp, synthetic construct
  source 1..38: mol_type = other DNA
ctagatggcgcgccatgctggaaggctttgatgaaaac

SEQ ID NO: 21 - DNA, 36 bp, synthetic construct
  source 1..36: mol_type = other DNA
tagcctctcgagaatgcgttcctgaatcacttcgcg

SEQ ID NO: 22 - DNA, 38 bp, synthetic construct; identical to SEQ ID NO: 20
  source 1..38: mol_type = other DNA

SEQ ID NO: 23 - DNA, 47 bp, synthetic construct
  source 1..47: mol_type = other DNA
tgctcagtttaaacttacagatcctcttctgagatgagtttctgctc

SEQ ID NO: 24 - DNA, 44 bp, synthetic construct
  source 1..44: mol_type = other DNA
gcagcaagagcaagccaaagatgctggaaggctttgatgaaaac

SEQ ID NO: 25 - DNA, 41 bp, synthetic construct; identical to SEQ ID NO: 11
  source 1..41: mol_type = other DNA

SEQ ID NO: 26 - DNA, 47 bp, synthetic construct; identical to SEQ ID NO: 23
  source 1..47: mol_type = other DNA

SEQ ID NO: 27 - DNA, 38 bp, synthetic construct
  source 1..38: mol_type = other DNA
ctagatggcgcgccatgctggaagtgggcaccgtgatg

SEQ ID NO: 28 - DNA, 47 bp, synthetic construct; identical to SEQ ID NO: 23
  source 1..47: mol_type = other DNA

SEQ ID NO: 29 - DNA, 41 bp, synthetic construct; identical to SEQ ID NO: 11
  source 1..41: mol_type = other DNA

SEQ ID NO: 30 - DNA, 47 bp, synthetic construct; identical to SEQ ID NO: 23
  source 1..47: mol_type = other DNA

SEQ ID NO: 31 - DNA, 20 bp, synthetic construct
  source 1..20: mol_type = other DNA
caagccaaagctcgagcaga

SEQ ID NO: 32 - DNA, 20 bp, synthetic construct
  source 1..20: mol_type = other DNA
cttgccccttgctccatacc

SEQ ID NO: 33 - DNA, 20 bp, synthetic construct
  source 1..20: mol_type = other DNA
agcaagccaaagctcatgga

SEQ ID NO: 34 - DNA, 20 bp, synthetic construct
  source 1..20: mol_type = other DNA
agattaccaggtggttgcgg

SEQ ID NO: 35 - DNA, 21 bp, synthetic construct
  source 1..21: mol_type = other DNA
tcactcaaagccgctcaacta

SEQ ID NO: 36 - DNA, 24 bp, synthetic construct
  source 1..24: mol_type = other DNA
tctgccttcatttcgttatgtacc

SEQ ID NO: 37 - DNA, 18 bp, synthetic construct
  source 1..18: mol_type = other DNA
tcggagtcaacggatttg

SEQ ID NO: 38 - DNA, 23 bp, synthetic construct
  source 1..23: mol_type = other DNA
caacaatatccactttaccagag

SEQ ID NO: 39 - DNA, 27 bp, synthetic construct
  source 1..27: mol_type = other DNA
atgggaagcagcaagagcaagccaaag

(19) United States
(12) Patent Application Publication
(10) Pub. No.: US 2026/0209295 A1
(43) Pub. Date: Jul. 23, 2026

(54) COMPOSITIONS AND METHODS FOR TREATING CANCER METASTASIS BY ADMINISTERING NUCLEIC ACIDS ENCODING PLECKSTRIN HOMOLOGY DOMAINS

(71) Applicants: UNIVERSITY OF MARYLAND, BALTIMORE, Baltimore, MD (US); The Johns Hopkins University, Baltimore, MD (US)

(72) Inventors: Aikaterini Kontrogianni-Konstantopoulos, Ellicott City, MD (US); Matthew Kent Eason, Baltimore, MD (US); Konstantinos Konstantopoulos, Ellicott City, MD (US)

(21) Appl. No.: 19/454,051

(22) Filed: Jan. 20, 2026

Related U.S. Application Data
(60) Provisional application No. 63/746,479, filed on Jan. 17, 2025.

(51) Int. Cl. C07K 14/47; A61K 9/1271; A61K 9/51; A61K 31/711; A61K 38/00; A61P 35/04; C12N 9/20; C12N 15/86; C12N 15/88

(52) U.S. Cl. CPC C07K 14/4705; A61K 9/1271; A61K 9/5123; A61K 31/711; A61P 35/04; C12N 9/20; C12N 15/86; C12N 15/88; A61K 38/00; C12N 2710/10043; C12N 2740/10043; C12N 2740/15043; C12N 2750/14143; C12Y 301/01004

(57) ABSTRACT

The present invention provides a pharmaceutical composition comprising an effective amount of nucleic acid encoding a polypeptide comprising a pleckstrin homology (PH) domain, a variant or fragment thereof, wherein the fragment of the PH domain comprises a p85-SH3 interacting region (PSIR), and methods of treating cancer comprising administering the pharmaceutical composition to subjects.

### CROSS-REFERENCE TO RELATED APPLICATIONS

This application claims the benefit of U.S. Application No. 63/746,479, filed Jan. 17, 2025, the contents of which is incorporated by reference in their entirety.

### STATEMENT REGARDING FEDERALLY SPONSORED RESEARCH AND DEVELOPMENT

This invention was made with government support under the Grant Numbers CA183804, CA278384, and CA154274 awarded by the National Institutes of Health. The government has certain rights in the invention.”

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 37,310 Byte XML file named “Sequence_listing.xml,” created on Jan. 20, 2026.

### FIELD OF THE INVENTION

The present invention generally relates to the fields of molecular biology, medicine and cancer. In particular, the field of the invention relates to compositions and methods for the treatment of breast cancer metastasis.

### BACKGROUND OF THE INVENTION

Obscurin (720-870 kDa) is a giant cytoskeletal protein encoded by the single OBSCN gene (T. Guardia et al., Biochim Biophys Acta Rev Cancer, (2021), 1876:188567, https://doi.org/10.1016/j.bbcan.2021.188567; A. Kontrogianni-Konstantopoulos et al., Physiol Rev, (2009), 89:1217). OBSCN loss has been implicated in breast cancer formation and progression [1], as well as in chemotherapy and radiotherapy resistance (T. Guardia et al., Biochim Biophys Acta Rev Cancer, (2021), 1876:188567; N. A. Perry et al., FASEB J, (2012), 26:2764; T. Guardia et al., Proc Natl Acad Sci USA, (2023), 120:e2215553120; M. Shriver et al., Oncotarget, (2016), 7:45414; N. A. Perry et al., Oncotarget, (2014), 5:8558; B. K. Rajendran et al., Oncotarget, (2017), 8:102263; M. Shriver et al., Oncogene, (2015), 34: 4248; L. Yang et al., Int J Radiat Oncol Biol Phys, (2020), 108:1103). Accordingly, analysis of Kaplan-Meier datasets indicates that low OBSCN levels correlate with significantly reduced survival and relapse-free survival in breast cancer patients (T. Guardia et al., Proc Natl Acad Sci USA, (2023), 120:e2215553120). Conversely, higher OBSCN levels correlate with increased patient responsiveness to anthracyclines, commonly used in metastatic breast cancer treatment (T. Guardia et al., Proc Natl Acad Sci USA, (2023), 120:e2215553120). Consistent with these observations, obscurin is abundantly expressed in normal breast epithelium, where it preferentially concentrates at the cell membrane and perinuclearly at the Golgi, but is dramatically diminished in advanced stage breast cancer biopsies and cell lines (N. A. Perry et al., FASEB J, (2012), 26:2764; T. Guardia et al., Proc Natl Acad Sci USA, (2023), 120:e2215553120; M. Shriver et al., Oncotarget, (2016), 7:45414; N. A. Perry et al., Oncotarget, (2014), 5:855; M. Shriver et al., Oncogene, (2015), 34: 4248). Knockdown of obscurin in normal breast epithelium induces epithelial to mesenchymal transition (EMT) and stemness, reduces cell sensitivity to common chemotherapies (e.g., paclitaxel), enhances migration and invasion, and promotes metastasis (N. A. Perry et al., Oncotarget, (2014), 5:8558; M. Shriver et al., Oncogene, (2015), 34: 4248). Mechanistically, loss of obscurin results in upregulation of the PI3K/Akt axis, which is altered in 30-40% of invasive breast carcinomas, driving breast cancer cell growth and metastatic dissemination via a two-tiered system of gene expression and cytoskeletal changes (M. Shriver et al., Oncotarget, (2016), 7:45414; Y. He et al., Signal Transduct Target Ther, (2021), 6:425; D. Miricescu et al., Int J Mol Sci, (2020), 22 (1); X. Li et al., Int J Oncol, (2013), 43:793; W. B. Huh et al., PLoS One, (2015), 10:e0128365; C. Jimenez et al., J Cell Biol, (2000), 151:249; S. Corallino et al., Nat Commun, (2018), 9:1475; D. Hoshino et al., Sci Signal, (2012), 5:ra66). Obscurin forms a complex with the PI3K-p85 regulatory subunit (M. Shriver et al., Oncotarget, (2016), 7:45414). Their interaction is direct and mediated by the obscurin-Pleckstrin Homology (PH) domain and the PI3K-p85-Src Homology 3 (SH3) domain with a K_{D} of ~50 nM (M. Shriver et al., Oncotarget, (2016), 7:45414).

What is needed are new and effective therapies to treat or prevent cancer metastasis, including breast cancer metastasis. This background information is provided for informational purposes only. No admission is necessarily intended, nor should it be construed, that any of the preceding information constitutes prior art against the present disclosure.

### SUMMARY OF THE INVENTION

It is to be understood that both the foregoing general description of the embodiments and the following detailed description are exemplary, and thus do not restrict the scope of the embodiments.

The present disclosure describes an investigation of the potential suppressive properties of a mini-obscurin, consisting of the obscurin-PH domain—which is ~50 times smaller than the full-length protein—in two of the most aggressive breast cancer subtypes, i.e., triple negative and HER2+. This disclosure demonstrates that obscurin-PH is a potent PI3K inhibitor with anti-growth and anti-metastatic capabilities and further uncover a new class of PI3K inhibitors, in the form of a subgroup of structurally homologous PH-domains, uniquely targeting the p85 regulatory subunit.

Importantly, this disclosure is distinct from prior and ongoing strategies that focus on the development of small molecule PI3K inhibitors exclusively targeting the p110 catalytic subunit, which however exhibit dose-limiting toxicity and are FDA approved solely for metastatic estrogen receptor-positive breast cancer that has failed systemic hormone therapy (Y. He et al., Signal Transduct Target Ther, (2021), 6:425). In sum, this disclosure illustrates the therapeutic potential of a new class of non-chemical PI3K inhibitors in the form of the PH-domain, which can be used as nanoparticle-delivered local gene therapy for the treatment of metastatic breast cancer beyond the hormone receptor-positive subtype.

In one aspect, the disclosure provides a pharmaceutical composition comprising an effective amount of nucleic acid encoding a polypeptide comprising a pleckstrin homology (PH) domain, a variant or fragment thereof, wherein the fragment of the PH domain comprises a p85-SH3 interacting region (PSIR).

In some embodiments, the nucleic acid is RNA. In some embodiments, the nucleic acid is DNA. In some embodiments, the nucleic acid encodes a polypeptide that interacts with the PI3K-p85 regulatory subunit.

In some embodiments, the polypeptide comprises a myristoylation tag. In some embodiments, the myristoylation tag is located at the N-terminus of the polypeptide. In some embodiments, the myristoylation tag comprises an amino acid sequence of Met-Gly-Xaa-Xaa-Xaa-Ser/Thr-Xaa-Xaa. In some embodiments, the myristoylation tag comprises an amino acid sequence comprising Met-Gly-Ser-Ser-Lys-Ser-Lys-Pro-Lys (SEQ ID NO:7). In some embodiments, the myristoylation tag comprises is encoded by a nucleotide sequence comprising ATGGGAAGCAGCAAGAGCAAGCCAAAG (SEQ ID NO:39).

In some embodiments, the polypeptide consists essentially of a myristoylation tag and a pleckstrin homology (PH) domain, a variant or fragment thereof, wherein the fragment of the PH domain comprises a p85-SH3 interacting region (PSIR).

In some embodiments, the pleckstrin homology (PH) domain is from a human protein. In some embodiments, the PH domain comprises any one of SEQ ID NOS: 1, 3 or 5. In some embodiments, pleckstrin homology (PH) domain is at least 90% identical to any one of SEQ ID NOS: 1, 3 or 5. In some embodiments, the pleckstrin homology (PH) domain is from an obscurin protein. In some embodiments, the pleckstrin homology (PH) domain is from a kalirin protein. In some embodiments, the pleckstrin homology (PH) domain is from a phospholipase-Cγ1 (PLCγ1).

In some embodiments, the nucleic acid is encoded by a viral vector. In some embodiments, the viral vector is selected from an adeno-associated viral vector, an adenoviral vector, a lentiviral vector, and a retroviral vector.

In some embodiments, the composition comprises lipid nanoparticles that encapsulate the nucleic acid. In some embodiments, the lipid nanoparticles comprise Dlin-KC2-DMA (2,2-dilinoleyl-4-dimethylaminoethyl-[1,3]-dioxolane), DSPC (1,2-distearoyl-sn-glycero-3-phosphocholine), cholesterol, and mPEG2000-DSPE (1,2-dimyristoyl-rac-glycero-3-methoxypolyethyleneglycol-2000. In some embodiments, the molar ratio of KC2:DSPC:cholesterol:mPEG2000-DSPE ranges from 30-50:5-15:25-40:0.5-5. In some embodiments, the molar ratio of KC2:DSPC:cholesterol:mPEG2000-DSPE is about 50:10.5:38.1:1.5. In some embodiments, the lipid nanoparticles have a nitrogen-to-phosphate ratio (N/P) of between 5-10. In some embodiments, the lipid nanoparticles have a nitrogen-to-phosphate ratio (N/P) of about 8.

In another aspect, the disclosure provides a method of treating cancer in a subject, comprising administering to the subject an effective amount of the pharmaceutical composition herein. In some embodiments, the cancer is breast cancer. In some embodiments, the breast cancer is metastatic breast cancer.

In some embodiments, the administering suppresses PI3K/Akt activity in cancer cells as evidenced by reduced phosphorylation levels of activating p85-Tyr458 as well as Akt-Thr308 and -Ser473.

In some embodiments, the administering inhibits formation of filopodia in cancer cells.

In some embodiments, the administering inhibits migration and adhesion of cancer cells to pre-metastatic niche extracellular matrix substrates.

In some embodiments, the administering inhibits invadopodia and matrix metalloproteinase expression.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

FIG. 1. Sequestration of PI3K-p85 by the obscurin PH-domain suppresses PI3K/Akt activation and reduces breast cancer cell migration and adhesion. (A) Representative confocal images of adenovirus-transduced MDA-MB-231 and SKBr3 cells expressing the Myr-Myc and Myr-oPH-Myc constructs; DAPI (blue), Myc-Tag (green), PI3K-p85 (orange), and merged Myc-tag/PI3K-p85 (yellow). Cells of each treatment group vary significantly in size; scale bars differ to visualize the entire cell. Line composite graphs display PI3K-p85 and Myc-tag gray values (pixel intensity). (B-C) Immunoblots of MDA-MB-231 (B) and SKBr3 (C) cells expressing the Myr-Myc and Myr-oPH-Myc constructs probed for PI3K-p85 Y458 and pAkt T308/S473 phosphorylation levels. Densitometry values are presented as expression relative to the Myr-Myc control, set at “1” (n=3 independent experiments); two-tailed t-test. (D) Representative images of MDA-MB-231 single cells (yellow arrowheads), expressing the Myr-Myc or Myr-oPH-Myc construct, migrating through microchannels. (E-I) Percent (%) cell entry into microchannels (e), mean cell entry time (min; F), single cell speed (μm/h; G), velocity (μm/h; H), and persistence (I) of MDA-MB-231 cells expressing the Myr-Myc or Myr-oPH-Myc constructs (n=88-92 single cells per construct pooled from 3 independent experiments); two-tailed t-test (E and F) and Mann-Whitney test (G-I). (J-K) Representative wide-field fluorescent images of Myr-Myc or Myr-oPH-Myc expressing MDA-MB-231 (J) and SKBr3 (K) cells labelled with DAPI (blue), following adhesion to plastic, fibronectin, collagen I, or laminin. Average DAPI-stained cells/well are plotted (n=4-6 independent experiments per condition); two-way ANOVA with Sidak's multiple comparison test. All data are plotted as mean±SD; *p<0.05; **p<0.01; ***p<0.001; ****p<0.0001.

FIG. 2. Filopodia ablation by the obscurin PH-domain inhibits breast cancer cell migration and adhesion. (A) Representative confocal images of adenovirus-transduced, RFP-positive MDA-MB-231 cells expressing the Myr-Myc or Myr-oPH-Myc construct plated on plastic, fibronectin, collagen I, or laminin substrates; RFP (orange), Myc-tag (green), β-actin (magenta), DAPI (blue), and filopodia FiloQuant Overlay mask (white). Cells and filopodia of each treatment group vary significantly in size; scale bars differ to visualize the entire cell and their filopodia. Average number and length (μm) of filopodia (FiloQuant) are plotted per cell, per extracellular matrix substrate (n=50 cells per condition pooled from 5 independent experiments). (B), Representative confocal images of adenovirus-transduced, RFP-positive MDA-MB-231 cells expressing the Myr-Myc or Myr-oPH-Myc construct plated on collagen I, treated with vehicle control (VC) or 100 ng/mL bradykinin (BK); RFP (orange), Myc-tag (green), β-actin (magenta), DAPI (blue), and filopodia FiloQuant Overlay mask (white). Cells and filopodia of each treatment group vary significantly in size; scale bars differ to visualize the entire cell and their filopodia. Average number and length (μm) of filopodia (FiloQuant) are plotted per cell, per treatment group (n=30 cells per treatment pooled from 3 independent experiments). (C) Representative brightfield images of transwell migration assays of MDA-MB-231 cells expressing the Myr-Myc or Myr-oPH-Myc construct, treated with vehicle control (VC) or 100 ng/mL bradykinin (BK). The percent (%) migrated cells per field are plotted, per treatment group (n=3 independent experiments). (D) Representative images of Myr-Myc and Myr-oPH-Myc expressing MDA-MB-231 cells stained with DAPI (blue) to allow visualization of nuclei, treated with vehicle control (VC) or 100 ng/mL bradykinin (BK), following adhesion to collagen I. Average DAPI-labeled cells/well are plotted (n=6 independent experiments). All statistical tests were performed with two-way ANOVA followed by Tukey's multiple comparison test. All data are plotted as mean±SD; *p<0.05; **p<0.01; ***p<0.001; ****p<0.0001.

FIG. 3. Ectopic expression of the obscurin PH-domain silences breast cancer matrix metalloproteinase (MMPs) expression and invadopodia formation to inhibit invasion. (A-C) cBioPortal Illumina HT-12 v3 microarray (Breast Cancer METABRIC, Nature 2012 & Nat Commun 2016) MMP1 expression for all breast carcinoma subtypes (n=541 patient samples per group; A), MMP2 expression for claudin-low subtype breast cancer (n=109 patient samples per group; B), and MMP9 expression for invasive lobular carcinoma (ILC) subtype breast cancer (n=73 patient samples per group; C), separated by low and high OBSCNmRNA expression (median); p-values by two-tailed t-test; q-values by Benjamini-Hochberg procedure. (D-F) Kaplan Meier Plotter analysis (http://kmplot.com/analysis/index.php?p=service) of overall survival via low and high OBSCN:MMP expression ratio for (D) all breast cancer subtypes by OBSCN:MMP1, (E) ER−/PR−/HER2− by OBSCN:MMP2, and (F) HER2 by OBSCN:MMP9; Log Rank test. (G-H) Western blot of Myr-Myc or Myr-oPH-Myc expressing MDA-MB-231 (G) and SKBr3 (H) cells for MMP1, MMP2, and MMP9 expression; (n=3 independent experiments), two-tailed t-test. (I-J) Representative confocal images of gelatin invadopodia assays for Myr-Myc or Myr-oPH-Myc transduced MDA-MB-231 (I) and SKBr3 (J) cells; Gelatin (red), RFP (gold), Myc-tag (Cyan), F-actin (green), and MMP-2 (MDA-MB-231; magenta) and MMP-9 (SKBr3; magenta). Zoomed images include borders of invadopodia (black dotted lines), MMP-positive invadopodia (blue arrowheads), MMP-positive puncta (white arrowheads). Line composite graphs display F-actin, MMP-2 (MDA-MB-231), MMP-9 (SKBr3), and Gelatin Mean Gray Values; cell regions with invadopodia are denoted by grey boxes. Gelatin degradation/cell and invadopodia abundance (calculated as the complement Gelatin/F-actin Mander's coefficient relative to Myr-Myc) are plotted; (n=30 cells per construct pooled from 3 independent experiments); Mann-Whitney test. Data are plotted as mean±SD; *p<0.05; **p<0.01; ***p<0.001; ****p<0.0001.

FIG. 4. Ectopic expression of the obscurin PH-domain halts breast cancer dissemination from spheroids. (A) Representative images of MDA-MB-231 Myr-Myc or Myr-oPH-Myc expressing spheroids 37.5 h following embedding into 3D collagen I. Red frames indicate the spheroid edge at t=0 h; red arrowheads denote invasion edges at t=37.5 h. (B-C) Initial spheroid area (B) at t=0 h and spheroid area of expansion (C) at t=18.33 h for Myr-Myc (n=48 spheroids) and Myr-oPH-Myc (n=57 spheroids), pooled from 3 independent experiments; Mann-Whitney test. (D) Representative images of single dissociated cells (yellow arrowheads) from Myr-Myc or Myr-oPH-Myc expressing spheroids during a total elapsed time of 3.33 h. (E-H) Spheroid dissociated single cell speed (μm/h; E), velocity (μm/h; F), mean square displacement (MSD; μm²×10³) over 4 h (G), and trajectories (H) for Myr-Myc (n=54 cells) and Myr-oPH-Myc (74 cells) spheroids, each pooled from 3 independent experiments; Mann-Whitney test (E and F); two-way ANOVA (G); Data are plotted as mean±SD (B, C, E and F) or mean±SEM (G); ****p<0.0001.

FIG. 5. Lipid nanoparticle delivery of the obscurin PH-domain suppresses breast cancer growth and inhibits metastasis. (A) Experimental mouse model schematic detailing the in vivo LNP treatment of orthotopic MDA-MB-231 tumors. 1×10⁶ MDA-MB-231 cells were injected into the mammary fat pad of female NOD SCID mice on Day 0 and were allowed to reach a volume of ~100 mm³, at which point Myr-Myc or Myr-oPH-Myc LNPs were administered intratumorally once a week for 6 weeks. Endpoint was specified as the point at which the primary tumor volume reached 1.5 cm³, followed by harvesting of the primary tumor, along with common metastatic organs, including the lungs, liver, bone, axillary lymph node, and brain; schematic created with Biorender.com (agreement number UW289RJIVN). (B) Caliper measurements of MDA-MB-231 tumor volumes throughout a 6-week course of intratumoral injection of Myr-Myc or Myr-oPH-Myc LNPs (n=9 mice per group pooled from 2 independent experiments); two-way ANOVA. (C) MDA-MB-231 primary tumor masses (in grams) at harvesting, following a 6-week course of intratumoral injection of Myr-Myc or Myr-oPH-Myc LNPs (n=9 mice per group pooled from 2 independent experiments); two-tailed t-test. (D) Immunoblots of Myr-Myc or Myr-oPH-Myc LNP-treated MDA-MB-231 tumors for PI3K-p85 Y458 and pAkt T308/5473 phosphorylation levels. Densitometry values are presented as expression relative to Myr-Myc control (n=9 mice per group pooled from 2 independent experiments); two-tailed t-test. (E-H) qPCR of lungs (E), liver (F), bone (G), and brain (H) for human Long Interspersed Nuclear Element (hLINE) DNA from Myr-Myc or Myr-oPH-Myc treated mice. hLINE DNA is plotted as μg hLINE DNA per mg tissue section (n=9 mice per group pooled from 2 independent experiments); Mann-Whitney test. (I-J) Representative hematoxylin and eosin (H&E; I) and anti-human mitochondria (J) staining of lungs from Myr-Myc or Myr-oPH-Myc treated mice. (K-L) Representative H&E (K) and anti-human mitochondria (L) staining of liver from Myr-Myc or Myr-oPH-Myc treated mice revealing MDA-MB-231 tumor emboli lodged within portal sinusoids (red asterisks) in the control but not the experimental group. (M) Immunoblots of Myr-Myc or Myr-oPH-Myc LNP-treated MDA-MB-231 tumors for MMP1, Pro-MMP2, MMP2, Pro-MMP9 and MMP9. Densitometry values are presented relative to Myr-Myc control (n=9 mice per group pooled from 2 independent experiments); Mann-Whitney test (MMP1) and two-tailed t-test (Pro-MMP2, MMP2, Pro-MMP9, and MMP9). Data are plotted as mean±SD (C) or mean SEM (B, D-H and M); *p<0.05; **p<0.01; ***p<0.001; ****p<0.0001.

FIG. 6. Sequestration of PI3K-p85 by the Kalirin and PLC γ1 PH-domains diminishes PI3K/Akt activation to block breast cancer dissemination. (A) Representative images from STED super resolution microscopy of MDA-MB-231 cells transiently transfected with the Myr-Myc, Myr-oPH-Myc, Myr-kPH-Myc, or Myr-yPH-Myc pDNA constructs. Single MDA-MB-231 cells immunostained for Myc-tag (green) and PI3K-p85 (magenta) are shown at 60×, while zoom-in areas at the cell periphery marked in yellow are shown at 600×, with areas of colocalization between Myc-tag and PI3K-p85 appearing white and PI3K-p85 cytoplasmic puncta that do not colocalize with Myc-Tag denoted with yellow arrowheads. (B) Plotted quantification of average PI3K-p85 sequestration in MDA-MB-231 cells expressing Myr-Myc, Myr-oPH-Myc, Myr-kPH-Myc, or Myr-yPH-Myc constructs (n=15 cells per construct pooled from 3 independent experiments). (C) Western blots of MDA-MB-231 cells transiently transfected with the Myr-Myc, Myr-oPH-Myc, Myr-kPH-Myc, or Myr-yPH-Myc constructs, 2 h post-serum stimulus, probed for PI3K-p85 Y458 and pAkt T308/5473 phosphorylation levels followed by densitometric evaluation (n=7 independent experiments); Kruskal-Wallis test with Dunn's multiple comparison test (pp85-Y458, pAkt-S473) and one-way ANOVA with Dunnett' multiple comparison test (pAkt-T308, total p85, total Akt). (D) Representative brightfield images of transwell migration assays of MDA-MB-231 cells transiently transfected with Myr-Myc, Myr-oPH-Myc, Myr-kPH-Myc, or Myr-yPH-Myc constructs. The percent (%) migrated cells per field are plotted per treatment group (n=4 independent experiments); one-way ANOVA with Tukey's multiple comparison test. (E) Representative images of MDA-MB-231 spheroids treated with LNPs delivering the Myr-Myc, Myr-oPH-Myc, Myr-kPH-Myc, or Myr-yPH-Myc constructs 14 h following embedding into 3D collagen I. Red frames indicate the spheroid edge at t=0 h, while red arrowheads pinpoint invasion edges at t=14 h. (F) Ratio of spheroid area of expansion from t=0 to t=14 h (n=40-47 spheroids per construct, pooled from 3 independent experiments); Kruskal-Wallis test with Dunn's multiple comparison test. (G-J) Spheroid dissociated single cell speed (μm/h; G), velocity (μm/h; H), mean square displacement (MSD; μm²) over 8.33 h (I), and trajectories (J); n=95 randomly selected spheroid single cells per construct pooled from 3 independent experiments (G, H, and J); n=117-248 spheroid single cells per construct pooled from 3 independent experiments (I); Kruskal-Wallis test with Dunn's multiple comparison test (G-H); two-way ANOVA followed by Tukey's multiple comparison test (I). (K) Representative confocal images of Myr-Myc, Myr-oPH-Myc, Myr-kPH-Myc, or Myr-yPH-Myc LNP-treated spheroids stained for MMP2 (red), F-actin (magenta), DAPI (blue) and Myc-tag (green). MMP-2 mean gray values per spheroid are plotted (n=5-10 spheroids); one-way ANOVA with Tukey's multiple comparison test; Data are plotted as mean±SD (B-D, F-H, and K) or mean±SEM (I); *p<0.05; **p<0.01; ***p<0.001 ****p<0.0001.

FIG. 7. The obscurin PH-domain hinders breast cancer collective and chemotactic cell migration. (A) Schematic of myristoylated obscurin pleckstrin homology (PH)-domain with C-terminal Myc-Tag (Myr-oPH-Myc); created with Biorender.com (agreement number LL289RLO7B). (B) Representative Western blot image of MCF10A, MDA-MB-231, and SKBR3 cells probed for obscurin expression; Hsp90 was used as loading control. Densitometry values are presented as expression relative to MCF10A, which was set to “1” (n=3 independent experiments); One-Way ANOVA with Tukey's multiple comparison test. (C-D) Representative phase contrast images of wound healing assays of adenovirus-transduced MDA-MB-231 (C) and SKBr3 (D) cells expressing the Myr-Myc and Myr-oPH-Myc constructs at 0 h and 24 h (MDA-MB-231) or 45 h (SKBr3) following scratch. Wound boarders are outlined in yellow. Percent (%) wound closure is plotted for the 16, 24, and 42 h time point (MDA-MB-231) or 24, 45, and 96 h time points (SKBr3) following scratch (n=5-6 independent experiments); two-tailed t-test. (E-F) Representative brightfield images of transwell migration assays of adenovirus-transduced MDA-MB-231 (E) and SKBr3 (F) cells expressing the Myr-Myc and Myr-oPH-Myc constructs. The percent (%) migrated cells per field are plotted, per treatment group (n=3 independent experiments); two-tailed t-test. All data are plotted as mean±SD; *p<0.05; **p<0.01; ***p<0.001; ****p<0.0001.

FIG. 8. The obscurin PH-domain ablates HER2+ breast cancer cell filopodia. Representative confocal images of adenovirus-transduced, RFP-positive SKBr3 cells expressing the Myr-Myc or Myr-oPH-Myc construct plated on plastic, fibronectin, collagen I, or laminin substrates; RFP (orange), Myc-tag (green), β-actin (magenta), DAPI (blue), and filopodia FiloQuant Overlay mask (white). Cells and filopodia of each treatment group vary significantly in size; scale bars differ to visualize the entire cell and their filopodia. Average number and length (μm) of filopodia (FiloQuant) are plotted per cell, per extracellular matrix substrate (n=50 cells per condition pooled from 5 independent experiments). All statistical tests are Two-Way ANOVA with Tukey's multiple comparison test. All data are plotted as mean±SD; **p<0.01; ****p<0.0001.

FIG. 9. The Obscurin PH-domain silences matrix metalloproteinase (MMPs) expression. (A-B) Illumina HiSeq MMP1 (A) and MMP9 (B) RNAseq expression analysis of normal breast (n=113 patient samples) and invasive breast carcinoma (n=1052 patient samples); Wilcoxon Rank Sum test. (C) cBioPortal Illumina HT-12 v3 microarray MMP2 Expression (Breast Cancer METABRIC, Nature 2012 & Nat Commun 2016) for breast carcinoma, partitioned by the PAM50 subtype classification as either normal (n=148 patient samples) or claudin-low (n=218 patient samples) breast cancer; p-values by two-tailed t-test; q-values by Benjamini-Hochberg procedure (D-E) Representative confocal images of adenovirus-transduced, RFP-positive MDA-MB-231 (D) and SKBr3 (E) cells expressing the Myr-Myc or Myr-oPH-Myc construct plated on gelatin; RFP (red), Myc-tag (blue), MMP2 (D) or MMP9 (E) (green), and β-actin (magenta). MMP2 (D) or MMP9 (E) mean gray values (pixel intensity) are plotted per cell (n=60 cells per construct pooled from 3 independent experiments); Mann-Whitney test. All data are plotted as mean±SD; ****p<0.0001.

FIG. 10. Lipid nanoparticle delivery of the obscurin PH-domain suppresses PI3K/Akt activation and reduces migration. (A) Size (nm), polydispersity index (PDI), and zeta-potential (mV) values of formulations for Myr-Myc, Myr-oPH-Myc, Myr-kPH-Myc, and Myr-yPH-Myc lipid nanoparticle (LNP)/pDNA mixtures. (B-C) Immunoblots of MDA-MB-231 (B) and SKBr3 (C) cells probed for PI3K-p85 Y458 and pAkt T308/S473 phosphorylation levels treated with LNP-delivered Myr-Myc or Myr-oPH-Myc constructs. Densitometry values are presented as expression relative to Myr-Myc control, set at “1” (n=3 independent experiments); two-tailed t-test. (D-E) Representative brightfield images of transwell migration assays of MDA-MB-231 (D) and SKBr3 (E) cells treated with LNP-delivered Myr-Myc or Myr-oPH-Myc constructs. The percent (%) migrated cells per field are plotted, per treatment group (n=3-4 independent experiments); two-tailed t-test. All data are plotted as mean±SD; *p<0.05; **p<0.01.

FIG. 11. Lipid Nanoparticle Delivery of the obscurin PH-domain inhibits MMP expression and promotes apoptosis in breast cancer tumors. (A-B) qPCR of Myr-Myc (A) or Myr-oPH-Myc (B) pDNA constructs, plotted as pg pDNA per mg of digested tissue section from LNP-delivered Myr-Myc or Myr-oPH-Myc treated tumors (n=9 mice per group pooled from 2 independent experiments); Mann-Whitney test. (C) RT-qPCR for Myr-oPH-Myc mRNA expression, normalized to GAPDH, for Myr-Myc or Myr-oPH-Myc expressing tumors (n=9 mice per group pooled from 2 independent experiments); Mann-Whitney test. (D) Excised Myr-Myc and Myr-oPH-Myc LNP-treated primary tumors. (E) Immunoblots of Myr-Myc and Myr-oPH-Myc LNP-treated tumors for PARP and cleaved PARP. Densitometry values are presented as cleaved PARP expression relative to Myr-Myc control (n=9 mice per group pooled from 2 independent experiments); Mann-Whitney test. (F) qPCR of lymph nodes for human Long Interspersed Nuclear Element (hLINE) DNA from LNP-delivered Myr-Myc or Myr-oPH-Myc treated mice. hLINE DNA is plotted as μg hLINE DNA per mg of tissue section (n=9 mice per group pooled from 2 independent experiments); Mann-Whitney test. (G) Experimental mouse model schematic detailing LNP treatment of developing orthotopic MDA-MB-231 tumors. 1×10⁶ MDA-MB-231 cells were injected into the mammary fat pad of female NOD SCID mice on Day 0. Seven days following cell implantation, Myr-Myc or Myr-oPH-Myc LNPs were administered at the site of injection once a week for 6 weeks. Endpoint was specified as the point at which 6 total injections per construct had been administered to each mouse, followed by subsequent tissue harvest of the primary tumor, lungs, liver, and lymph nodes; schematic created with Biorender.com (agreement number WU289RLHPI). (H-I) qPCR of Myr-Myc (H) or Myr-oPH-Myc (I) pDNA constructs, plotted as pg pDNA per mg of digested tissue section from LNP-delivered Myr-Myc or Myr-oPH-Myc treated tumors (n=4-5 mice per group); two-tailed t-test (H) and Mann-Whitney test (I). (J) RT-qPCR for Myr-oPH-Myc mRNA expression, normalized to GAPDH, for Myr-Myc or Myr-oPH-Myc expressing tumors (n=4-5 mice per group); Mann-Whitney test. (K) Caliper measurements of tumor volumes throughout a 6-week course of intratumoral injection of either Myr-Myc or Myr-oPH-Myc LNPs (n=4-5 mice per group); two-way ANOVA. (L) qPCR of lymph nodes, lungs, and liver for hLINE DNA from Myr-Myc or Myr-oPH-Myc treated mice. hLINE DNA is plotted as pg hLINE DNA per mg tissue section; n=4-5 mice per group; Mann-Whitney test. (M) Immunoblots of Myr-Myc and Myr-oPH-Myc LNP-treated tumors for Pro-MMP9 and MMP9. Densitometry values are presented as Pro-MMP9 and MMP9 expression relative to the Myr-Myc control (n=4-5 mice per group); Mann-Whitney test. (N) cBioPortal Illumina HT-12 v3 microarray for MMP9 expression (TPM log₂) of invasive breast carcinoma patient samples from the 2024 TCGA and Genomic Data Commons dataset, separated by AAJC pathologic T stage (n=40-632 patient samples per T stage); Kruskal-Wallis test with Dunn's multiple comparison test. All data plotted as mean±SD (N) or mean±SEM (A-C, E, F, H-M); *p<0.05, ***p<0.001, ****p<0.0001.

FIG. 12. Myristoylated obscurin, kalirin, and PLCγ1 PH-domain sequesters PI3K-p85. (A) NCBI Blast analysis showing sequence homology between the obscurin PH-domain and the Kalirin or PLCγ1 PH-domains. (B) Schematic of plasmid DNA vectors encoding the myristoylated obscurin, kalirin, and PLCγ1 pleckstrin homology (PH)-domains, with C-terminal Myc-Tag; created with Biorender.com (LJ289RL920). (C) Representative confocal images of MDA-MB-231 cells transiently transfected with the Myr-Myc, Myr-oPH-Myc, Myr-kPH-Myc, or Myr-γ PH-Myc constructs; β-actin (magenta), DAPI (blue), Myc-tag (green), PI3K-p85 (orange), and merged Myc-tag/PI3K-p85 (yellow). Cells of each treatment group vary significantly in size; scale bars differ to visualize the entire cell. Corresponding line composite graphs display Myc-tag and PI3K-p85 gray values (pixel intensity).

FIG. 13. Myristoylated obscurin, and PLCγ1 PH-domains sequester PI3K-p85 to intracellular, membrane-bound vesicles. Representative images of STED super resolution microscopy of MDA-MB-231 cells transiently transfected with either the Myr-Myc, Myr-oPH-Myc, Myr-kPH-Myc, or Myr-γPH-Myc plasmid DNA constructs. Single MDA-MB-231 cells (same cells imaged in FIG. 6a) immunostained for Myc-tag (green) and PI3K-p85 (magenta) are shown at 60×, while zoom-in areas within the cell body marked in yellow are shown at 600× with areas of colocalization between Myc-tag and PI3K-p85 appearing white; PI3K-p85 cytoplasmic puncta that do not colocalize with Myc-Tag are denoted with yellow arrowheads.

FIG. 14. Sequestration of PI3K-p85 by myristoylated obscurin, kalirin, and PLCγ1 PH-domain modulates breast cancer cell morphology. (A-B) Cellular circularity (A) and cell area (μm²; B) of MDA-MB-231 cells transiently transfected with the obscurin, kalirin, or PLCγ1 PH-Myc or Myr-PH-Myc constructs (n=30 cells pooled from 3 independent experiments); two-way ANOVA with Tukey's (A) and Sidak's (B) multiple comparison test. (C) Representative confocal images of MDA-MB-231 cells transiently transfected with non-myristoylated oPH-Myc, kPH-Myc, or γPH-Myc constructs; β-actin (magenta), DAPI (blue), Myc-tag (green), PI3K-p85 (orange), and merged Myc-tag/PI3K-p85 (yellow). Cells of each treatment group vary significantly in size; scale bars differ to visualize the entire cell. Corresponding line composite graphs display Myc-tag and PI3K-p85 gray values (pixel intensity). All data are plotted as mean±SD; **p<0.01, ***p<0.001, ****p<0.0001.

FIG. 15. Alpha-fold structural modeling of the obscurin, kalirin, and PLCγ1 PH-domain/PI3K p85-SH3 complex predicts PH-domain binding to the SH3 dynamin binding interface. (A) Computational analysis schematic outlining the methodology used to predict the PH-domain binding site for p85-SH3. The obscurin, kalirin, or PLCγ1 PH-domain amino acid sequences were input into Alpha Fold along with the amino acid sequence of the PI3K-p85 SH3 domain. Select PH-domain residues at the PH/SH3 binding interface of each of the obscurin, kalirin, and PLCγ1 PH-domain/PI3K p85-SH3 complexes were “mutated” to ablate the interaction and subsequent molecular dynamics simulations were run to predict the critical PH-domain residues required for stable complex formation with p85-SH3; schematic created with Biorender.com (agreement number YC289RKFZU). (B) Equilibrated alpha-fold models of the obscurin (red), kalirin (blue), and PLCγ1 (green) PH-domains in complex with the dynamin binding interface (magenta) of the PI3K p85-SH3 domain; the remaining structure of the p85-SH3 domain appears in gray. (C-E) Alpha-Fold Predicted Local Distance Difference Test (pLDDT) confidence scoring of equilibrated PH-domain/p85-SH3 alpha-fold models for obscurin-PH (C), kalirin-PH (D) and PLCγ1-PH (E); the color scheme key indicates pLDDT scores >80 (orange and red) versus scores <80 (blue, green, and yellow). (F-H) Zoomed images of the Alpha-Fold predicted PH-domain:SH3-domain binding interface for obscurin (F), kalirin (G), and PLCγ1 (H); PH-domains (black) and critical residues (green); p85 SH3-domain (gray) and key residues (magenta); types of residue bonds are indicated underneath each panel as hydrophobic, electrostatic, or H-bonding. The shortest bond distance between residues is indicated with a dotted red line.

FIG. 16. Simulated mutagenesis and molecular dynamics analysis of the obscurin, kalirin, and PLCγ1 PH-domains predict the p85-SH3 Interacting Region (PSIR). (A-D) Simulated molecular dynamics of the obscurin PH-domain I23A, H21E/R107E, and I23A/H21E/R107E mutants. Radius of gyration (A°; A), Ca RMSD (root mean square deviation; B), RMSF (root mean square fluctuation) with indicated PH:SH3 interacting sites (green arrowheads; C), and average electrostatic interactions (red values above bars) between the obscurin PH-domain mutants and p85-SH3 domain (D) are plotted. (E-H) Simulated molecular dynamics of the kalirin PH-domain Q22A, E35A, K101A, and Q22A/E35A/K101A mutants. Radius of gyration (A°; E), Cα RMSD (F), RMSF with indicated PH:SH3 interacting sites (green arrowheads; G), and average electrostatic interactions (red values above bars) between the kalirin PH-domain mutants and p85-SH3 domain (H) are plotted. (I-L) Simulated molecular dynamics of the PLCγ1 PH-domain T100A, T8A, R23E, and T100A/T8A/R23E mutants. Radius of gyration (A°; I), C a RMSD (J), RMSF with indicated PH:SH3 interacting sites (green arrowheads; K), and average electrostatic interactions (red values above bars) between the PLCγ1 PH-domain mutants and P85-SH3 domain (L) are plotted. (M) Summary table of the obscurin, kalirin, and PLCγ1 PH-domain binding residues predicted to be critical for the PH-domain/p85-SH3 complex formation, as determined by simulated mutagenesis and molecular dynamics analysis. The PH-domain regions harboring the p85-SH3 binding residues are highlighted in red. The predicted local distance difference test (pLDDT) score, PH-domain interacting residues, p85-SH3 domain interacting residues, and interaction types are indicated. (N) Schematic of the PH-domain region including the second half of β-strand 1, first half of β-strand 2, and the β-strand 7/C-terminal α-helix linker (β-7 linker) that comprises the predicted p85-SH3 Interacting Region (PSIR; red); schematic created with Biorender.com (agreement number BI289RKODW).

The skilled artisan will understand that the drawings, described below, are for illustration purposes only. The drawings are not intended to limit the scope of the present teachings in any way.

### DETAILED DESCRIPTION

Current cancer therapies inhibit tumor growth but fail to target metastatic dissemination. Obscurin (720-870 kDa), a giant signaling protein that localizes to the breast epithelial cell membrane, is a metastasis suppressor that is commonly lost in breast cancer. Herein, the disclosure provides delivery of mini-obscurin—comprising the obscurin-pleckstrin homology (PH) domain, which is ~50-times smaller that the full-length protein—into aggressive breast cancer cells via adenovirus and lipid nanoparticles. Mechanistically, the obscurin-PH domain interacts with the PI3K-p85 regulatory subunit. Membrane-targeted obscurin-PH sequesters p85, suppressing PI3K/Akt activity. p85-sequestration eliminates filopodia, hampering migration and diminishing adhesion to pre-metastatic niche extracellular matrix substrates. This intervention further eradicates invadopodia and reduces matrix metalloproteinase expression, blocking invasion, dissemination, and metastasis. The phenotype is recapitulated using the structurally homologous kalirin and PLC γ 1 PH-domains, and this ultimately uncovers, via computational modeling, the p85-SH3-interacting-region, PSIR, that mediates p85-sequestration and PI3K inhibition. This disclosure provides a first-in-class group of non-chemical PI3K inhibitors, uniquely targeting the PI3K-p85 subunit, galvanizing the development of a novel gene therapy for the treatment of metastatic breast cancer.

Reference will now be made in detail to the presently preferred embodiments of the disclosure which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

The practice of the present invention employs, unless otherwise indicated, conventional techniques of molecular biology (including recombinant techniques), microbiology, cell biology, biochemistry and immunology, which are within the skill of the art. Such techniques are explained fully in the literature. See, e.g., Sambrook et al. Molecular Cloning: A Laboratory Manual, 2^{nd} edition (1989); Current Protocols in Molecular Biology (F. M. Ausubel et al. eds. (1987)); the series Methods in Enzymology (Academic Press, Inc.); PCR: A Practical Approach (M. MacPherson et al. IRL Press at Oxford University Press (1991)); PCR 2: A Practical Approach (M. J. MacPherson, B. D. Hames and G. R. Taylor eds. (1995)); Antibodies, A Laboratory Manual(Harlow and Lane eds. (1988)); Using Antibodies, A Laboratory Manual (Harlow and Lane eds. (1999)); and Animal Cell Culture (R. I. Freshney ed. (1987)).

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of”

As used herein, the term “about” means plus or minus 10% of the numerical value of the number with which it is being used.

The terms “nucleic acid,” and “polynucleotide,” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can also encompass analogues of natural nucleotides, as well as nucleotides that are modified in the base, sugar and/or phosphate moieties.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids. The term “sequence” relates to a nucleotide sequence of any length, which can be DNA or RNA; can be linear, circular or branched and can be either single-stranded or double stranded.

The term “identity” relates to an exact nucleotide-to-nucleotide or amino acid-to-amino acid correspondence of two polynucleotides or polypeptide sequences, respectively. Two or more sequences (polynucleotide or amino acid) can be compared by determining their percent identity. Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Sequence similarity” between polynucleotides can be determined by hybridization of polynucleotides under conditions that allow formation of stable duplexes between homologous regions, followed by digestion with single-stranded-specific nuclease(s), and size determination of the digested fragments.

“Subject,” as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In one embodiment, the subject is a human. In some embodiments, the subject is a mouse.

The term “therapeutically effective amount” or “effective amount” means the amount of the active component of the composition or method that is sufficient to show meaningful benefits, e.g, inhibition of cell migration or metastasis.

“Treatment” of an individual (e.g. a mammal, such as a human) or a cell is any type of intervention used in an attempt to alter the natural course of the individual or cell. Treatment includes, but is not limited to, administration of a therapeutic composition, and may be performed either prophylactically or subsequent to the initiation of a pathologic event or contact with an etiologic agent. Treatment includes any desirable effect on the symptoms or pathology of a disease or condition, e.g., inhibiting cancer metastasis, and may include, for example, minimal changes or improvements in one or more measurable effects of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

As used herein, “administering” refers to a method of delivering a composition to a subject or patient. A method of administration may be selected to target delivery (e.g., to specifically deliver) to a specific region or system of a body. For example, an administration may be parenteral (e.g., intramuscular).

The terms “subject” and “patient” are used interchangeably herein, and refer to an animal such as a mammal. In general, the terms refer to a human. The terms also includes domestic animals bred for food, sport, or as pets, including horses, cows, sheep, poultry, fish, pigs, cats, dogs, and zoo animals, goats, apes (e.g. gorilla or chimpanzee), and rodents such as rats and mice. Typical subjects include persons susceptible to, suffering from or that have suffered from cancer.

As used herein, the term “encapsulate” means to enclose, surround, or encase. A nucleic acid may be fully encapsulated, partially encapsulated, or substantially encapsulated. For example, in some embodiments, a nucleic acid of the disclosure may be encapsulated in a lipid nanoparticle.

“Lipid nanoparticles (LNPs)” refers to nanoscale lipid-based carriers capable of encapsulating therapeutic agents for targeted delivery.

As used herein, “nanoparticle” refers to a particle having any one structural feature on a scale of less than about 1000 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

### Nucleic Acids and Pharmaceutical Compositions

In one embodiment, the disclosure provides a nucleic acid encoding a polypeptide comprising a pleckstrin homology (PH) domain, a variant or fragment thereof, wherein the fragment of the PH domain comprises a p85-SH3 interacting region (PSIR). In some embodiments, the nucleic acid further encodes a myristoylation tag upstream of the pleckstrin homology (PH) domain, variant or fragment thereof.

In another embodiment, the disclosure provides a pharmaceutical composition comprising an effective amount of nucleic acid encoding a polypeptide comprising a pleckstrin homology (PH) domain, a variant or fragment thereof, wherein the fragment of the PH domain comprises a p85-SH3 interacting region (PSIR).

In some embodiments, the nucleic acid is RNA. In some embodiments, the nucleic acid is DNA. In some embodiments, the nucleic acid encodes a polypeptide that interacts with the PI3K-p85 regulatory subunit.

In some embodiments, the nucleic acid encoding the polypeptide encodes a myristoylation tag. In some embodiments, the myristoylation tag is located at the N-terminus of the polypeptide. In some embodiments, the myristoylation tag comprises an amino acid sequence of Met-Gly-Xaa-Xaa-Xaa-Ser/Thr-Xaa-Xaa. In some embodiments, the myristoylation tag comprises an amino acid sequence comprising Met-Gly-Ser-Ser-Lys-Ser-Lys-Pro-Lys (SEQ ID NO:7). In some embodiments, the myristoylation tag comprises a nucleotide sequence comprising ATGGGAAGCAGCAAGAGCAAGCCAAAG (SEQ ID NO:39). The nucleotide sequence can be RNA or DNA.

A pleckstrin homology (PH) domain is a protein domain found in many signaling proteins that helps target them to specific cellular membranes or partners by binding lipids (like phosphoinositides) or other proteins, playing crucial roles in cell signaling, intracellular trafficking, and cytoskeletal organization. These domains have a common beta-sandwich structure but vary in their specific lipid-binding abilities, often recognizing specific inositol phosphates (PIPs) to localize proteins to the plasma membrane or other compartments.

The source of the nucleic acid or polypeptide sequence that comprises the pleckstrin homology (PH) domain is not limiting. In some embodiments, the pleckstrin homology (PH) domain is from a human protein or nucleic acid sequence. In some embodiments, the source is artificial or synthetic.

In some embodiments, the source of the pleckstrin homology (PH) domain is obscurin, such as human obscurin. In some embodiments, human obscurin sequence is found in NCBI Accession No.: AJ002535. The amino acid and nucleic acid sequence encoding the PH domain of human obscurin is found in the table below. The nucleic acid sequence that encodes the PH domain can be either DNA or RNA.

TABLE 1
Human obscurin PH domain sequences.

| SEQ | |
|---|---|
| ID | |
| NO: | Sequence |
| 1 | Amino acid: |
| | LMENYPGTLQALGEPIRQGHFIVWEGAPGARMPWKGHNRHVFLF |
| | RNHLVICKPRRDSRTDTVSYVFRNMMKLSSIDLNDQVEGDDRAF |
| | EVWQEREDSVRKYLLQARTAIIKSSWVKEICGIQQRL |
| 2 | Nucleic acid: |
| | CTCATG GAGAACTACC CAGGCACCCT GCAGGCCCTG |
| | GGCGAGCCCA TCCGCCAGGG CCACTTCATC GTGTGGGAGG |
| | GTGCACCGGG GGCCCGCATG CCCTGGAAGG GCCACAACCG |
| | TCACGTGTTC CTCTTCCGCA ACCACCTGGT AATCTGCAAG |
| | CCCCGGCGAG ACTCCCGCAC CGATACCGTC AGCTACGTGT |
| | TCCGGAACAT GATGAAGCTG AGCAGCATCG ACCTGAACGA |
| | CCAGGTGGAG GGGGATGACC GCGCCTTCGA GGTGTGGCAG |
| | GAGCGGGAGG ACTCGGTGCG CAAGTACCTG CTGCAGGCAC |
| | GGACAGCCAT TATCAAGAGC TCGTGGGTGA |
| | AGGAGATCTG TGGCATCCAG CAGCGTCTG |

In some embodiments, the source of the pleckstrin homology (PH) domain is kalirin, such as human kalirin. In some embodiments, human kalirin sequence is found in NCBI Accession No.: KX533490. The amino acid and nucleic acid sequence encoding the PH domain of human kalirin is found in the table below. The nucleic acid sequence that encodes the PH domain can be either DNA or RNA.

TABLE 2
Human kalirin PH domain sequences.

| SEQ | |
|---|---|
| ID | |
| NO: | Sequence |
| 3 | Amino acid: |
| | MLEGFDENLDVQGELILQDAFQVWDPKSLIRKGRERHLFLFE |
| | ISLVFSKEIKDSSGHTKYVYKNKLLTSELGVTEHVEGDPCKF |
| | ALWSGRTPSSDNKTVLKASNIETKQEWIKNIREVIQERI |
| 4 | Nucleic acid: |
| | ATGC TGGAAGGGTT CGACGAGAAC CTGGATGTGC |
| | AGGGGGAGTT GATTCTCCAG GATGCCTTTC AAGTGTGGGA |
| | CCCGAAGTCG CTGATCCGGA AGGGGCGGGA GCGGCACTTG |
| | TTCCTCTTTG AGATCTCCTT GGTTTTTAGC AAGGAGATCA |
| | AAGATTCTTC AGGACACACG AAATATGTTT ACAAGAACAA |
| | GCTACTGACC TCAGAGCTGG GTGTGACCGA GCACGTGGAG |
| | GGCGATCCCT GCAAATTCGC CTTGTGGTCT GGGCGCACCC |
| | CATCCTCAGA CAATAAAACA GTGCTGAAAG CCTCCAACAT |
| | TGAAACCAAG CAGGAGTGGA TCAAGAACAT TCGAGAAGTG |
| | ATTCAAGAAA GGATC |

In some embodiments, the source of the pleckstrin homology (PH) domain is phospholipase-Cγ1 (PLCγ1), such as human PLCγ1. In some embodiments, human PLCγ1 sequence is found in NCBI Accession No.: NM_002660. The amino acid and nucleic acid sequence encoding the PH domain of human PLCγ1 is found in the table below. The nucleic acid sequence that encodes the PH domain can be either DNA or RNA.

TABLE 3
Human PLCγ1 PH domain sequences.

| SEQ | |
|---|---|
| ID | |
| NO: | Sequence |
| 5 | Amino acid: |
| | LEVGTVMTLFYSKKSQRPERKTFQVKLETRQITWSRGADKIEGAI |
| | DIREIKEIRPGKTSRDFDRYQEDPAFRPDQSHCFVILYGMEFRLK |
| | TLSLQATSEDEVNMWIKGLTWLMEDTLQAPTPL |
| 6 | Nucleic acid: |
| | CTCGAGG TGGGCACCGT CATGACTTTG TTCTACTCCA |
| | AGAAGTCGCA GCGACCCGAG CGGAAGACCT TCCAGGTCAA |
| | GCTGGAGACG CGCCAGATCA CGTGGAGCCG GGGCGCCGAC |
| | AAGATCGAGG GGGCCATTGA CATTCGTGAA ATTAAGGAGA |
| | TCCGCCCAGG GAAGACCTCA CGGGACTTTG ATCGCTATCA |
| | AGAGGACCCA GCTTTCCGGC CGGACCAGTC ACATTGCTTT |
| | GTCATTCTCT ATGGAATGGA ATTTCGCCTG AAAACGCTGA |
| | GCCTGCAAGC CACATCTGAG GATGAAGTGA ACATGTGGAT |
| | CAAGGGCTTA ACTTGGCTGA TGGAGGATAC ATTGCAGGCA |
| | CCCACACCCC TGC |

In some embodiments, the nucleic acid encodes a polypeptide comprising a pleckstrin homology (PH) domain of any one of SEQ ID NOS:1, 3 or 5, or a variant or fragment thereof, wherein the fragment of the PH domain comprises a p85-SH3 interacting region (PSIR).

In some embodiments, the nucleic acid encodes a polypeptide comprising a pleckstrin homology (PH) domain that is at least 90% identical to any one of SEQ ID NOS:1, 3 or 5. In some embodiments, the polypeptide comprises a pleckstrin homology (PH) domain that is at least 100% identical to any one of SEQ ID NOS: 1, 3 or 5.

In some embodiments, the polypeptide consists essentially of a pleckstrin homology (PH) domain, a variant or fragment thereof, wherein the fragment of the PH domain comprises a p85-SH3 interacting region (PSIR).

In some embodiments, the polypeptide consists essentially of a myristoylation tag and a pleckstrin homology (PH) domain, a variant or fragment thereof, wherein the fragment of the PH domain comprises a p85-SH3 interacting region (PSIR).

The nucleic acids used in the compositions and methods described herein include polynucleotide sequences that encode a pleckstrin homology (PH) domain, or fragments or variants thereof, such as polynucleotide sequences that encode a protein having at least 85% sequence identity (e.g., 85%, 90%, 95%, 96%, 97%, 98%, 99%, or more, sequence identity) to the amino acid sequence of SEQ ID NOS:1, 3 or 5, or a polynucleotide sequence encoding an amino acid sequence that contains one or more conservative amino acid substitutions relative to SEQ ID NOS:1, 3, or 5 (e.g., 1, 2, 3, 4, 5, 6, 7, 8, 9, 10, 11, 12, 13, 14, 15, 16, 17, 18, 19, 20 or more conservative amino acid substitutions). In some embodiments, the variant comprises an amino acid sequence that is at least 90% identical to SEQ ID NOS: 1, 3 or 5. In some embodiments, no more than 10% of the amino acids of the PH domain may be replaced with conservative amino acid substitutions.

Variants of the polynucleotide sequence can also be used, for example, due to degeneracy of codon usage.

The organismal source of the nucleic acid sequence encoding the PH domain or fragment or variant thereof is not limiting. In some embodiments, the PH domain can be a homolog of a human PH domain (e.g., obscurin, PLCγ1, kalirin) from another mammalian species (e.g., mouse, rat, cow, horse, goat, sheep, donkey, cat, dog, rabbit, guinea pig, or other mammal). In some embodiments, the nucleic acid sequence is derived from a mammal. In some embodiments, the nucleic acid sequence is of human origin.

The nucleic acid molecule(s) encoding the PH domain or fragments or variants thereof can be produced using recombinant DNA technology (e.g., polymerase chain reaction (PCR) amplification, cloning) or chemical synthesis. Nucleic acids that encode the PH domain or fragments or variants include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect (e.g., production of a PH domain or fragment or variant proteins in cells or other expression systems).

In some embodiments, the coding sequence of the PH domain is encoded by SEQ ID NOS:2, 4, or 6. The nucleic acid encoding a PH domain in accordance with the invention may contain a variety of different bases compared to the wild-type sequence and yet still encode a corresponding polypeptide that exhibits the biological activity of the PH domain polypeptide.

In some embodiments, a particular nucleotide sequence encoding a PH domain polypeptide may be identical over its entire length to the coding sequence in SEQ ID NOS:2, 4 or 6. In some embodiments, a particular nucleotide sequence encoding a PH domain polypeptide may be an alternate form of SEQ ID NOS:2, 4 or 6 due to degeneracy in the genetic code or variation in codon usage encoding the polypeptides of SEQ ID NOS:1, 3 or 5.

In some embodiments, the nucleic acid sequence of a PH domain can contain a nucleotide sequence that is highly identical, at least 60% identical, with a nucleotide sequence encoding a PH domain polypeptide. In some embodiments, the nucleic acid sequence of a PH domain comprises a nucleotide sequence that is at least 60%, 65%, 70%, 75%, 80%, 85%, 90%, 91%, 92%, 93%, 94%, 95%, 96%, 97%, 98% 99% or 100% identical with the encoding nucleotide sequence set forth in SEQ ID NOS:2, 4, or 6.

When a polynucleotide of the invention is used for the production of a PH domain polypeptide or fragment or variant thereof, the polynucleotide may include the coding sequence for the polypeptide or fragment or variant thereof, by itself, the coding sequence for the polypeptide or fragment or variant thereof in reading frame with other coding sequences, such as those encoding a myristoylation tag sequence, or other fusion peptide portions. The polynucleotide may also contain non-coding 5′ and 3′ sequences, such as transcribed, non-translated sequences, splicing and polyadenylation signals, ribosome binding sites and sequences that stabilize mRNA.

In some embodiments, the nucleotide sequence encoding the PH domain or fragment or variant thereof includes nucleic acid molecules comprising a polynucleotide having a nucleotide sequence at least 90%, 91%, 92%, 93%, 94%, 95%, 96%, 97%, 98% 99% or 100% identical to (a) a nucleotide sequence encoding a PH domain having the amino acid sequence in SEQ ID NOS:1, 3, or 5; or (b) a nucleotide sequence complementary to the nucleotide sequences in (a).

Conventional means utilizing known computer programs such as the BestFit program (Wisconsin Sequence Analysis Package, Version 10 for Unix, Genetics Computer Group, University Research Park, 575 Science Drive, Madison, Wis. 53711) may be utilized to determine if a particular nucleic acid or polypeptide molecule is at least 90%, 91%, 92%, 93%, 94%, 95%, 96%, 97%, 98% or 99% identical to SEQ ID NOS:1-6, for example.

In some embodiments, the nucleotide sequence used encoding a PH domain or fragments or variants thereof encodes an amino acid sequence of SEQ ID NOS: 1, 3 or 5, in which 1, 2, 3, 4, 5, 6, 7, 8, 9, 10 or more amino acid residues are substituted, deleted or added, in any combination.

In some embodiments, the nucleic acid molecule encodes a fragment of a PH domain which is capable of suppressing PI3k/Akt activity. In some embodiments, the biologically active fragment can be at least about 80, 85, 90, 95, 100, 105, 110, 111, 112, 113, 114. 115, 116, 117, 118, 119, 120, 121, 122, 123 or 124 amino acids in length.

In some embodiments, stable expression of a PH domain or fragments or variants thereof in a cancer can be achieved by integration of the polynucleotides containing them into the nuclear genome of the cell. A variety of vectors for the delivery and integration of polynucleotides encoding exogenous protein into the nuclear DNA of a mammalian cell have been developed. In some embodiments, expression vectors for use in the compositions and methods described herein contain a polynucleotide sequence that encodes a PH domain or fragments or variants thereof, as well as, e.g., additional sequence elements used for the expression of these agents and/or the integration of these polynucleotide sequences into the genome of a mammalian cell. Certain vectors that can be used for the expression of a PH domain or fragments or variants thereof include plasmids that contain regulatory sequences, such as promoter and enhancer regions, which direct gene transcription. Other useful vectors for expression of a PH domain or fragments or variants thereof contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

In some embodiments, the compositions and methods described herein increase the expression of a PH domain or fragments or variants thereof by administering a nucleic acid vector(s) or composition that contains a polynucleotide encoding a PH domain or fragments or variants thereof.

Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

In some embodiments, a PH domain or fragments or variants thereof can also be introduced into a mammalian cell by targeting vectors encoding a PH domain or fragments or variants thereof to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a PH domain or fragments or variants thereof by mammalian RNA polymerase is important for gene expression. As such, one may include sequence elements within the polynucleotide that exhibit a high affinity for transcription factors that recruit RNA polymerase and promote the assembly of the transcription complex at the transcription initiation site.

Such sequence elements include, e.g., a mammalian promoter, the sequence of which can be recognized and bound by specific transcription initiation factors and ultimately RNA polymerase.

Polynucleotides suitable for use in the compositions and methods described herein also include those that encode a PH domain or a fragment or variant thereof protein downstream of a mammalian promoter. Promoters that are useful for the expression in mammalian cells include ubiquitous promoters. Ubiquitous promoters include the CAG promoter, or the cytomegalovirus (CMV) promoter. Cell type and tissue specific promoters can also be utilized.

Alternatively, promoters derived from viral genomes can also be used for the stable expression of these agents in mammalian cells. Examples of functional viral promoters that can be used to promote mammalian expression of these agents include adenovirus late promoter, vaccinia virus 7.5K promoter, SV40 promoter, tk promoter of HSV, mouse mammary tumor virus (MMTV) promoter, LTR promoter of HIV, promoter of Moloney virus, Epstein barr virus (EBV) promoter, and the Rous sarcoma virus (RSV) promoter.

In some embodiments, the nucleic acid(s) encoding a PH domain or fragment or variant are delivered to cells by a viral vector. In some embodiments, the virus is a DNA virus (e.g., dsDNA or ssDNA virus). In other embodiments, the virus is an RNA virus (e.g., an ssRNA virus). Exemplary viral vectors/viruses include retroviruses, lentiviruses, adenovirus, adeno-associated virus (AAV), vaccinia viruses, poxviruses, and herpes simplex viruses.

In some embodiments, the virus infects dividing cells. In other embodiments, the virus infects non-dividing cells. In some embodiments, the virus infects both dividing and non-dividing cells. In some embodiments, the virus can integrate into the host genome. In some embodiments, the virus is engineered to have reduced immunity, e.g., in humans. In some embodiments, the virus is replication-competent. In other embodiments, the virus is replication-defective, e.g., having one or more coding regions for the genes necessary for additional rounds of virion replication and/or packaging replaced with other genes or deleted. In some embodiments, the virus causes transient expression of the PH domain or fragment or variant protein. In other embodiments, the virus causes long-lasting, e.g., at least 1 week, 2 weeks, 1 month, 2 months, 3 months, 6 months, 9 months, 1 year, 2 years, or permanent expression, of the PH domain or fragment or variant protein. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the nucleic acid is delivered by a recombinant retrovirus. In some embodiments, the retrovirus (e.g., Moloney murine leukemia virus) comprises a reverse transcriptase, e.g., that allows integration into the host genome. In some embodiments, the retrovirus is replication-competent. In other embodiments, the retrovirus is replication-defective, e.g., having one of more coding regions for the genes necessary for additional rounds of virion replication and packaging replaced with other genes, or deleted.

In some embodiments, the nucleic acid is delivered by a recombinant lentivirus. In some embodiments, the lentivirus is replication-defective and does not comprise one or more genes required for viral replication.

In some embodiments, the nucleic acid is delivered by a recombinant adenovirus. In some embodiments, the adenovirus is engineered to have reduced immunity in human. In some embodiments, the nucleic acid is delivered by a recombinant AAV. In some embodiments, the AAV can incorporate its genome into that of a host cell. In some embodiments, the AAV is a self-complementary adeno-associated virus (scAAV), e.g., a scAAV that packages both strands which anneal together to form double stranded DNA. AAV serotypes that can be used in the methods of the invention include, e.g., AAV1, AAV2, modified AAV2 (e.g., modifications at Y444F, Y500F, Y730F and/or S662V), AAV3, modified AAV3 (e.g., modifications at Y705F, Y731 F and/or T492V), AAV4, AAV5, AAV6, modified AAV6 (e.g., modifications at S663V and/or T492V), AAV8, AAV 8.2, AAV9, AAV rh 10, and pseudotyped AAV, such as AAV2/8, AAV2/5 and AAV2/6 can also be used in the disclosed methods.

In some embodiments, the nucleic acid is delivered by a hybrid virus, e.g., a hybrid of one or more of the viruses described herein.

In some embodiments, a packaging cell can be used to form a virus particle that is capable of infecting a host or target cell. Such a cell can include a 293 cell, which can package adenovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed. For example, an AAV vector typically only possesses inverted terminal repeat (ITR) sequences from the AAV genome which are required for packaging and gene expression in the host or target cell. The missing viral functions can be supplied in trans by the packaging cell line. The viral nucleic acid can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the nucleic acid is delivered by a non-vector based method (e.g., using naked DNA or DNA complexes). For example, the nucleic acid can be delivered by organically modified silica or silicate (Ormosil), electroporation, gene gun, sonoporation, magnetofection, lipid-mediated transfection, dendrimers, inorganic nanoparticles, calcium phosphates, or a combination thereof.

In some embodiments, the nucleic acid is delivered by a combination of a vector and a non-vector based method. For example, a virosome comprises a liposome combined with an inactivated virus (e.g., HIV or influenza virus), which can result in more efficient gene transfer than either a viral or a liposomal method alone.

The PH domain or fragments or variants thereof constructs described herein may be delivered or introduced into a cancer cell by any suitable means, including, for example, by injection of mRNA or accordingly nucleic acid, for example, a CDNA, CRNA, or IRNA. See, Hamrnerschmidt et al. (1999) Methods Cell Biol. 59:87-115. In some embodiments, nucleic acid can be delivered into cells by microinjection, electroporation, lipid-mediated transfection, peptide-mediated delivery, or a combination thereof.

In some embodiments, the pharmaceutical composition comprises lipid nanoparticles (LNPs) that encapsulate the nucleic acid for delivery to cells. In some embodiments, the LNPs comprise a mixture of multiple components and excipients including ionizable lipid, helper lipid, PEG-lipid, and cholesterol or derivatives thereof. In some embodiments, the nucleic acid that is encapsulated is RNA or DNA.

In some embodiments, the LNP comprises an ionizable lipid. In some embodiments, the ionizable lipid is selected from Dlin-KC2-DMA (2,2-dilinoleyl-4-dimethylaminoethyl-[1,3]-dioxolane), DLin-MC3-DMA, SM-102, ALC-0315, 306Oi10, FTT5, C12-200, BAMEA-O16B and combinations thereof.

In some embodiments, the LNP comprises a helper lipid. In some embodiments, the nanoparticle comprises a helper lipid selected from the group consisting of DSPC (distearoylphosphatidylcholine), DPPC (dipalmitoylphosphatidylcholine), HSPC (hydrogenated soy phosphatidylcholine), DOPC (1,2-dioleoyl-sn-glycero-3-phosphocholine), DOPE (1,2-dioleoyl-sn-glycero-3-phosphoethanolamine), and DOPG (1,2-dioleoyl-sn-glycero-3-phosphoglycerol), and combinations thereof. In some embodiments, the nanoparticle comprises DSPC (distearoylphosphatidylcholine).

In some embodiments, the LNP comprises a pegylated lipid. In some embodiments, the nanoparticle comprises a combination of pegylated lipids. In some embodiments, the nanoparticle comprises a pegylated lipid selected from the group consisting of DSPE-PEG2000 (1,2-distearoyl-sn-glycero-3-phosphoethanolamine-N-[methoxy(polyethylene glycol)-2000]), DMG-PEG2000 (1,2-dimyristoyl-sn-glycero-3-methoxypolyethylene glycol-2000), DOPE-PEG (1,2-dioleoyl-sn-glycero-3-phosphoethanolamine-N-[methoxy(polyethylene glycol)]), ALC-0159, ALC-0315 and combinations thereof.

In some embodiments, the LNP comprises cholesterol or a derivative thereof. In some embodiments derivatives include β-sitosterol, stigmasterol, and synthetic analogs such as DC-Cholesterol (cationic) and modified cholesterol (e.g., BHEM-Cholesterol).

In some embodiments, the molar ratio of ionizable lipid:helper lipid:cholesterol:PEG lipid ranges from 30-50:5-15:25-40:0.5-5. In some embodiments, the molar ratio of ionizable lipid:helper lipid:cholesterol:PEG lipid is about 50:10.5:38.1:1.5.

In some embodiments, the lipid nanoparticles comprise Dlin-KC2-DMA (2,2-dilinoleyl-4-dimethylaminoethyl-[1,3]-dioxolane), DSPC (1,2-distearoyl-sn-glycero-3-phosphocholine), cholesterol, and mPEG2000-DSPE (1,2-dimyristoyl-rac-glycero-3-methoxypolyethyleneglycol-2000.

In some embodiments, the molar ratio of KC2:DSPC:cholesterol:mPEG2000-DSPE ranges from 30-50:5-15:25-40:0.5-5. In some embodiments, the molar ratio of KC2:DSPC:cholesterol:mPEG2000-DSPE is about 50:10.5:38.1:1.5.

In some embodiments, the lipid nanoparticles have a nitrogen-to-phosphate ratio (N/P) of about 5-10. In some embodiments, the lipid nanoparticles have a nitrogen-to-phosphate ratio (N/P) of about 8.

In some embodiments, pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

Formulations of the pharmaceutical compositions described herein can be prepared by any method known or hereafter developed in the art of pharmacology. In general, such preparatory methods include the step of bringing the nanoparticles into association with an excipient and/or one or more other accessory ingredients, and then, if necessary and/or desirable, dividing, shaping and/or packaging the product into a desired single- or multi-dose unit.

Relative amounts of the nanoparticles, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) lipid nanoparticles.

The nucleic acids can be formulated using one or more excipients. The complexes described herein can be formulated using one or more excipients to: (1) increase stability; (2) increase cell transfection; (3) permit the sustained or delayed release (e.g., from a depot formulation of the complex); (4) alter the biodistribution (e.g., target the polynucleotide to specific tissues or cell types); or (5) alter the release profile therapeutic agent in vivo.

In some embodiments, the pharmaceutical formulation comprises a delivery agent comprising an ionizable amino lipid, a helper lipid (e.g., DSPC), a sterol (e.g., Cholesterol), and a PEG lipid (e.g., PEG-DMG), e.g., with a mole ratio in the range of about (i) 40-50 mol % ionizable amino lipid, optionally 45-50 mol % ionizable amino lipid, for example, 45-46 mol %, 46-47 mol %, 47-48 mol %, 48-49 mol %, or 49-50 mol %, for example about 45 mol %, 45.5 mol %, 46 mol %, 46.5 mol %, 47 mol %, 47.5 mol %, 48 mol %, 48.5 mol %, 49 mol %, or 49.5 mol %; (ii) 30-45 mol % sterol (e.g., cholesterol), optionally 35-42 mol % sterol, for example, 30-31 mol %, 31-32 mol %, 32-33 mol %, 33-34 mol %, 35-35 mol %, 35-36 mol %, 36-37 mol %, 37-38 mol %, 38-39 mol %, or 39-40 mol %, or 40-42 mol % sterol; (iii) 5-15 mol % helper lipid (e.g., DSPC), optionally 10-15 mol % helper lipid, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % helper lipid; and (iv) 1-5% PEG lipid, optionally 1-5 mol % PEG lipid, for example 1.5 to 2.5 mol %, 1-2 mol %, 2-3 mol %, 3-4 mol %, or 4-5 mol % PEG lipid.

A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, MD, 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC®F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Oxidation can be a potential degradation pathway for nucleic acids. In order to prevent oxidation, in some embodiments, antioxidants can be added to the pharmaceutical composition. Exemplary antioxidants include, but are not limited to, alpha tocopherol, ascorbic acid, ascorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of pharmaceutical composition is maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

In some embodiments, the pharmaceutical composition described herein can contain a cryoprotectant to stabilize a nucleic acid described herein during freezing. Exemplary cryoprotectants include, but are not limited to mannitol, sucrose, trehalose, lactose, glycerol, dextrose, etc., and combinations thereof.

In some embodiments, the pharmaceutical composition can contain a bulking agent in lyophilized formulations to yield a “pharmaceutically elegant” cake, stabilize the lyophilized nanoparticles during long-term (e.g., 36 month) storage. Exemplary bulking agents of the present disclosure can include, but are not limited to sucrose, trehalose, mannitol, glycine, lactose, raffinose, and combinations thereof.

In some embodiments, the pharmaceutical composition can be in the form of or comprise, without limitation, viral vectors, liposomes, lipid nanoparticles, lipidoids, polymers, lipoplexes, microvesicles, exosomes, nanoparticle mimics, nanotubes, and combinations thereof.

The pharmaceutical compositions described herein can be prepared by any method known or hereafter developed in the art of pharmacology.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one specific nucleic acid. As a non-limiting example, the compositions can contain 1, 2, 3, 4, or 5 different nucleic acid sequences.

The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

### Therapeutic Methods

In another embodiment, the disclosure provides a method of treating cancer in a subject, comprising administering to the subject an effective amount of a nucleic acid or pharmaceutical composition herein.

As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the cancer is non-small-cell lung cancer, nonpapillary renal cell carcinoma, cervical carcinoma, ovarian carcinoma (e.g., serous ovarian carcinoma), or breast carcinoma. The cancers may be characterized in various other ways including, but not limited to, serous, endometrioid, mucinous, clear cell, Brenner, or undifferentiated.

The cancer to be treated is not limiting. In some embodiments, the cancer is breast cancer. In some embodiments, the breast cancer is metastatic breast cancer. In some embodiments, the cancer is a HER2-positive. In some embodiments, the cancer is a HER2 over-expressing or HER2 high-expressing cancer. In some embodiments, the cancer is a HER2 low-expressing cancer. In some embodiments, the cancer is a Her2-negative tumor or cancer. In some embodiments, the cancer is triple-negative breast cancer (TNBC).

In some embodiments, the administering suppresses PI3K/Akt activity in cancer cells as evidenced by reduced phosphorylation levels of activating p85-Tyr458 as well as Akt-Thr308 and -Ser473.

In some embodiments, the administering inhibits formation of filopodia in cancer cells.

In some embodiments, the administering inhibits migration and adhesion of cancer cells to pre-metastatic niche extracellular matrix substrates.

In some embodiments, the administering inhibits invadopodia and matrix metalloproteinase expression.

The composition can be administered to a subject by a variety of methods available to the art, including but not limited to localized injection, catheter administration, systemic injection, intraperitoneal injection, parenteral administration, intra-arterial injection, intravenous injection, transvascular injection, intramuscular injection, subcutaneous placement/injection, surgical injection into a tissue of interest (e.g., injection into a tumor) or via direct application to tissue surfaces (e.g., during surgery).

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Delivery of Pleckstrin Homology Domains Suppresses PI3K/Akt Signaling and Breast Cancer Metastasis

In this example, we describe the development of mini-obscurin—comprising the obscurin-pleckstrin homology (PH) domain, which is ~50-times smaller that the full-length protein—and delivery of the mini-obscurin into aggressive breast cancer cells via adenovirus and lipid nanoparticles.

### The Obscurin-PH Domain Suppresses PI3K/Akt Signaling and Breast Cancer Cell Migration

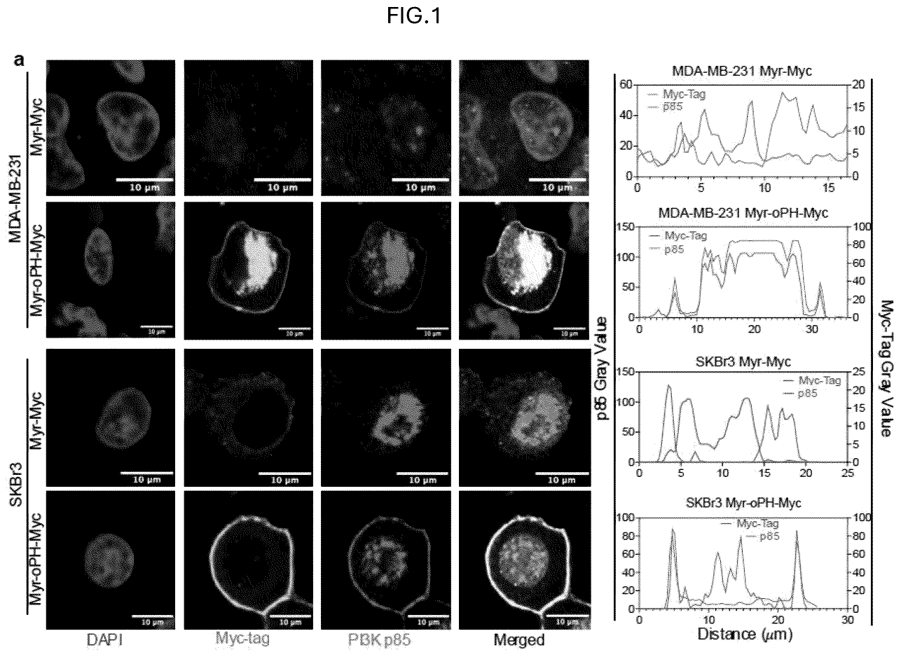

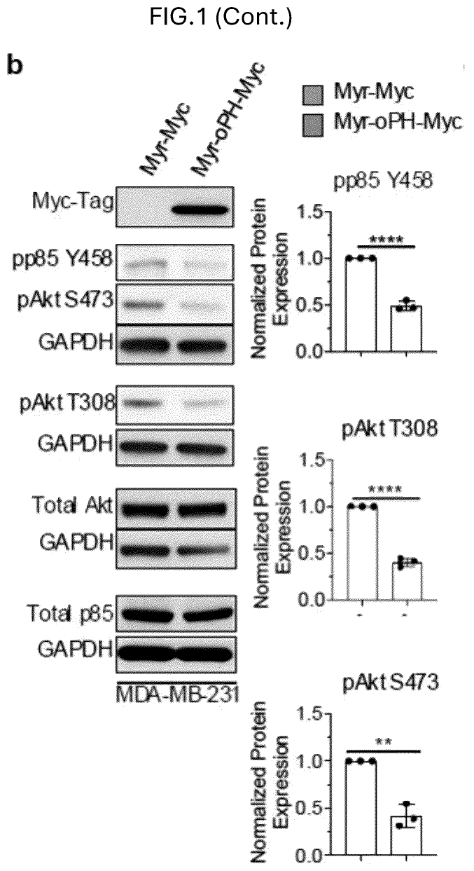

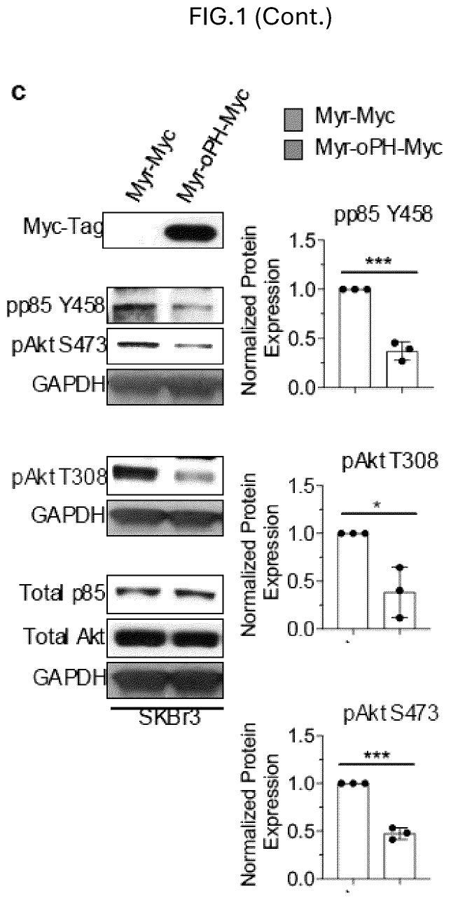

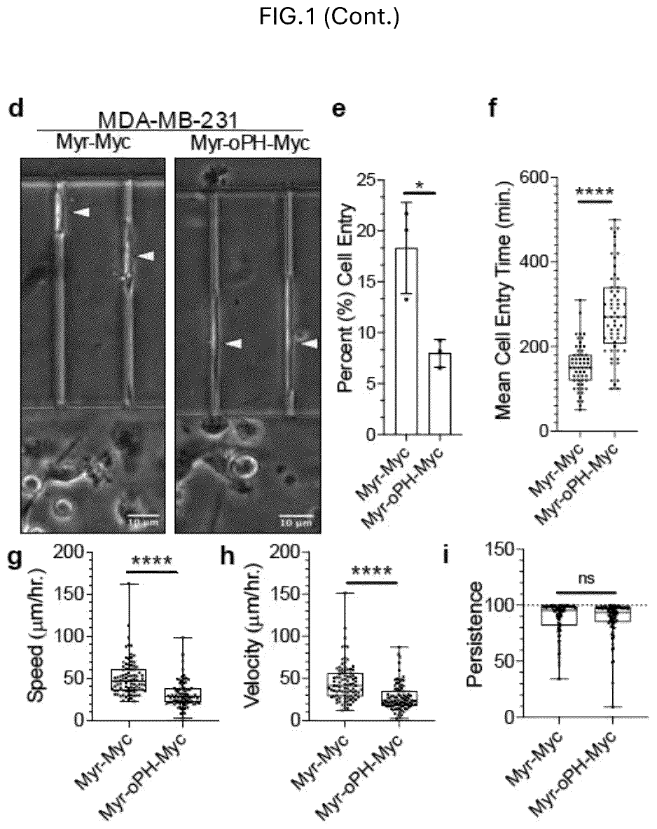

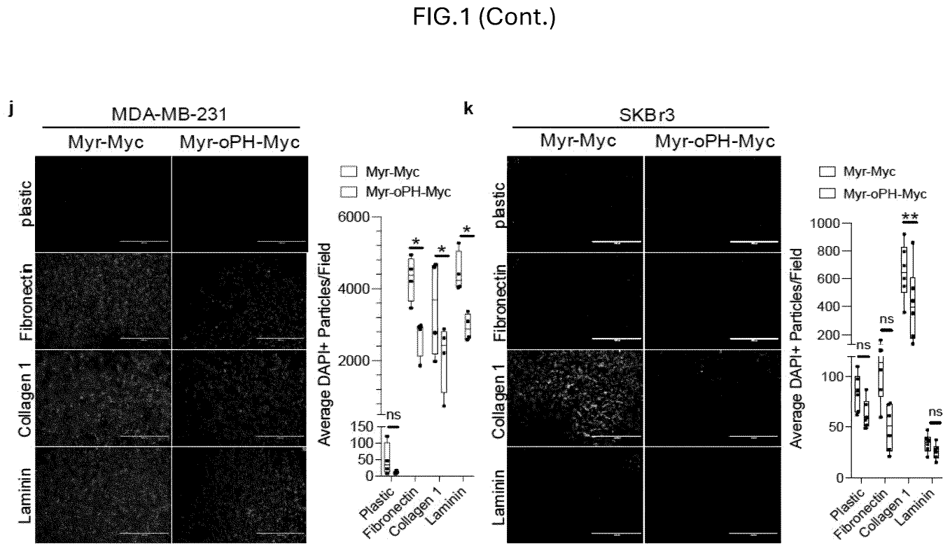

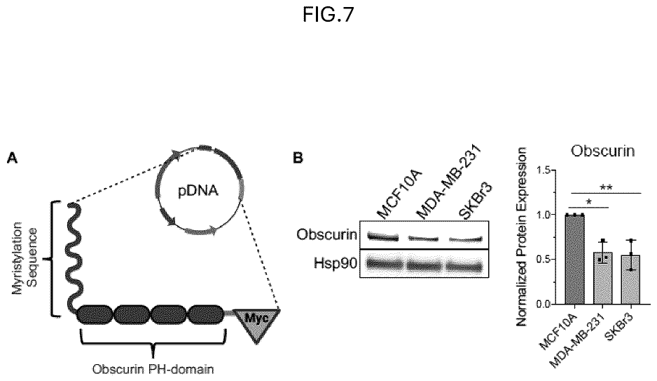

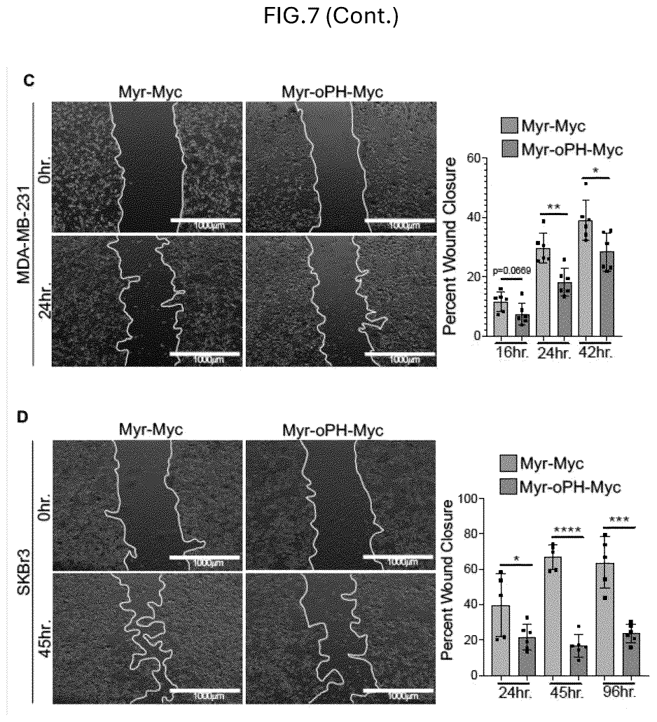

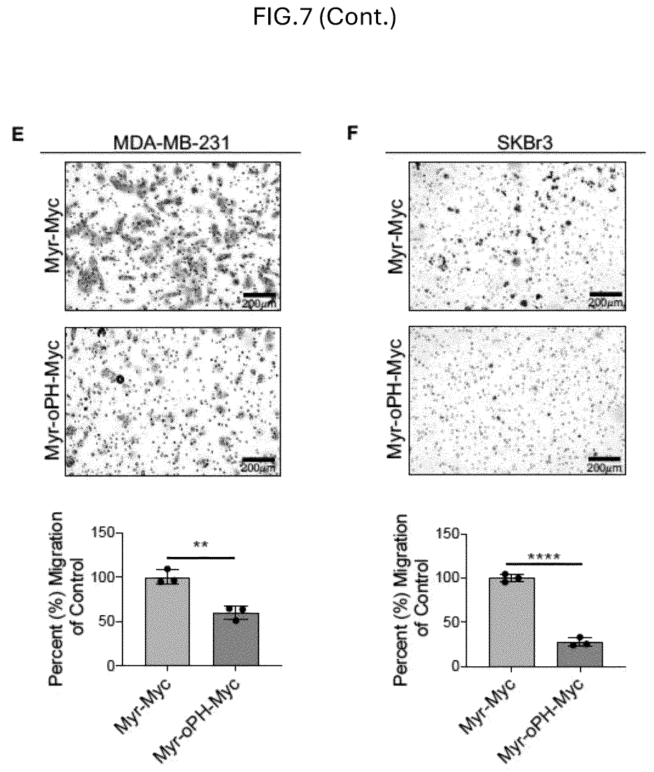

The obscurin-PH domain binds directly to the PI3K-p85 regulatory subunit, and small molecule inhibitors targeting the PI3K/Akt axis in obscurin-deficient breast epithelial cells block cell migration and invasion (M. Shriver et al., Oncotarget, (2016), 7:45414). We therefore set forth to explore the suppressive potential of the obscurin-PH domain on PI3K/Akt activity and downstream cellular processes that mediate metastatic spread. To mobilize the obscurin PH-domain to the cell membrane, we added a myristoylation tag at its N-terminus along with a Myc-tag at its C-terminus for ease of detection (FIG. 7A) (J. M. Rhee et al., Genesis, (2006), 44:202). We then asked if ectopic expression of myristoylated obscurin-PH domain in highly aggressive triple negative/claudin-low MDA-MB-231 and HER2+ SKBr3 breast cancer cell lines, which exhibit ~50% reduction in obscurin levels compared to non-tumorigenic MCF10A breast epithelial cells (FIG. 7B), restores the anti-metastatic function of full-length obscurin. Adenovirally-mediated expression of myristoylated obscurin-PH (Myr-oPH-Myc) in either cell line confirmed its cell membrane/perinuclear localization (FIG. 1A) and robust expression (FIG. 1B, C). Although detection of control Myr-Myc protein (<1 kDa) was not feasible via immunoblotting, a diffuse faint distribution was observed via immunofluorescence (FIG. 1A). Ectopic expression of Myr-oPH-Myc, but not control Myr-Myc protein, led to re-distribution of the PI3K-p85 subunit from a nuclear/cytoplasmic punctate localization to a cell membrane/perinuclear accumulation, coincident with Myr-oPH-Myc (FIG. 1A). The Myr-oPH-Myc mediated translocation of the PI3K-p85 subunit was accompanied by significant suppression of PI3K/Akt activity, as evidenced by the reduced phosphorylation levels of activating p85-Tyr458 as well as Akt-Thr308 and -Ser473 (FIG. 1B, C), whereas total p85 and Akt levels remained unchanged (FIG. 1B, C).

Consistent with the critical role of the PI3K/Akt axis in cancer cell migration, ectopic expression of Myr-oPH-Myc in MDA-MB-231 and SKBr3 cells markedly reduced collective (FIG. 7C, D), and chemotactic (FIG. 7E, F) cell migration, as assessed by wound healing and Transwell migration assays, respectively (M. Shriver et al., Oncotarget, (2016), 7:45414; C. Jimenez et al., J Cell Biol, (2000), 151:249; S. Corallino et al., Nat Commun, (2018), 9:1475; R. Zhou et al., Horm Metab Res, (2014), 46:753). Single cell migration was similarly affected, as assessed in confining microchannels of 10 μm height×3 μm width (FIG. 1D), where MDA-MB-231 cells transduced with Myr-oPH-Myc exhibited significantly decreased capability to enter the microchannels (FIG. 1E) and required significantly longer time to do so (FIG. 1F), compared to cells expressing control Myr-Myc. Moreover, Myr-oPH-Myc transduced MDA-MB-231 cells displayed considerably reduced speed (FIG. 1G) and velocity (FIG. 1H), although persistence (net displacement/total distance traveled) was unaltered (FIG. 1I).

### The Obscurin PH-Domain Decreases Breast Cancer Cell Adhesion and Ablates Filopodia Formation

Cancer cells shedding from primary breast tumors metastasize and adhere to secondary organ sites by utilizing pre-metastatic niche-specific extracellular matrix (ECM) proteins (Y. Li et al., Theranostics, (2023), 13:230). Prior work has shown that collagen I mediates colonization of the bone and peritoneal pre-metastatic niches, fibronectin the pulmonary and liver pre-metastatic niches, and laminin the brain pre-metastatic niche (H. Peinado et al., Nat Rev Cancer, (2017, 17 (5), 302). To interrogate if restoration of obscurin-PH expression impedes adhesion to pre-metastatic niche ECM proteins, we performed adhesion assays on plastic, collagen I, fibronectin, and laminin. MDA-MB-231 cells expressing control Myr-Myc protein ably adhered on all three pre-metastatic ECM substrates (FIG. 1J), consistent with the highly aggressive nature of their triple negative/claudin-low molecular subtype that promiscuously metastasizes to multiple organ sites (A. D. H. Wenjing Chen et al., J Clin Oncol, (2010), 28:3271). Contrary to MDA-MB-231, SKBr3 cells transduced with control Myr-Myc protein adhered effectively to collagen I, but not fibronectin or laminin, in agreement with their HER2+ molecular phenotype that robustly colonizes the peritoneum (H. Kennecke et al., J Clin Oncol, (2010), 28:3271). (FIG. 1K). Interestingly, MDA-MB-231 cells expressing Myr-oPH-Myc exhibited indiscriminately reduced adhesion on all three pre-metastatic niche ECM substrates (FIG. 1J), and SKBr3 cells expressing Myr-oPH-Myc displayed decreased adhesion to collagen I (FIG. 1K).

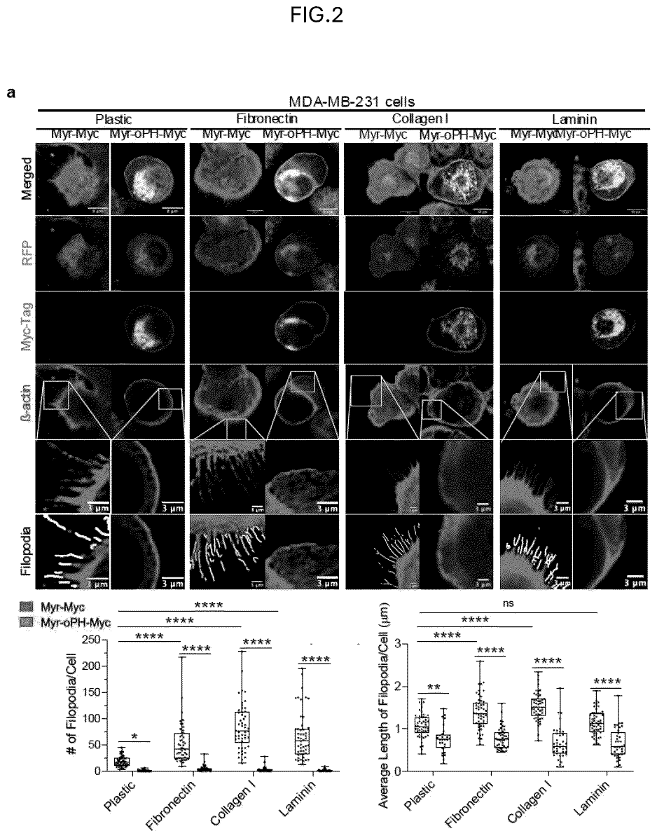

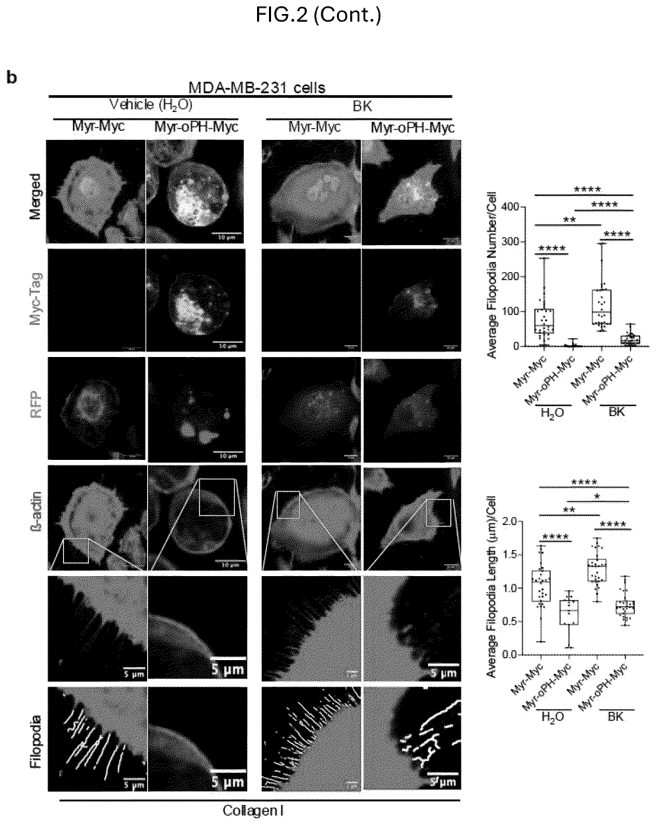

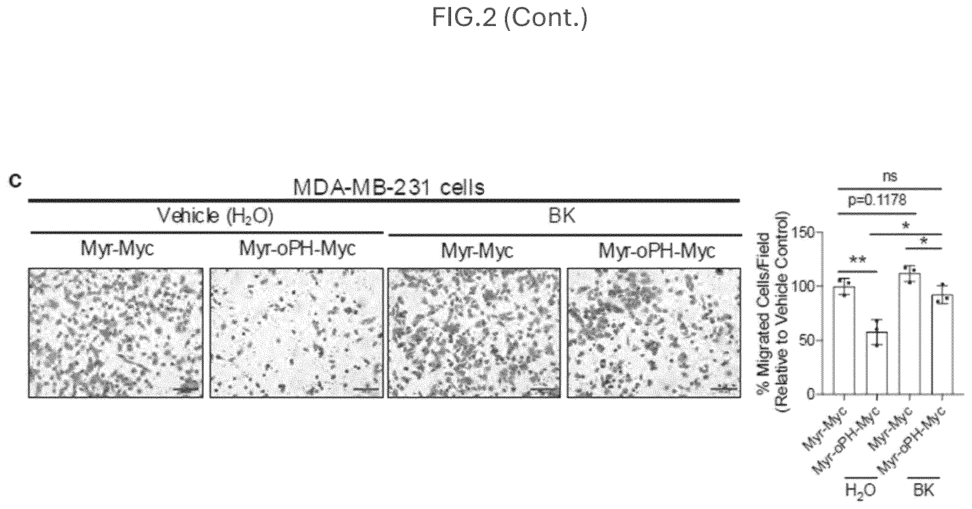

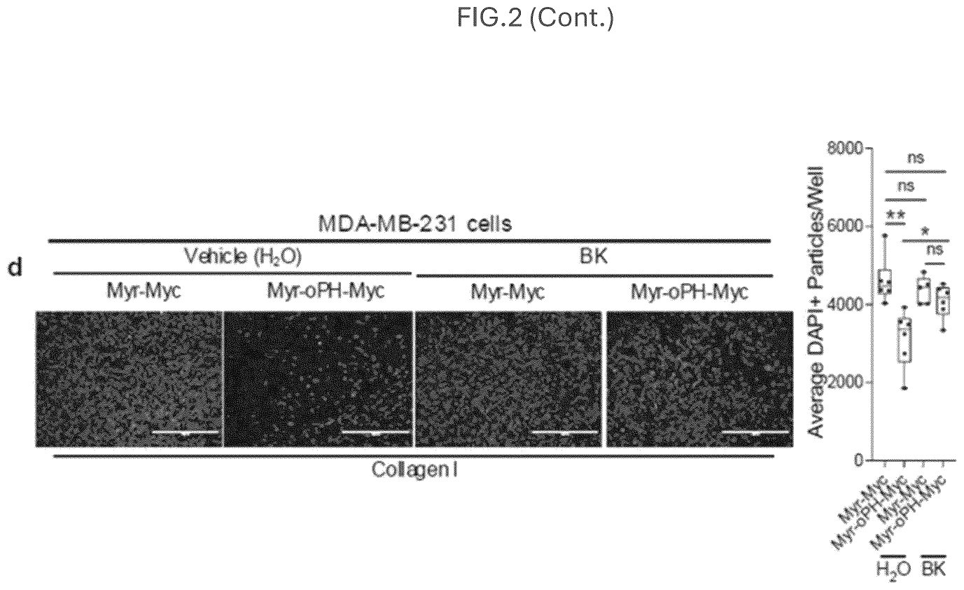

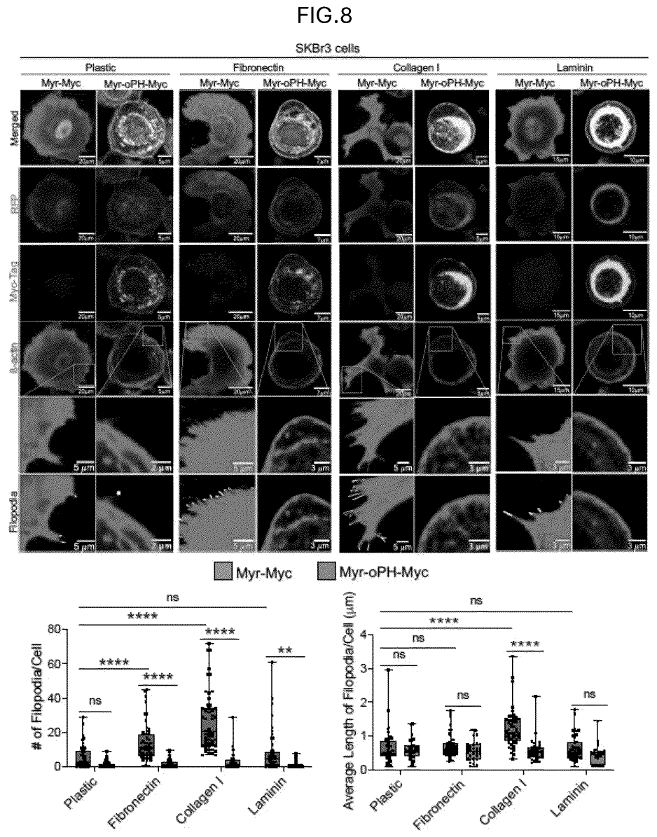

Actin is a pinnacle driver of cell morphological changes, and activated PI3K/Akt signaling orchestrates cell migration, adhesion, and metastasis through shifts in actin nucleation (M. Izdebska et al., Biomed Res Int, (2018), 2018:4578373). This actin remodeling gives rise to filopodia, which are dynamic, actin-rich structures of >1 μm in length protruding from the cell edge (R. Kozma et al., Mol Cell Biol, (1995), 15:1942). We therefore assessed filopodia formation via actin immunostaining of Myr-Myc and Myr-oPH-Myc expressing MDA-MB-231 and SKBr3 cells. Successfully transduced cells were identified via RFP fluorescence (expressed by the bicistronic adenoviral vector) in addition to Myc-tag immunolabeling. True to their highly aggressive phenotype, MDA-MB-231 cells expressing control Myr-Myc extended filopodia on plastic (FIG. 2A), a property that is markedly enhanced in the presence of the ECM proteins fibronectin, collagen I, and laminin (FIG. 2A), in agreement with earlier reports (A. Lepucki et al., J Clin Med, (2022), 11 (5). Remarkably, Myr-oPH-Myc expressing MDA-MB-231 cells failed to form filopodia, irrespective of the surrounding ECM environment (FIG. 2A). Consistent with the tropic adhesion of SKBr3 cells to collagen I (FIG. 1K), cells transduced with control Myr-Myc formed numerous and long filopodia when plated on collagen I, and only scarce and short protrusions on plastic, fibronectin, and laminin (FIG. 8). Remarkably, ectopic expression of Myr-oPH-Myc abolished filopodia formation on collagen I entirely (FIG. 8).

To investigate a potential direct link between the obscurin-PH mediated suppression of cell migration and adhesion with filopodia depletion, we treated MDA-MB-231 cells plated on collagen I—the most effective filopodia-inducing ECM protein—with supraphysiological levels of the potent inflammatory mediator bradykinin (R. Kozma et al., Mol Cell Biol, (1995), 15:1942; A. Lepucki et al., J Clin Med, (2022), 11 (5)). Bradykinin treatment increased both the number and length of filopodia in MDA-MB-231 cells transduced with either control Myr-Myc or Myr-oPH-Myc plated on collagen I, compared to vehicle treatment (FIG. 2B). Interestingly, bradykinin rescue of filopodia in cells expressing Myr-oPH-Myc was less effective than vehicle-treated Myr-Myc cells, indicating that bradykinin can partially restore filopodia formation in the presence of the obscurin PH-domain, further underscoring the effectiveness of the obscurin PH-domain (FIG. 2B). Consistent with a direct link between obscurin PH-domain mediated filopodia abrogation and reduced cell migration and adhesion, bradykinin-treated MDA-MB-231 cells expressing Myr-oPH-Myc displayed increased migration (FIG. 2C) and adhesion to collagen I (FIG. 2D), compared to vehicle-treated Myr-oPH-Myc cells.

The obscurin PH-domain suppresses breast cancer cell MMP expression, invadopodia formation, and dissemination

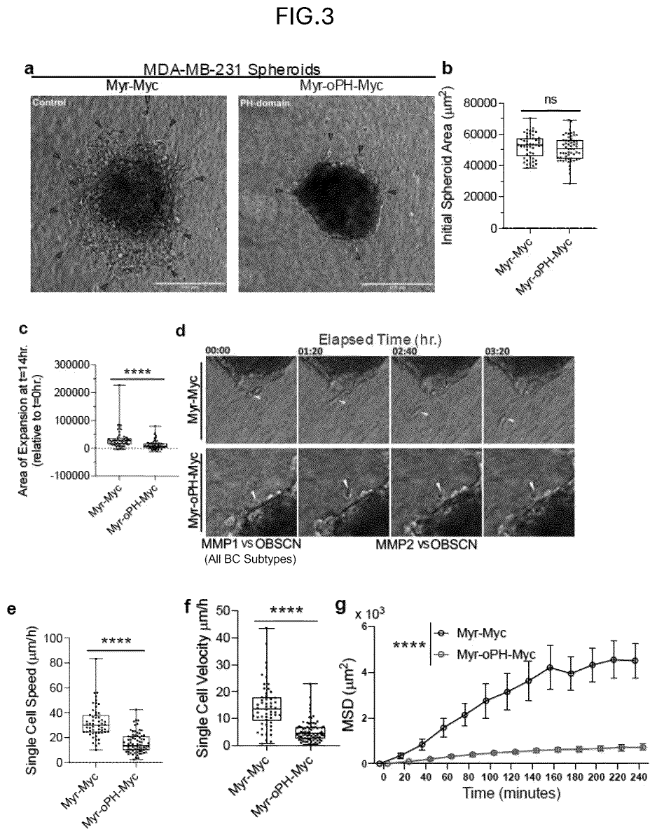

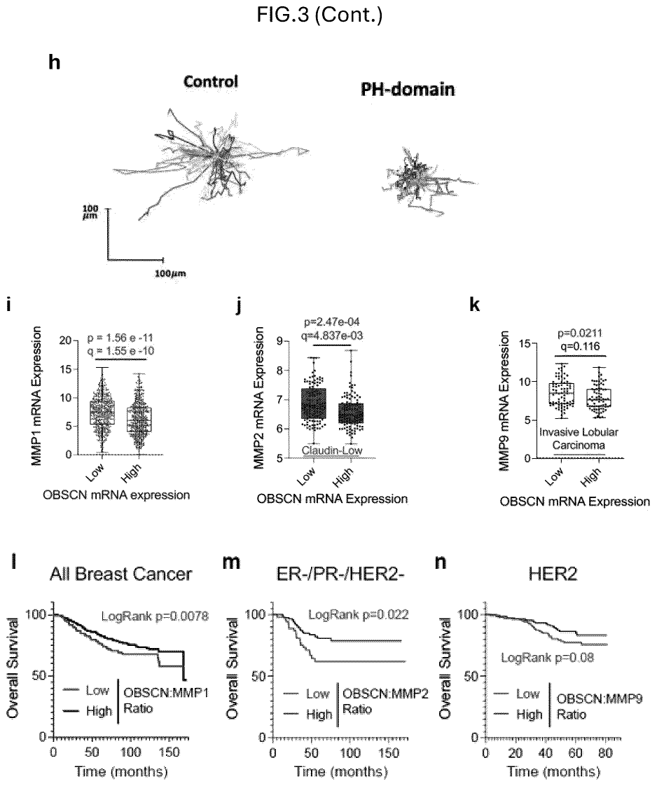

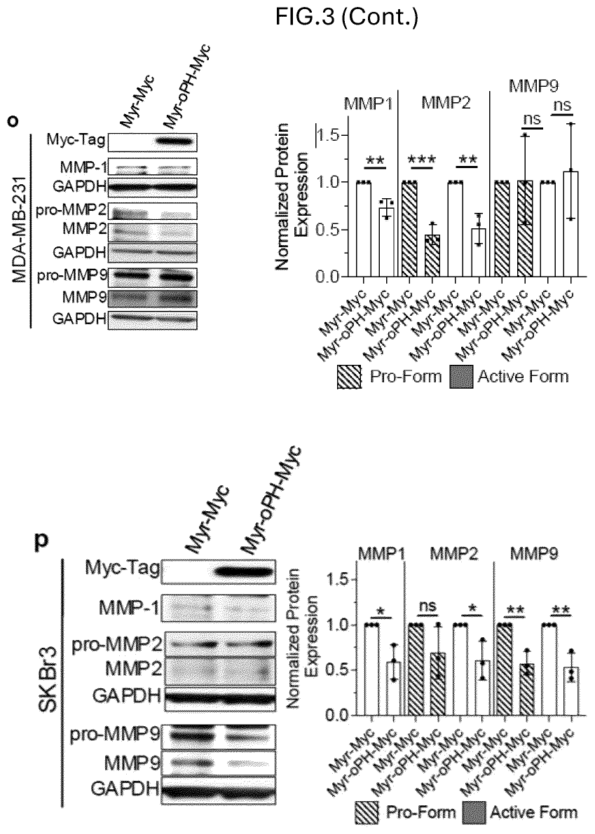

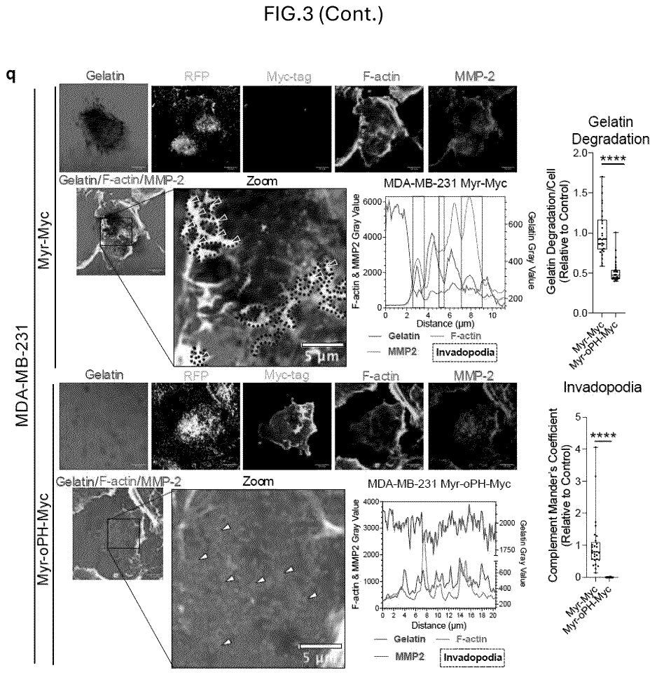

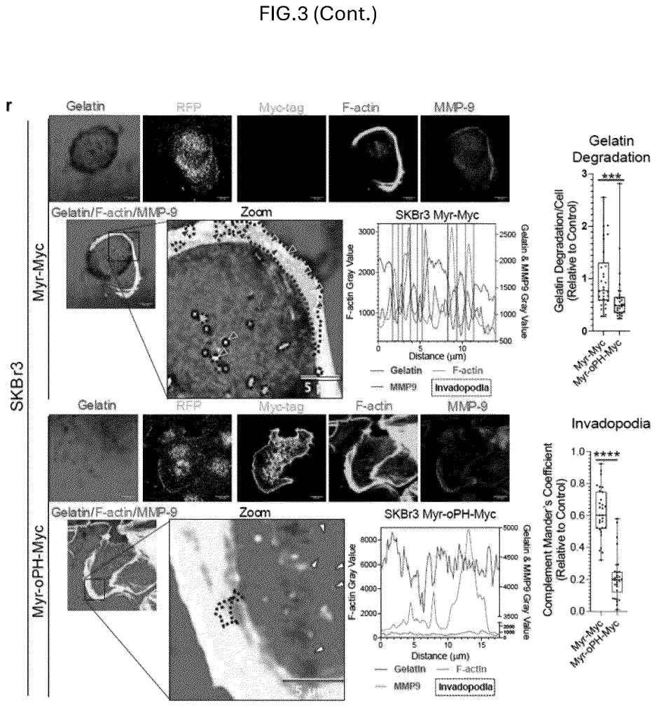

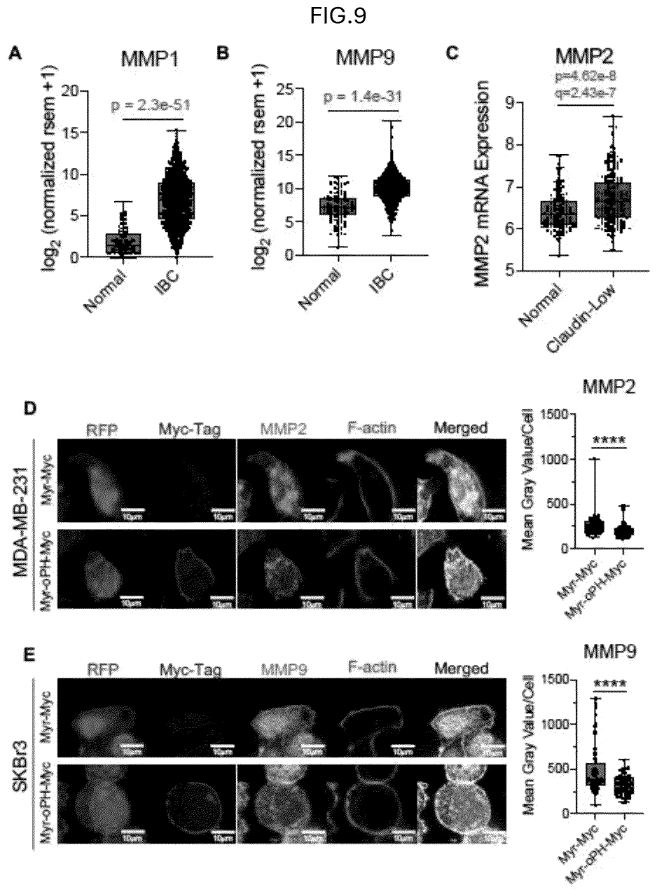

To establish metastatic colonies, disseminating tumor cells are required to invade through the dense ECM microenvironment of the tissue parenchyma (A. W. Lambert et al., Cell, (2017), 168:670). Specifically, invading breast cancer cells express and secrete matrix metalloproteinases (MMP) 1, 2, and 9 downstream of upregulated PI3K/Akt signaling to degrade microenvironmental ECM barriers (R. Zhou et al., Horm Metab Res, (2014), 46:753; Y.-L. K et al., PLoS One, (2017). RNAseq gene expression analysis using the TCGA Wanderer tool (maplab.imppc.org) confirmed markedly increased MMP1 and MMP9 expression in invasive breast tumors, independently of their molecular subtype (FIG. 9A, B), relative to normal breast tissue, whereas evaluation of the Metabric breast cancer dataset using cBioPortal (cBioportal.org) indicated significantly elevated MMP2 expression uniquely in claudin-low breast tumors (FIG. 9C). Considering that obscurin loss drives upregulation of the PI3K/Akt axis, we investigated if human breast tumors displaying reduced obscurin expression contain increased levels of MMP 1, 2, and/or 9 (M. Shriver et al., Oncotarget, (2016), 7:45414; M. Shriver et al., Oncogene, (2015), 34: 4248). Analysis of the 2018 TCGA PanCancer Atlas Invasive Breast Cancer dataset using cBioPortal, including all breast tumor molecular subtypes, revealed a positive correlation between low(er) OBSCN and high(er) MMP1 mRNA levels (FIG. 3A). Moreover, use of the Metabric breast cancer dataset showed a positive correlation between low(er) OBSCN and high(er) MMP2 (FIG. 3B) and MMP9 (FIG. 3C) mRNA levels in claudin-low tumors and invasive lobular carcinomas (ILC), respectively. Relatedly, lower OBSCN:MMP1 ratio correlates with significantly reduced overall survival of breast cancer patients independently of molecular differentiation (kmplot.com; FIG. 3D), while lower OBSCN:MMP2 ratio is linked to markedly decreased overall survival of ER−/PR−/HER2− patients (kmplot.com, comprising the TNBC basal and claudin-low subtypes; FIG. 3E), and lower OBSCN:MMP9 ratio is associated with a trending decline of overall survival of HER2+ patients (kmplot.com; FIG. 3F).

Given these intriguing observations using patient datasets (FIG. 3A-F), we investigated if ectopic expression of the obscurin-PH domain attenuates MMP expression. In agreement with the bioinformatics human data, triple negative/claudin-low MDA-MB-231 cells transduced with Myr-oPH-Myc contained significantly decreased levels of MMP1 and MMP2 compared to cells transduced with control Myr-Myc, whereas MMP9 expression was unaltered (FIG. 3G). Conversely, ILC-like, HER2+ SKBr3 cells transduced with Myr-oPH-Myc expressed markedly reduced levels of MMP1 and MMP9, compared to cells transduced with control Myr-Myc, whereas MMP2 levels were unchanged (FIG. 3H). These findings were further confirmed by immunofluorescence staining of Myr-oPH-Myc expressing MDA-MB-231 and SKBr3 cells cultured on gelatin, which mirrors the 3D microenvironment surrounding breast cancer cells, demonstrating decreased levels of the molecular subtype-specific MMP2 and MMP9, respectively (FIG. 9D, E).

In addition to increasing MMP expression, invading breast cancer cells must traffic MMPs from the Golgi to the tips of PI3K-regulated, actin-based invadopodia to mediate their secretion and subsequent matrix degradation (A. Jacob et al., Front Cell Dev Biol, (2015), 3:4). To assess the ability of the obscurin-PH domain to suppress invadopodia formation, we performed gelatin invadopodia assays combined with immunofluorescent staining to visualize MMP2 and MMP9 positive invadopodia. Remarkably, both MDA-MB-231 (FIG. 3I) and SKBr3 (FIG. 3J) cells transduced with Myr-oPH-Myc displayed a blatant decrease in gelatin degradation compared to their counterparts transduced with control Myr-Myc. To visualize invadopodia, the fluorescent gelatin channel (red) was merged with the F-actin staining (green) to identify F-actin-rich invadopodia within the confines of degraded gelatin space (green structures outlined by dotted black lines), as opposed to non-invadopodia structures positive for F-actin above non-degraded gelatin (yellow structures). Notably, MDA-MB-231 and SKBr3 cells expressing control Myr-Myc formed MMP2 and MMP9 positive invadopodia, respectively, as shown by overlaying MMP2 and MMP9 staining with F-actin-labeled invadopodia (FIG. 3I, J; white structures; blue arrowheads). Quantification of invadopodia abundance, determined as the fraction of F-actin positive protrusions over degraded gelatin space, per cell, indicated that both MDA-MB-231 and SKBr3 cells expressing the obscurin PH-domain extended significantly less invadopodia (FIG. 3I, J). While MMP2 and MMP9 positive puncta (magenta structures; white arrowheads) could still be visualized at the ventral surface of cells expressing Myr-oPH-Myc, these did not colocalize with invadopodia (FIG. 3I, J).

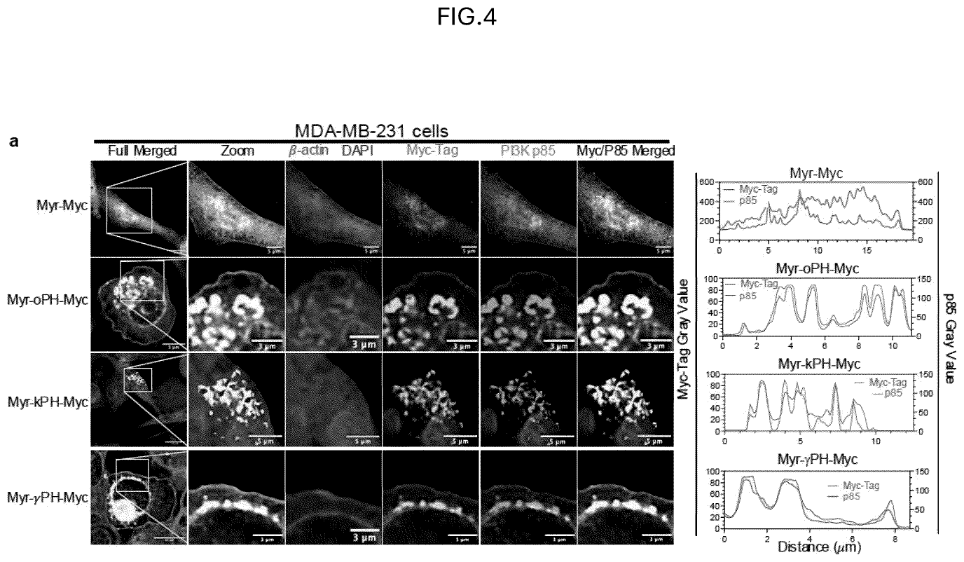

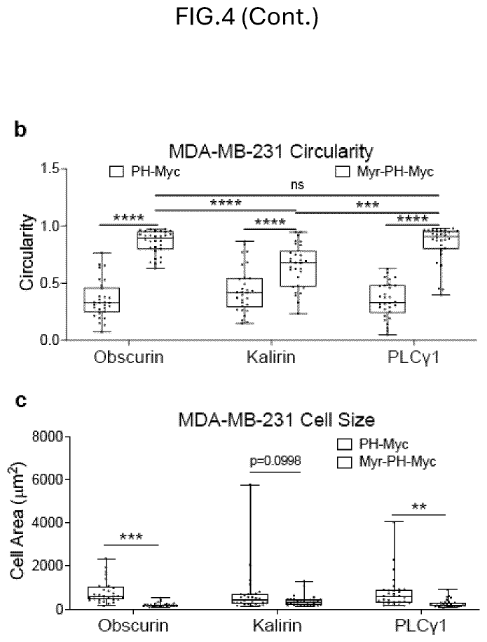

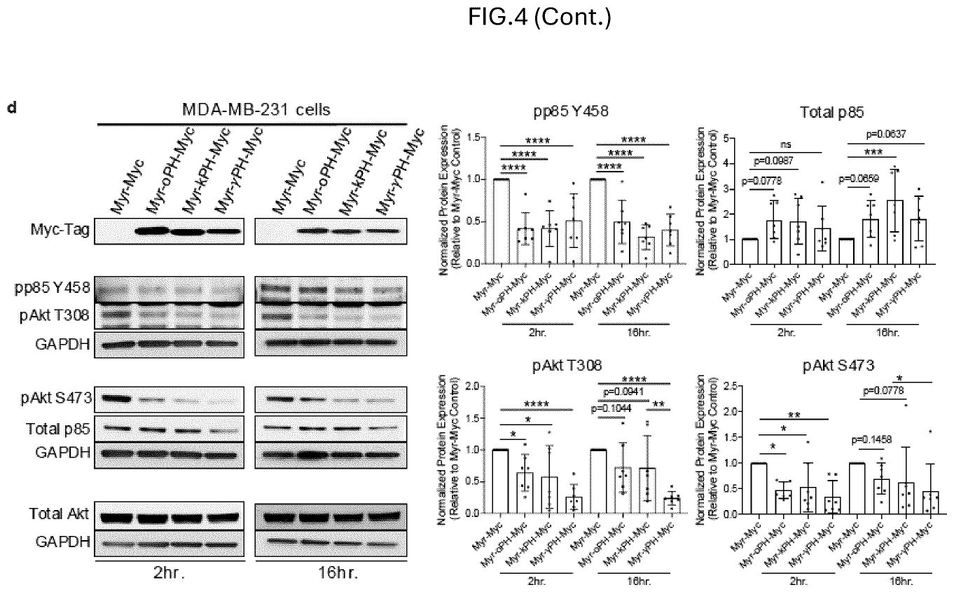

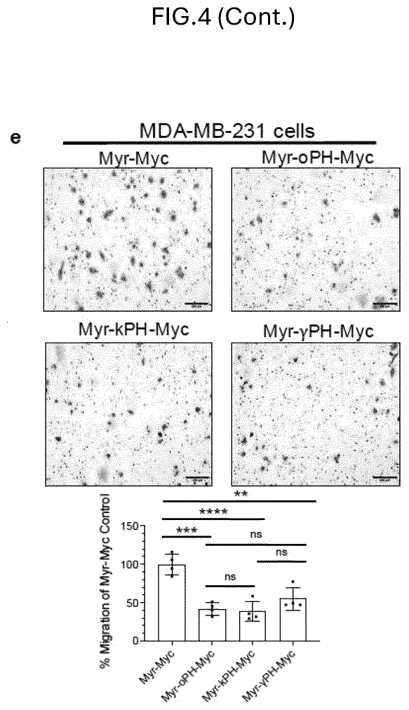

Given the effectiveness of the obscurin-PH domain to suppress breast cancer cell MMP expression and invadopodia formation, we asked whether it impedes invading cell dissemination, too. We recapitulated the extracellular environment of primary breast tumors by forming, embedding, and monitoring the invasive potential of MDA-MB-231 spheroids transduced with Myr-Myc or Myr-oPH-Myc in 3-dimensional (3D) collagen I matrix (FIG. 4A). Excitingly, Myr-oPH-Myc-expressing spheroids displayed significantly reduced cell dissemination (FIG. 4A), as measured by the decreased area of expansion over a period of 18.3 h, compared to Myr-Myc expressing spheroids (FIG. 4B, C). Real-time visualization of the mesenchymal, spindle-like single cells disseminating from the edge of control spheroids confirmed their ability to successfully detach, contrary to the more rounded single cells from obscurin-PH expressing spheroids that failed to do so (FIG. 4D). Single cells disseminating from Myr-oPH-Myc transduced spheroids exhibited markedly reduced speed (FIG. 4E), velocity (FIG. 4F), and mean square displacement (MSD, FIG. 4G) relative to Myr-Myc controls, with the former showing a consistent pattern of dissociation and reassociation with the spheroid edge, whereas the controls maintained a committed and persistent dissemination path (FIG. 4H).

### Nanoparticle Delivery of the Obscurin PH-Domain Suppresses Breast Tumor Growth and Metastasis

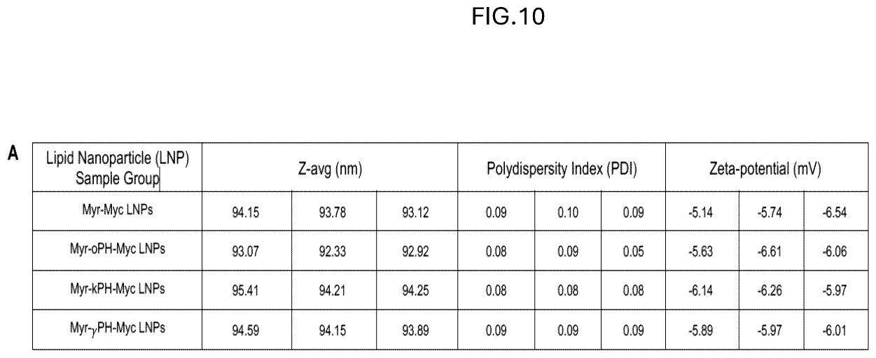

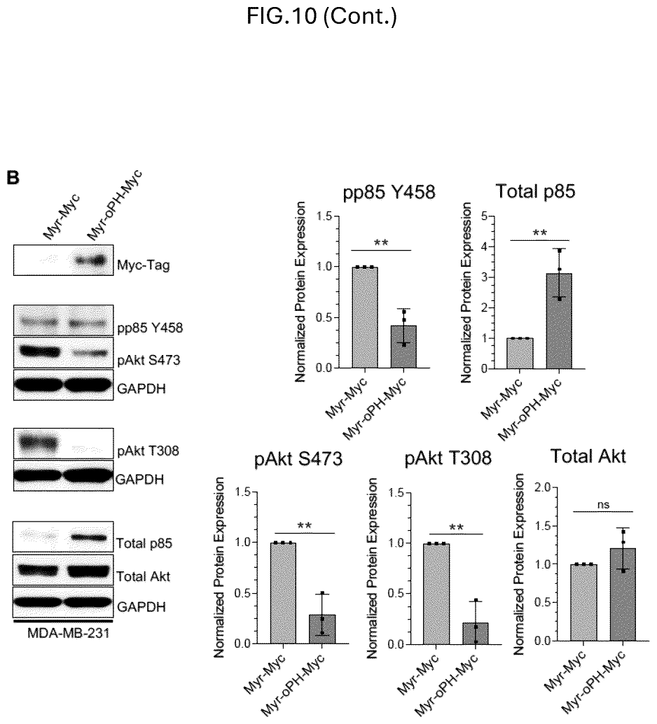

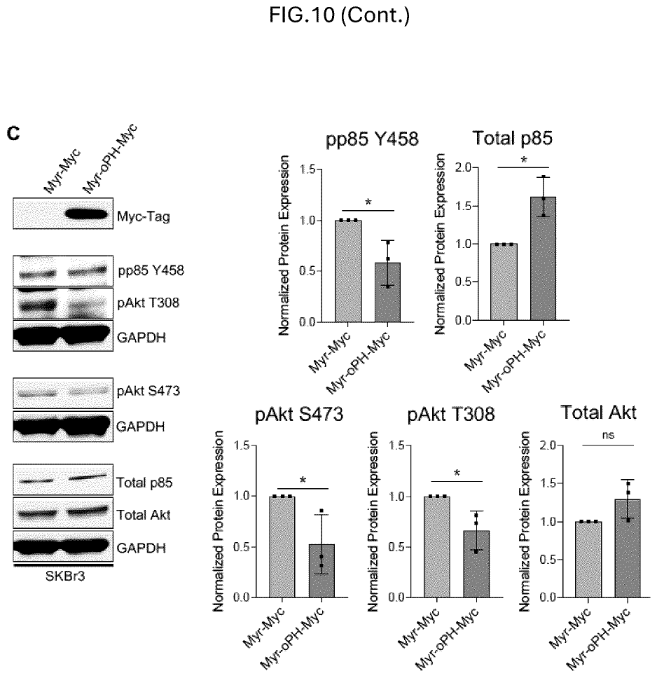

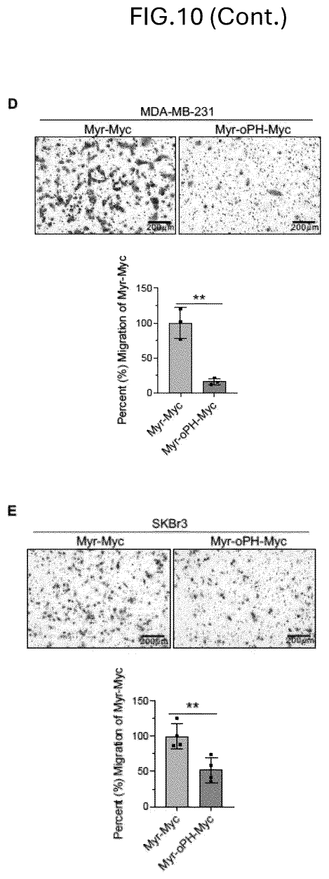

Although gene transfer via adenoviral delivery or transient transfection of plasmid DNA (pDNA) is widely and effectively used for in vitro systems, the potency of either method in vivo is subpar, as they both exhibit poor tumor transduction (M. Souri et al., Mater Today Bio, (2022), 13:100208). We therefore shifted our delivery strategy to the use of lipid nanoparticles (LNPs). Formulated LNPs were optimal for systemic delivery and efficient tumor transfection, as indicated by their appropriate size, polydispersity index (PDI), and zeta potential charge (FIG. 10A) (M. Souri et al., Mater Today Bio, (2022), 13:100208). LNP delivery of Myr-oPH-Myc to MDA-MB-231 and SKBr3 cells markedly decreased PI3K/Akt activity and chemotactic migration (FIG. 10B-E) compared to control Myr-Myc, replicating our findings using adenoviral infection (FIG. 1). Of note, a pronounced upregulation of the total p85 levels was observed (FIG. 10B, C), suggesting a robust sequestration of p85 that likely blocks protein turnover.

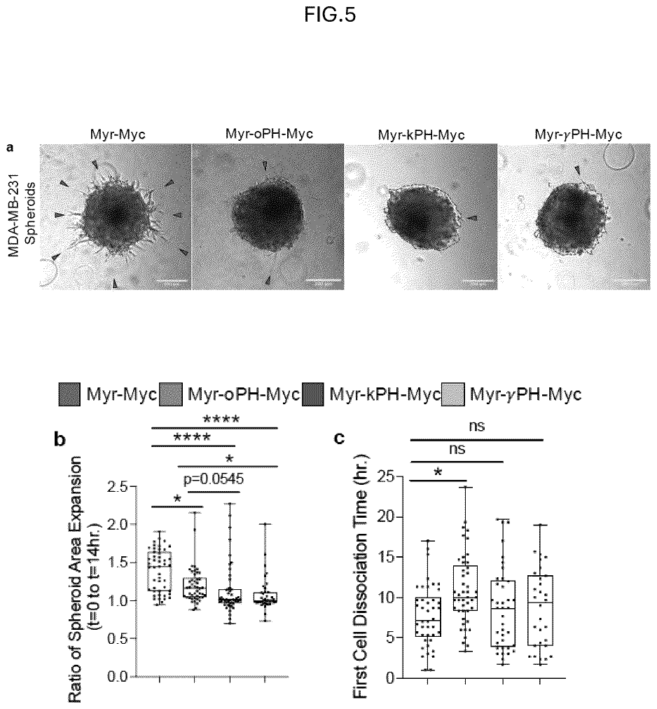

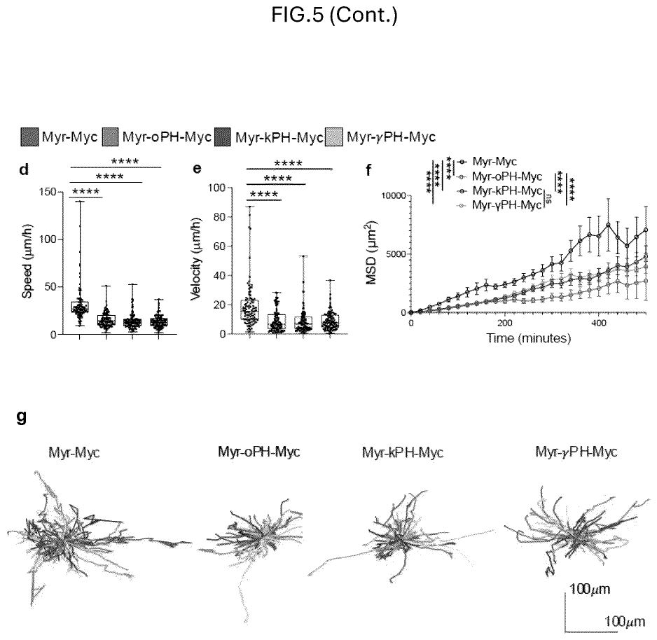

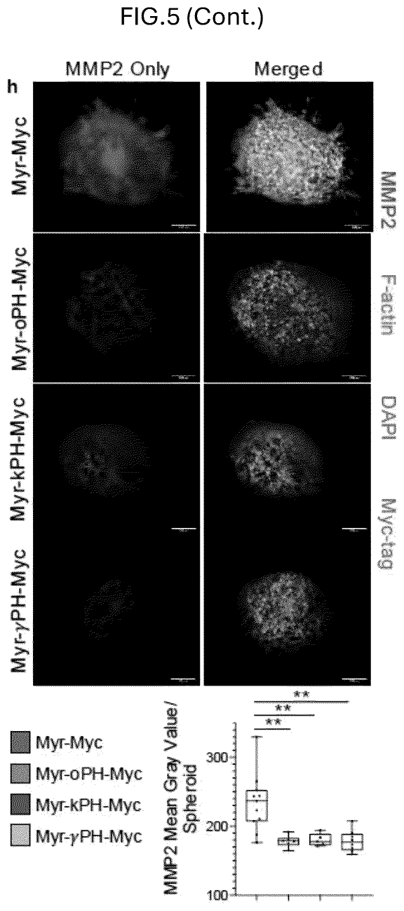

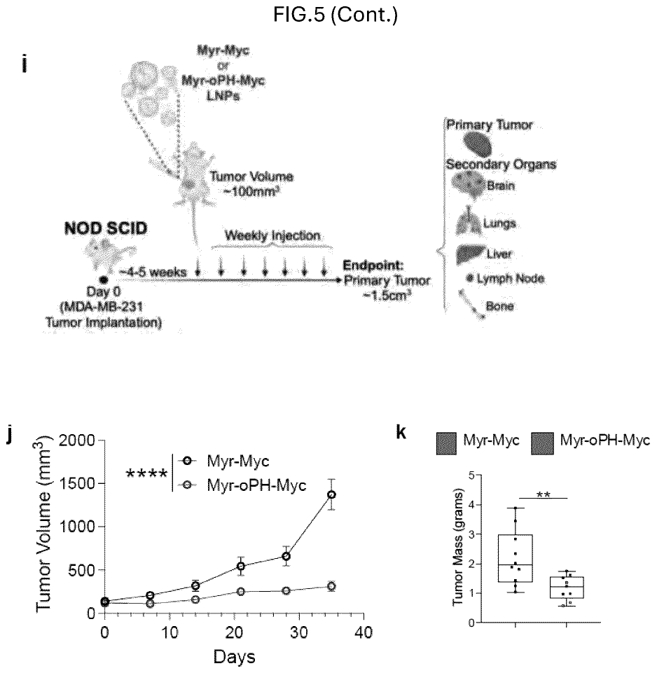

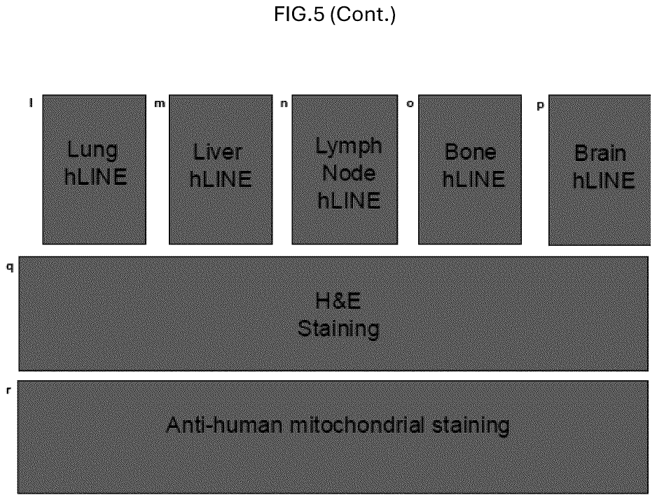

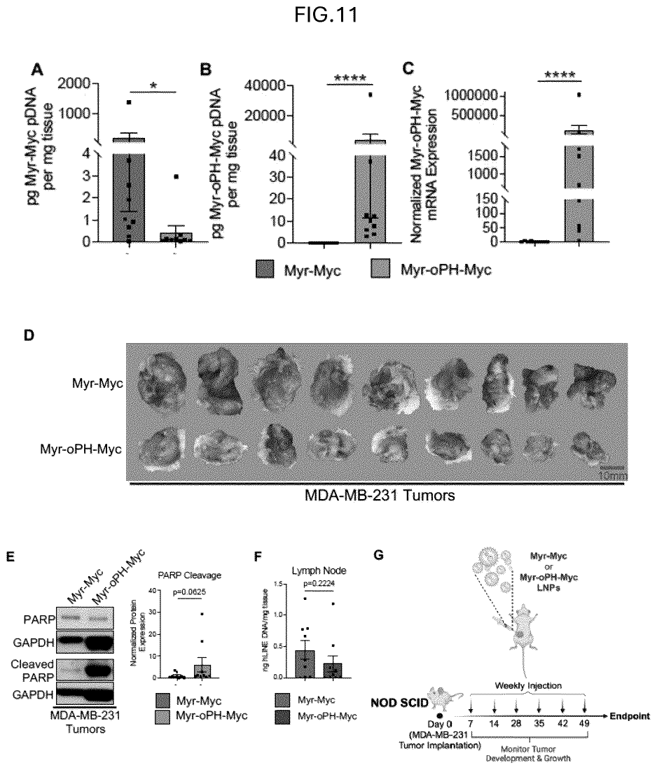

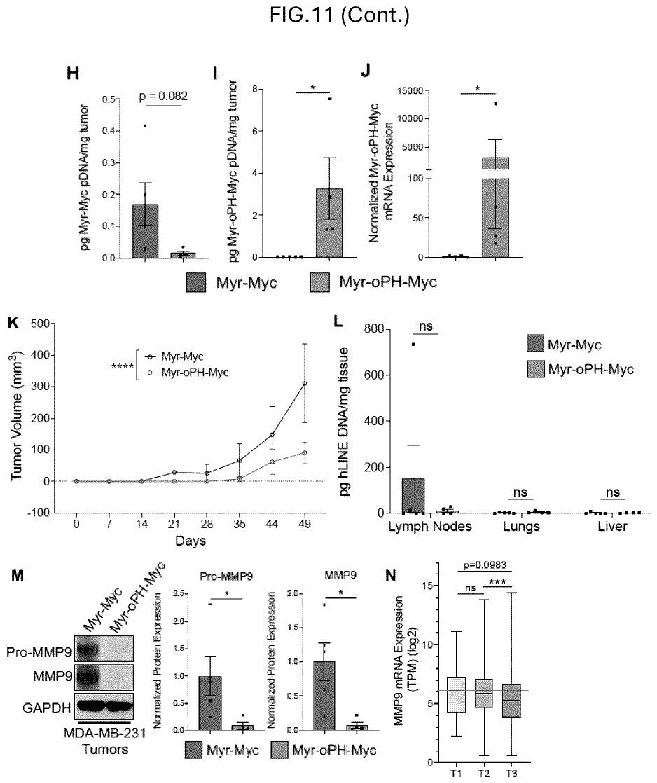

We proceeded to investigate the effect of LNP-delivered Myr-oPH-Myc on tumor growth and metastasis. To simulate breast tumor formation and treatment in patients, MDA-MB-231 cells were implanted orthotopically into the mammary fat pad of NOD SCID mice and allowed to reach a volume of ~100 mm³ before weekly intratumoral injections with either Myr-Myc or Myr-oPH-Myc LNPs for 6 weeks (FIG. 5A). qPCR of primary tumors at endpoint confirmed that Myr-Myc and Myr-oPH-Myc tumors contained Myr-Myc and Myr-oPH-Myc pDNA, respectively (FIG. 11A, B). Likewise, RT-qPCR showed robust expression of the Myr-oPH-Myc transcript in Myr-oPH-Myc transduced tumors (FIG. 11C). Growth measurements over the 6-week injection course indicated that Myr-oPH-Myc LNP-treated breast tumors grew significantly slower (FIG. 5B), reaching an average mass ~50% smaller than Myr-Myc tumors (FIG. 5C, FIG. 11D). In agreement with our in vitro biochemical data, immunoblotting of primary tumors confirmed a significant reduction in the phosphorylation levels of p85-Tyr458 and Akt-Thr308, and a trending decrease in Ser473 phosphorylation levels, indicative of the obscurin PH-domain's suppressive effect on PI3K/Akt activity (FIG. 5D). Notably, PI3K/Akt downregulation was accompanied by a trending increase in PARP cleavage (FIG. 5E) in Myr-oPH-Myc expressing tumors, suggesting enhanced activation of intracellular apoptotic signaling.

More importantly, human long interspersed nuclear element (hLINE) qPCR of secondary organ sites revealed a dramatic reduction in metastasis to the lungs, liver, bone, and brain (FIG. 5E-H) for Myr-oPH-Myc treated mice; however, metastasis to the axillary lymph nodes (FIG. 11F) was variably decreased among Myr-oPH-Myc treated mice. Furthermore, Hematoxylin and Eosin (H&E) staining (FIG. 5I) along with anti-human mitochondria (αhMito) staining (FIG. 5j) and corresponding pathology review (Table Si) of Myr-oPH-Myc lungs revealed near normal tissue parenchyma architecture interspersed with minimal (Severity Score Grade 1) and scattered alveolar microclusters of human breast cancer metastatic cells, contrary to Myr-Myc treated mouse lungs filled with several large (Severity Score Grade 3) alveolar, peribronchial, and peribronchiolar human breast cancer metastatic nodules. Similarly, Myr-oPH-Myc mouse liver contained minimal (Severity Score Grade 1) human breast cancer metastatic microclusters detached from the liver parenchyma, whereas Myr-Myc mouse liver H&E staining (FIG. 5K), αhMito labeling (FIG. 5L), and accompanying pathology review (Table 4) showed sizeable human breast cancer metastatic tumor emboli (Severity Score Grade 2) lodged within feeding portal sinusoids (red asterisks). Interestingly, immunoblotting of Myr-oPH-Myc primary tumors demonstrated robust suppression of MMP1 and MMP2, but a non-significant reduction of MMP9 expression (FIG. 5M), in agreement with our in vitro findings of Myr-oPH-Myc-treated MDA-MB-231 cells grown in 2-dimension (2D) substrata (FIG. 3G).

TABLE 4
Summary of Myr-Myc and Myr-oPH-Myc Mouse Lung and Liver Pathology Report

| LNP | Secondary | Stain | | | | | Tumor | Severity |
|---|---|---|---|---|---|---|---|---|
| Treatment | Organ Site | Type | Stain | H&E Morphology | IHC | Location | Grade | Score |
| Myr-Myc | Lung | H&E | H&E | Multiple nodules, | NA | Lung, alveoli, | High | 3 |
| | | | | alveolar, tumor, poorly | | peribronchial, | | |
| | | | | differentiated, | | peribronchiolar | | |
| | | | | anaplastic, abundant | | | | |
| | | | | cytoplasm, mitoses | | | | |
| | | IHC | Human | NA | Positive | Tumor, | 3+ | |
| | | | Mitochondria | | | cytoplasm | | |
| | | | | | | involving 20- | | |
| | | | | | | 30% of tissue | | |
| Myr-oPH- | Lung | H&E | H&E | Extrinsic, epithelioid | NA | Lung, alveoli | High | 1 |
| Myc | | | | cells, large, anaplastic, | | involving 5% | | |
| | | | | amphophilic cytoplasm, | | of tissue | | |
| | | | | mitoses, inflammation, | | | | |
| | | | | scattered microclusters | | | | |
| | | IHC | Human | NA | Positive | Lung, alveoli | 3+ | |
| | | | Mitochondria | | | involving 5% | | |
| | | | | | | of tissue | | |
| Myr-Myc | Liver | H&E | H&E | Extrinsic, epithelioid | NA | Liver | High | 2 |
| | | | | cells with large signet | | | | |
| | | | | ring cell features, EMH | | | | |
| | | | | scattered, myeloid, | | | | |
| | | | | erythroid, | | | | |
| | | | | megakaryocytes | | | | |
| | | IHC | Human | NA | Positive | Sinusoids, | 3+ | |
| | | | Mitochondria | | | blood, Liver; | | |
| | | | | | | tumor, strong | | |
| | | | | | | cytoplasmic | | |
| | | | | | | involving 10- | | |
| | | | | | | 20% of tissue | | |
| | | | | | | section | | |
| Myr-oPH- | Liver | H&E | H&E | Extrinsic, epithelioid | NA | Liver extrinsic | High | 1 |
| Myc | | | | cells, large, anaplastic, | | detached | | |
| | | | | amphophilic cytoplasm | | microclusters | | |
| | | IHC | Human | NA | Positive | Tumor, | 3+ | |
| | | | Mitochondria | | | cytoplasmic | | |
| | | | | | | involving 5% of | | |
| | | | | | | tissue | | |

To test if the obscurin PH-domain represses MMP9 expression in the initial stages of tumor development, we orthotopically implanted MDA-MB-231 cells into the mammary fat pad of NOD SCID mice, followed by 6 weekly injections of Myr-Myc or Myr-oPH-Myc LNPs, 7 days post-implantation before tumors became palpable (FIG. 11G). qPCR of primary tumors at endpoint confirmed the expression of Myr-Myc and Myr-oPH-Myc pDNA (FIG. 11H, I), and RT-qPCR showed abundant expression of the Myr-oPH-Myc transcript in the respective tumors (FIG. 11J). Similar to the reduced growth of advanced Myr-oPH-Myc treated tumors (FIG. 5B), Myr-oPH-Myc early tumors developed significantly slower and were ~70% smaller compared to Myr-Myc controls (FIG. 11K). Although no metastatic burden was detected (or expected) at endpoint via hLINE DNA quantitation (FIG. 11L) in either Myr-Myc or Myr-oPH-Myc tumors given their early stage and small size (~300 mm³ and ~90 mm³, respectively), immunoblotting revealed significant suppression of MMP9 expression (FIG. 11M). Consistent with this finding, cBioPortal analysis of invasive breast carcinoma patient samples from the 2024 TCGA and Genomic Data Commons dataset confirmed that MMP9 expression is higher in T1/T2 early-stage samples relative to T3 later-stage samples (FIG. 11N). Thus, Myr-oPH-Myc LNP-treatment induces a transient decrease in MMP9 levels during early tumorigenesis, while maintaining a prominent suppression of MMP1 and MMP2 expression in later stages.

### The Kalirin and PLCγ1 PH-Domains Suppress PI3K/Akt Activity, Cell Migration, and Dissemination

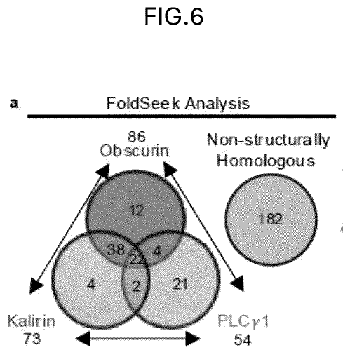

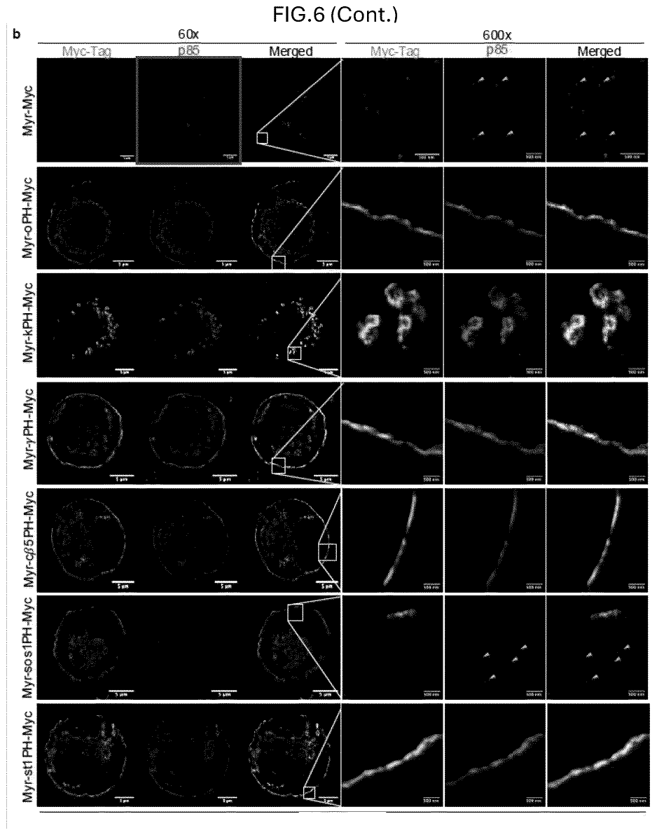

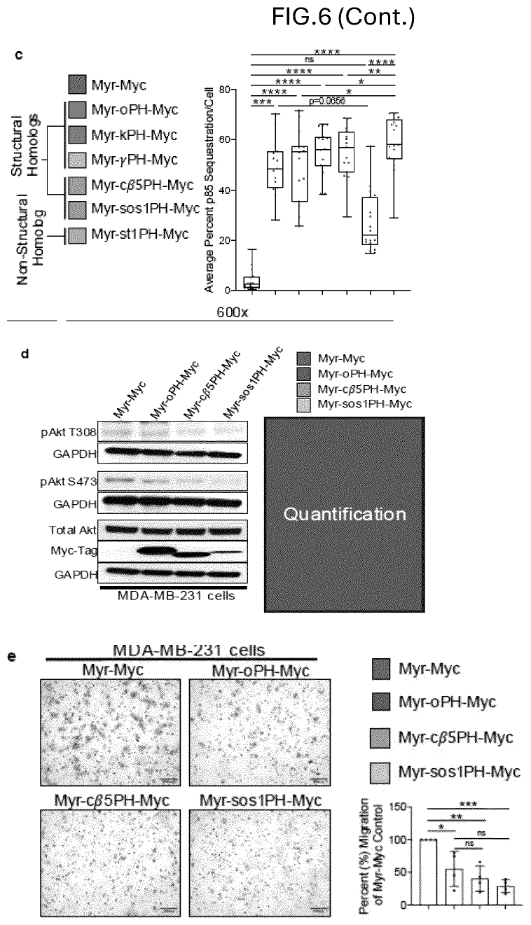

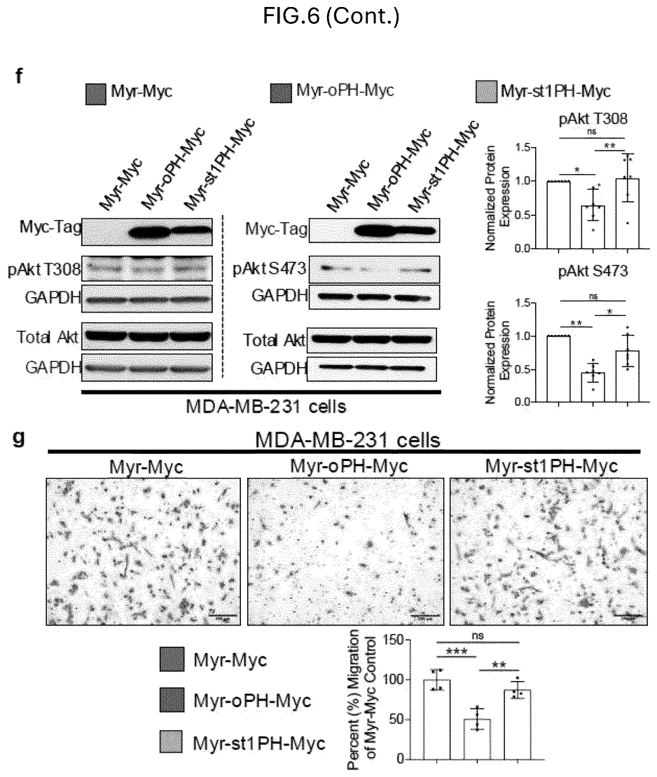

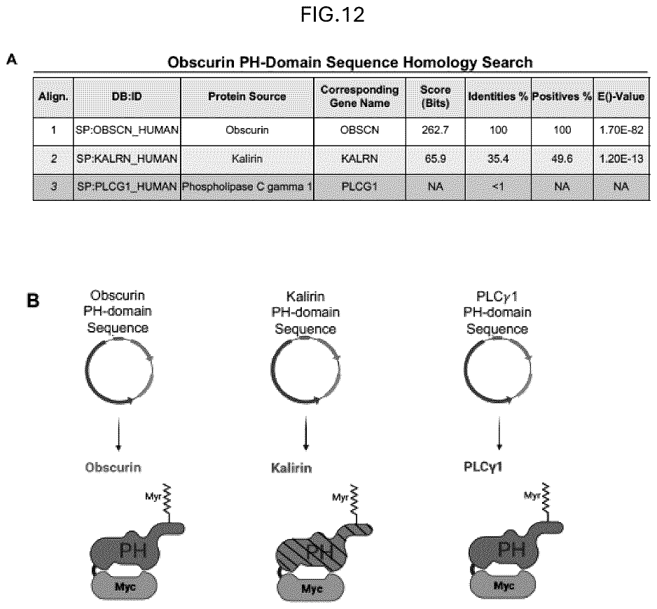

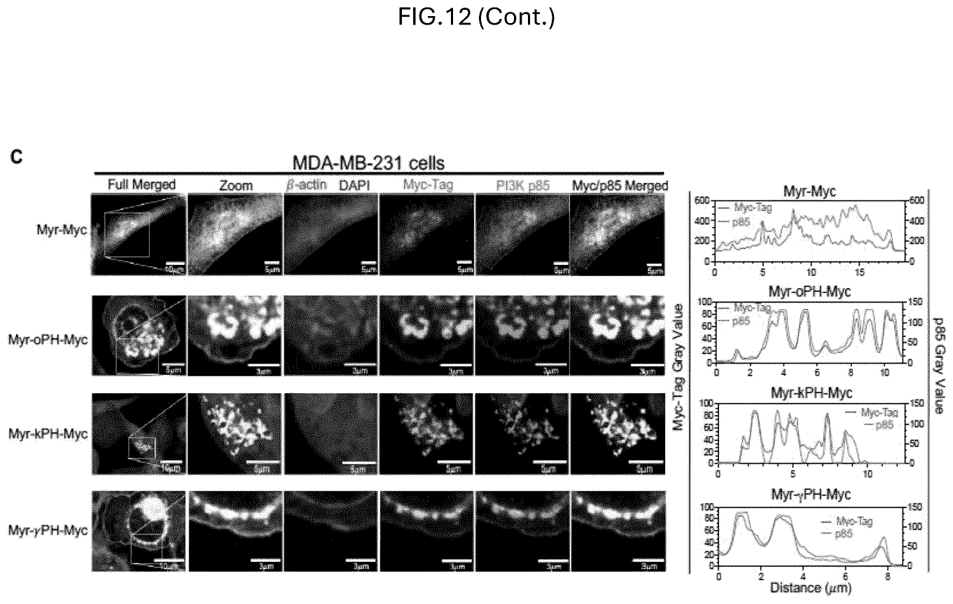

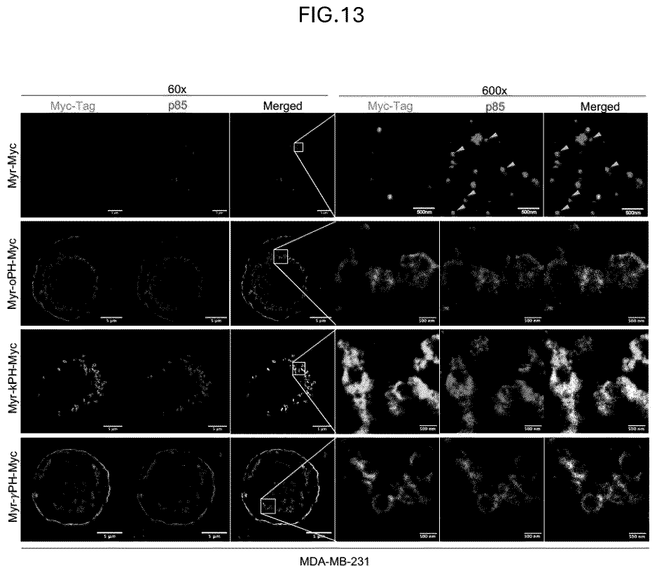

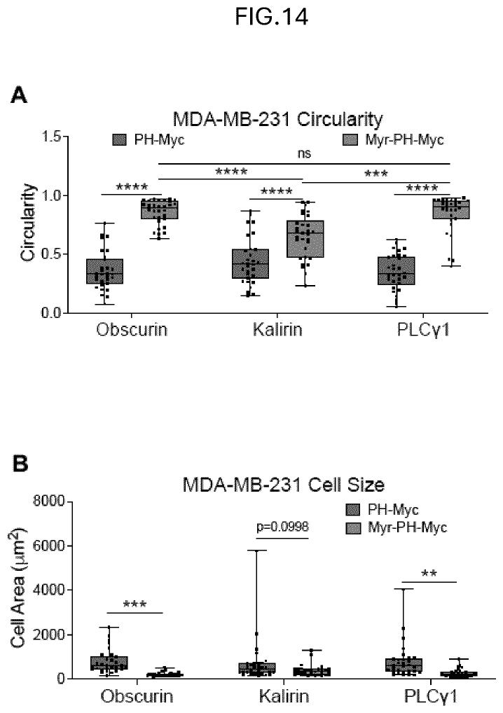

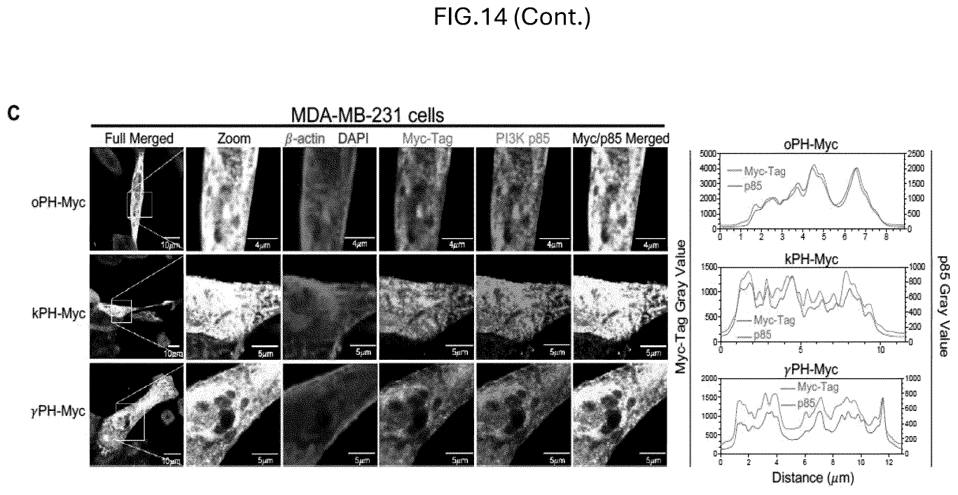

Given the potent anti-metastatic properties of the obscurin-PH domain, we examined if other PH-domains possess similar capabilities. Using an NCBI blast search, we identified that the kalirin PH-domain (kPH) possesses the highest sequence identity (35.4%) with the obscurin PH-domain, while the phospholipase-Cγ1 (PLCγ1) PH-domain (γPH) exhibits minimal sequence homology (<1%; FIG. 12A). Following the generation of myristoylated forms of the kalirin-(Myr-kPH-Myc) and PLCγ1 (Myr-γPH-Myc) PH-domains (FIG. 12B), we examined their localization using transient transfection of the respective pDNA constructs in MDA-MB-231 cells. While Myr-kPH-Myc selectively concentrated to perinuclear vesicles, Myr-γPH-Myc targeted to the cell membrane and the perinuclear space, similar to Myr-oPH-Myc (FIG. 12C). On the other hand, Myr-Myc protein maintained a faint and diffuse distribution. Notwithstanding their distinct distribution patterns, all three PH-domains effectively sequestered PI3K-p85 to their respective subcellular locations, whereas Myr-Myc protein did not (FIG. 12C). The sequestration of p85 was further substantiated by Stimulated Emission Depletion (STED) microscopy, which enabled visualization of PH-domain/p85 spatial localization at a 30 nm resolution. Consistent with our findings using confocal optics, Myr-oPH-Myc, Myr-kPH-Myc, and Myr-γPH-Myc reside in close proximity with p85 at the cell membrane (FIG. 6A) and/or perinuclear vesicles (FIG. 13), indicative of their association; in contrast, Myr-Myc protein maintains a diffuse punctate distribution that does not overlap with p85. Quantification of the proximal distribution of the individual PH-domains and p85 revealed that greater than or equal to 50% of p85 was sequestered to the cell membrane and/or intracellular vesicles, compared to less than 10% in the presence of Myr-Myc (FIG. 6B). Notably, all three myristoylated PH domains promoted cell circularity (FIG. 14A) and diminished cell area (FIG. 14B) compared to their non-myristoylated counterparts, as demonstrated by β-actin staining (FIG. 14C), which are hallmarks of indolent, non-migratory cells with poor metastatic propensity (X. Lu et al., Cell Res, (2010), 20:1012). These findings implicate that membrane targeting of all three PH-domains elicits similar anti-metastatic cell morphology changes.

Akin to Myr-oPH-Myc, transient overexpression of Myr-kPH-Myc and Myr-γPH-Myc in MDA-MB-231 cells significantly inhibited PI3K/Akt activity 2 h post-serum challenge, as shown by diminished phosphorylation of p85-Tyr458 and Akt-Thr308/Ser473, while total levels of p85 and Akt remained unaltered (FIG. 6C). Consistent with the decreased PI3K/Akt activity, MDA-MB-231 cells expressing either Myr-kPH-Myc or Myr-γPH-Myc exhibited reduced chemotactic migration in a Transwell assay, thereby replicating the effects observed following ectopic expression of Myr-oPH-Myc (FIG. 6D). Relatedly, LNP delivery of Myr-kPH-Myc or Myr-γPH-Myc in MDA-MB-231 spheroids embedded in 3D collagen I effectively suppressed cell dissemination similar to Myr-oPH-Myc (FIG. 6E). This was evidenced by a notable reduction in the area of spheroid expansion over a period of 14 h, along with decreased speed, velocity, and mean square displacement of disseminating single cells relatively to Myr-Myc control spheroids (FIG. 6F-J). Consistent with these findings, immunostaining of all three PH-transduced spheroids for MMP2 revealed drastically diminished expression compared to Myr-Myc controls (FIG. 6K).

Molecular Modeling of PH Domain Binding to the p85-SH3 Domain

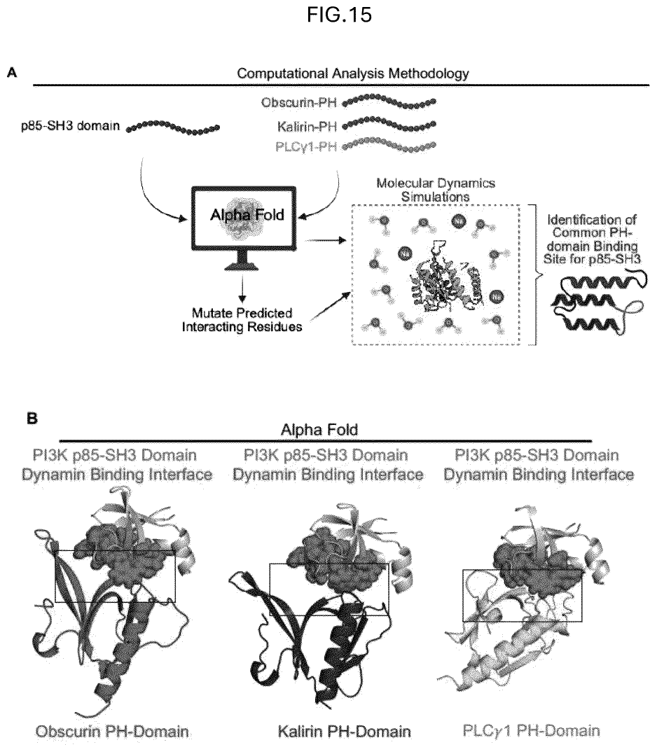

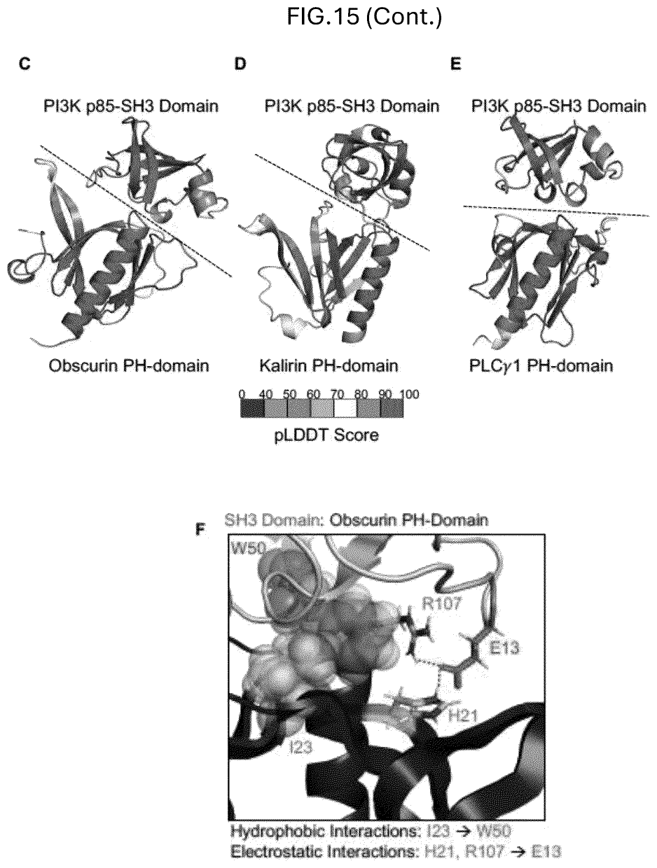

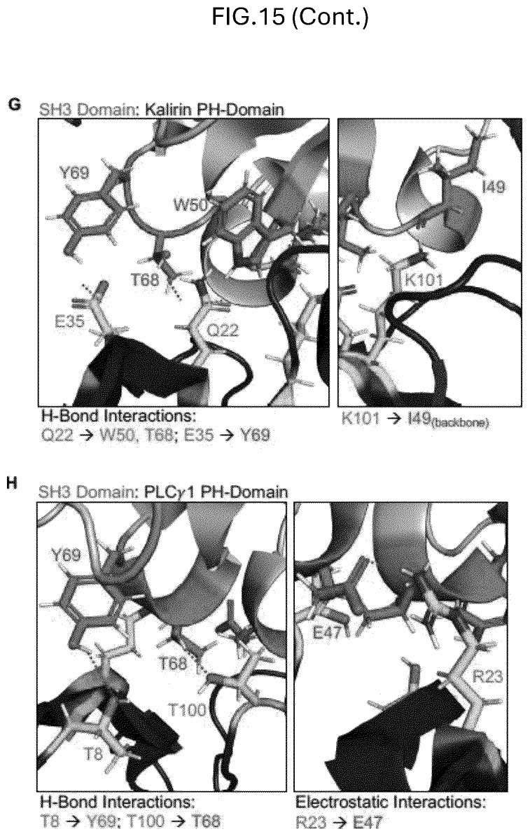

Our findings indicate that the obscurin, kalirin, and PLCγ1 PH-domains can efficiently sequester p85 to block breast cancer cell dissemination and metastasis, while prior mechanistic work has established that the obscurin-PH domain directly binds to the SH3-domain of the p85 subunit with high affinity (M. Shriver et al., Oncotarget, (2016), 7:45414). To gain insights about the structural binding interface of the PH and SH3 domains, we input each PH-domain sequence into alpha-fold and simulated binding with the p85-SH3 domain (FIG. 15A). Although amino acid similarity among the three PH-domains varies from ~34% to <1%, all three PH-domains folded in a structurally analogous manner and interlocked with the dynamin-binding interface of the p85-SH3 domain, which is the canonical protein-binding region of p85-SH3, thereby generating confidence in the validity of this model (FIG. 15B) (G. W. Booker et al., Cell, (1993), 73:813). All models were of high quality, with predicted local distance difference test (pLDDT) values of 89.8, 85.2, and 90.7 for the obscurin, kalirin, and PLCγ1 PH-domains, respectively (FIG. 15C-E); of note, high quality pLDDT values range between 80-100 (K. Tunyasuvunakool et al., Nature, (2021), 596:590). Furthermore, all three PH-domains remained bound to the p85-SH3 domain during each molecular dynamics simulation (50-120 ns) without significant change to the binding orientation, suggesting a stable interaction.

Given the marked similarity across the three equilibrated PH:SH3 complexes, we next inquired about potential residues that facilitate binding. In all three PH-domains, we identified multiple residues that interlocked with a hydrophobic pocket formed by SH3 at the dynamin-binding interface (FIG. 15F-H). To test the contribution of those interactions, we performed in silico mutagenesis of various contributing PH-domain residues, as predicted by alpha-fold, with the intention of destabilizing the PH:SH3 complex. Bulky hydrophobic amino acids participating in relevant interactions or hydrophilic amino acids participating in hydrogen bonds were substituted with the smaller hydrophobic alanine (A), whereas positively charged amino acids participating in electrostatic interactions were substituted with negatively charged glutamic acid (E).

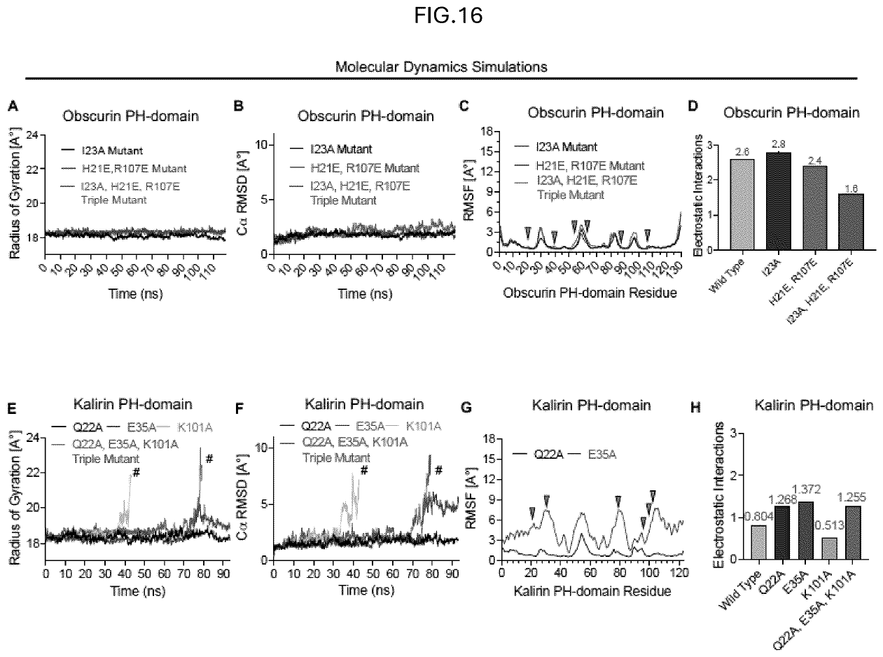

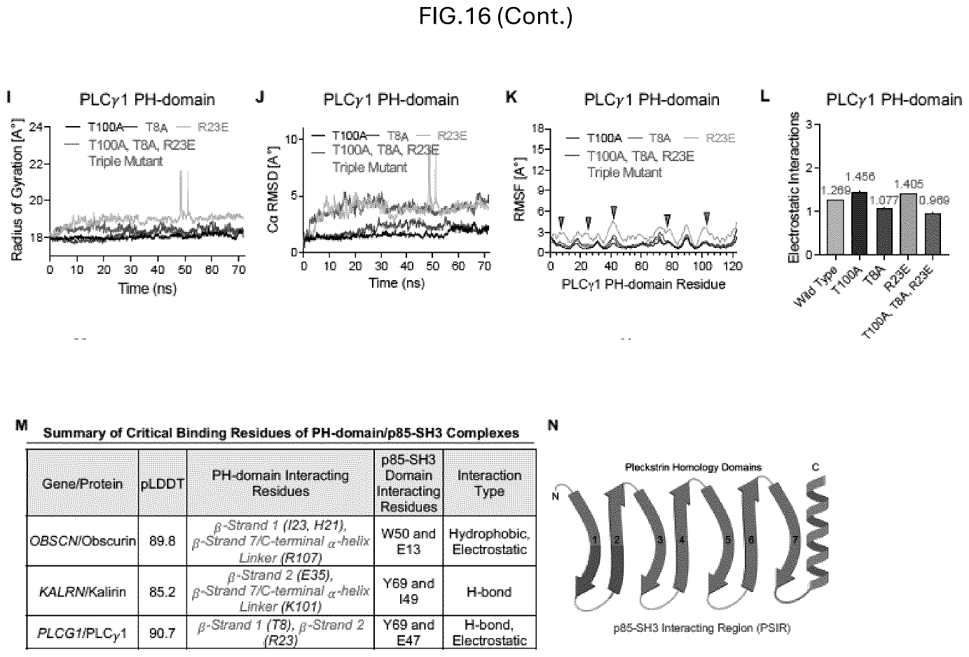

Isoleucine-23 (I23) of β-strand 1 in the obscurin PH-domain orchestrates hydrophobic interactions with tryptophan-50 (W50) in the SH3 domain, while histidine-21 (H21) of β-strand 1 and arginine-107 (R107) of the β-strand 7/C-terminal α-helix linker (β-7 linker) in the obscurin-PH domain coordinate electrostatic interactions with glutamic acid-13 (E13) of the SH3 domain (FIG. 15F). Modeled mutagenesis of I23A, H21E, and R107E combinatorially induced significant motion only in the presence of the triple-mutant, as measured by a larger radius of gyration (R_{g}; FIG. 16a). Furthermore, the increased overall root mean square deviation (RMSD; FIG. 16B), higher root mean square fluctuation (RMSF; FIG. 16C), and loss of stabilizing electrostatic interactions (FIG. 16D) collectively indicate enhanced residue motion involved in complex formation. The need to induce multiple mutations to adequately disrupt the complex suggests a robust obscurin-PH/p85-SH3 interaction, congruent with their experimentally determined K_{D} of ~50 nM (M. Shriver et al., Oncotarget, (2016), 7:45414).

In comparison, models of the kalirin and PLCγ1 PH-domains complexed with the p85-SH3 domain were more susceptible to dissociation when mutations of critical residues were modeled. Residues in analogous locations to those of the obscurin PH-domain were predicted to support complex formation, yet with distinct variations. For instance, the kalirin-PH/p85-SH3 complex is dominated by hydrogen bonds between the kalirin-PH glutamine-22 (Q22) of β-strand 1, E35 of β-strand 2, and lysine-101 (K101) of the β-7 linker to the p85-SH3 W50, threonine-68 (T68), tyrosine-69 (Y69), and 149, respectively (FIG. 15G). In silico mutagenesis of these kalirin-PH residues predicted that K101A, but not Q22A or E35A, induces complete dissociation of the kalirin-PH domain from p85-SH3 due to a profound increase in R_{g}, RMSD, and RMSF, as well as a considerable loss in electrostatic interactions (FIG. 16E-H). Interestingly, an increase in the total number of interdomain electrostatic interactions in both the Q22A and triple-mutant (Q22A/E35A/K101A) suggested that the polar Q22 hinders stable binding of the wild-type, as the potent destabilizing effect of the K101A single mutant is masked by the addition of Q22A in the triple mutant (FIG. 16H). Moreover, T8 in β-strand 1 and T100 in the β-7 linker of the PLCγ1-PH domain form hydrogen bonds with Y69 and T100 of the p85-SH3 domain, respectively, while the PLCγ1-PH R23 of β-strand 2 forms an electrostatic interaction with the p85-SH3 E47 (FIG. 15H). Mutagenesis modeling of PLCγ1-PH R23E destabilized the complex, as seen by increased R_{g}, RMSD, and RMSF values (FIG. 16I-K) at the binding interface. Likewise, T8A also decreased total electrostatic interactions and somewhat destabilized the PLCγ1-PH/p85-SH3 interaction interface (FIG. 16L).

Taken together, the obscurin-PH/p85-SH3 complex is predicted to demonstrate greater overall stability compared to that of the kalirin-PH/p85-SH3 and the PLCγ 1-PH/p85-SH3, as determined by the minimal complex disruption induced by the triple mutant obscurin-PH compared to the blatant complex dissociation using single mutants for kalirin and PLCγ1 PH-domains. Importantly, our simulated mutagenesis of select residues across all three PH-domains further predicted that disruptive substitutions in the latter half of β-strand 1, the first half of β-strand 2, and the β-7 linker, destabilize interactions with either W50 or Y69 of p85-SH3 that orchestrate PH-SH3 binding (FIG. 16M). Consequently, we jointly refer to the latter half of β-strand 1, the first half of β-strand 2, and the β-7 linker of a PH-domain as the p85-SH3 interacting region, PSIR (FIG. 16N), found in PH-domains with p85 binding and sequestering capabilities.

### Conclusions

Collectively, our work unveils a previously elusive class of non-chemical PI3K inhibitors in the form of the PH-domain with potent anti-metastatic properties, addressing an urgent unmet clinical need. The mechanism by which obscurin-PH blocks breast cancer metastasis is multifaceted; our unique construct quenches PI3K catalytic activity to dampen downstream PI3K/Akt signaling, rewires cytoskeletal actin polarity to block filopodia and invadopodia formation, and silences invasive MMP expression. Critically, we put forth not one, but three variants of this novel class of PI3K inhibitors, leveraging their unique p85-sequestration capabilities through the structurally conserved PSIR, thereby tripling our discovery of novel anti-metastatic agents. Together, these findings pave the way for new targeted therapies to counter breast cancer metastatic progression and improve patient survival.

### Experimental Section/Methods

### Human Cell Lines and Cell Culture

MCF10A (RRID:CVCL_0598), MDA-MB-231 (RRID:CVLC_0062), and SKBr3 (RRID:CVLC_0033) cells were purchased from ATCC (Manassas, Virginia). MCF10A cells were cultured as previously described (M. Shriver et al., Oncotarget, (2016), 7:45414). MDA-MB-231 cells were maintained in DMEM supplemented with 10% FBS and 1% penicillin-streptomycin, while SKBr3 cells were maintained in McCoys 5A Modified Medium supplemented with 10% FBS and 1% penicillin-streptomycin. All cells were cultured in 5% CO2 at 37° C. in a humidified tissue culture incubator. Cells were routinely checked for Mycoplasma contamination using the MycoGuard Mycoplasma PCR Detection Kit (Genecopoeia, Rockville, MD), according to the manufacturer's protocol, and were confirmed to be free of contamination. All cells were used between passages 8 and 22.

### Cloning and Construct Generation

Myr-Myc Construct: The myristoylation (Myr) sequence (5′-ATGGGAAGCAGCAAGAGCAAGCCAAAG-3′) (SEQ ID NO:8) was ligated into the pCMV6-Entry Mammalian Expression Vector (OriGene, Rockville, MD) as annealed overlapping oligos (Table 5) at AscI and XhoI sites (NEB, Ipswich, MA), in frame with the COOH-terminal Myc and DDK tags and transformed into E. coli cells. The Myr-Myc sequence was then PCR-amplified using OneTaq 2× Master Mix (NEB, NEB, Ipswich, MA) and ligated into the pCMV6-Entry Mammalian Expression Vector (OriGene, Rockville, MD) at AscI and PmeI sites (NEB, Ipswich, MA), introducing a stop codon prior to the DDK tag, to eliminate it. The pCMV6-Myr-Myc plasmid was subsequently gel-purified using the QIAquick Gel Extraction Kit (Qiagen, Germantown, MD) and transformed into E. coli cells.

TABLE 5
Pleckstrin Homology Domain Construct Primer
Sequences for Cloning

| | Forward or | |
|---|---|---|
| | Reverse | |
| Primer | Primer | Primer sequence (5′→3′) |
| Myr-Myc-DDK | Forward | CGCGCCATGGGAAGCAGCAAG |
| | | AGCAAGCCAAAGC (SEQ ID |
| | | NO: 9) |
| Myr-Myc DDK | Reverse | TCGAGCTTTGGCTTGCTCTTGCT |
| | | GCTTCCCATGG |
| | | (SEQ ID NO: 10) |
| Myr-Myc | Forward | TAAGCAGGCGCGCCATGGGAA |
| | | GCAGCAAGAGCAAGCCAAAG |
| | | (SEQ ID NO: 11) |
| Myr-Myc | Reverse | TGCTTAGTTTAAACTTACAGAT |
| | | CCTCTTCTGAGATGAGTTTCTGC |
| | | TC (SEQ ID NO: 12) |
| OBSCN PH- | Forward | TAAGCAGGCGCGCCATGCTCAT |
| Myc-DDK | | GGAGAACTACCCAGGCACC |
| | | (SEQ ID NO: 13) |
| OBSCN PH- | Reverse | TGCTTACTCGAGCAGACGCTGC |
| Myc-DDK | | TGGATGCCACA |
| | | (SEQ ID NO: 14) |
| OBSCN PH-Myc | Forward | TAAGCAGGCGCGCCATGCTCAT |
| | | GGAGAACTACCCAGGCACC |
| | | (SEQ ID NO: 15) |
| OBSCN PH-Myc | Reverse | TGCTTAGTTTAAACTTACAGAT |
| | | CCTCTTCTGAGATGAGTTTCTGC |
| | | TC (SEQ ID NO: 16) |
| OBSCN Myr- | Forward 1 | GCAGCAAGAGCAAGCCAAAGC |
| PH-Myc | | TCATGGAGAACTACCCAGGCAC |
| | | C (SEQ ID NO: 17) |
| OBSCN Myr- | Forward 2 | TAAGCAGGCGCGCCATGGGAA |
| PH-Myc | | GCAGCAAGAGCAAGCCAAAG |
| | | (SEQ ID NO: 18) |
| OBSCN Myr- | Reverse | TGCTTAGTTTAAACTTACAGAT |
| PH-Myc | | CCTCTTCTGAGATGAGTTTCTGC |
| | | TC (SEQ ID NO: 19) |
| KALRN PH-Myc | Forward | CTAGATGGCGCGCCATGCTGGA |
| DDK | | AGGCTTTGATGAAAAC |
| | | (SEQ ID NO: 20) |
| KALRN PH-Myc | Reverse | TAGCCTCTCGAGAATGCGTTCC |
| DDK | | TGAATCACTTCGCG (SEQ ID |
| | | NO: 21) |
| KALRN PH-Myc | Forward | CTAGATGGCGCGCCATGCTGGA |
| | | AGGCTTTGATGAAAAC |
| | | (SEQ ID NO: 22) |
| KALRN PH-Myc | Reverse | TGCTCAGTTTAAACTTACAGAT |
| | | CCTCTTCTGAGATGAGTTTCTGC |
| | | TC (SEQ ID NO: 23) |
| KALRN Myr- | Forward 1 | GCAGCAAGAGCAAGCCAAAGA |
| PH-Myc | | TGCTGGAAGGCTTTGATGAAAA |
| | | C (SEQ ID NO: 24) |
| KALRN Myr- | Forward 2 | TAAGCAGGCGCGCCATGGGAA |
| PH-Myc | | GCAGCAAGAGCAAGCCAAAG |
| | | (SEQ ID NO: 25) |
| KALRN Myr- | Reverse | TGCTCAGTTTAAACTTACAGAT |
| PH-Myc | | CCTCTTCTGAGATGAGTTTCTGC |
| | | TC (SEQ ID NO: 26) |
| PLCG1 PH-Myc | Forward | CTAGATGGCGCGCCATGCTGGA |
| | | AGTGGGCACCGTGATG |
| | | (SEQ ID NO: 27) |
| PLCG1 PH-Myc | Reverse | TGCTCAGTTTAAACTTACAGAT |
| | | CCTCTTCTGAGATGAGTTTCTGC |
| | | TC (SEQ ID NO: 28) |
| PLCG1 Myr- | Forward | TAAGCAGGCGCGCCATGGGAA |
| PH-Myc | | GCAGCAAGAGCAAGCCAAAG |
| | | (SEQ ID NO: 29) |
| PLCG1 Myr- | Reverse | TGCTCAGTTTAAACTTACAGAT |
| PH-Myc | | CCTCTTCTGAGATGAGTTTCTGC |
| | | TC (SEQ ID NO: 30) |

Obscurin Pleckstrin Homology (PH)-Domain constructs: The obscurin PH-domain was cloned from cDNA reverse transcribed (SuperScript™ First-Strand Synthesis System for RT-PCR, Thermo Fisher, Waltham, MA) from MCF10A RNA isolated using the RNeasy Plus Mini Kit (Qiagen, Germantown, MD). The obscurin PH-domain cDNA was then PCR amplified (OneTaq 2× Master Mix, NEB, Ipswich, MA), ligated into the pCMV6-Entry Mammalian Expression Vector (OriGene, Rockville, MD) at AscI and XhoI sites (NEB, Ipswich, MA), and transformed into E. coli cells. To eliminate the terminal DDK tag, the obscurin PH-Myc (oPH-Myc) was PCR-amplified from the resultant pCMV6-oPH-Myc-DDK plasmid using OneTaq 2× Master Mix (NEB, Ipswich, MA) and ligated into the pCMV6-Entry Mammalian Expression Vector (OriGene, Rockville, MD) at AscI and PmeI sites (NEB, Ipswich, MA), introducing a stop codon prior to the DDK tag. The pCMV6-oPH-Myc plasmid was subsequently gel purified using the QIAquick Gel Extraction Kit (Qiagen, Germantown, MD) and transformed into E. coli cells. In a subsequent reaction, the Myr tag was inserted via PCR amplification of the oPH-Myc sequence from the pCMV6-oPH-Myc plasmid, using a primer set that contained the Myr sequence and the OneTaq 2× Master Mix (NEB, Ipswich, MA). The obtained amplicon was subsequently ligated into the pCMV6-Entry Mammalian Expression Vector (OriGene, Rockville, MD) at AscI and PmeI sites (NEB, Ipswich, MA), gel purified using the QIAquick Gel Extraction Kit (Qiagen, Germantown, MD) and transformed into E. coli cells.

Kalirin Pleckstrin Homology (PH)-Domain constructs: The kalirin PH-domain sequence was purchased as a custom insert in the pUC57 donor plasmid (GenScript, Piscataway, NJ). The kalirin PH-domain sequence was PCR amplified (OneTaq 2× Master Mix, NEB, Ipswich, MA) from the pUC57 plasmid, ligated into the pCMV6-Entry Mammalian Expression Vector (OriGene, Rockville, MD) at AscI and XhoI sites (NEB, Ipswich, MA), and transformed into E. coli cells. To eliminate the terminal DDK tag, the kalirin PH-Myc (kPH-Myc) sequence was then PCR-amplified from the resultant pCMV6-kPH-Myc-DDK plasmid vector using OneTaq 2× Master Mix (NEB, Ipswich, MA) and ligated into the pCMV6-Entry Mammalian Expression Vector (OriGene, Rockville, MD) at AscI and PmeI sites (NEB, Ipswich, MA), introducing a stop codon prior to the DDK tag. The ligated pCMV6-kPH-Myc plasmid was subsequently gel purified using the QIAquick Gel Extraction Kit (Qiagen, Germantown, MD) and transformed into E. coli cells. In a subsequent reaction, the Myr tag was introduced via PCR amplification of the kPH-Myc sequence from the pCMV6-kPH-Myc plasmid, using a primer set that included the Myr sequence and the OneTaq 2× Master Mix (NEB, Ipswich, MA). The resultant Myr-kPH-Myc amplicon was ligated into the pCMV6-Entry Mammalian Expression Vector (OriGene, Rockville, MD) at AscI and PmeI sites (NEB, Ipswich, MA), gel purified using the QIAquick Gel Extraction Kit (Qiagen, Germantown, MD) and transformed into E. coli cells.

PLCγ1 Pleckstrin Homology (PH)-Domain constructs: The PLCγ1 PH-Myc and PLCγ1 Myr-PH-Myc sequences were purchased as custom inserts in the pUC57 donor plasmid (GenScript, Piscataway, NJ). The PLC γ 1 PH-Myc or PLC γ 1 Myr-PH-Myc sequences were PCR amplified (OneTaq 2× Master Mix, NEB, Ipswich, MA) from the pUC57 plasmid, ligated into the pCMV6-Entry Mammalian Expression Vector (OriGene, Rockville, MD) at AscI and XhoI sites (NEB, Ipswich, MA) introducing a stop codon prior to the DDK tag, gel purified using the QIAquick Gel Extraction Kit (Qiagen, Germantown, MD), and transformed into E. coli cells.

All plasmids were transformed into One Shot TOP10 chemically competent E. coli cells (Thermo Fisher, Waltham, MA). All primer sets are listed in Table 5. The authenticity of all plasmids was verified by Sanger sequencing (Genewiz, South Plainfield, NJ).

### Transient Transfection

MDA-MB-231 cells used for immunoblotting or transwell migration assays were plated at a 3×10⁵ cells/well density in 6-well plates (Corning, Corning, NY), while cells for immunofluorescence experiments were plated at a 3×10⁴ cells/well density in 96-well plates (Ibidi, Fitchburg, Wisconsin). Cells were allowed to adhere for 24 h in a humidified tissue culture incubator at 37° C., 5% CO₂, before they were transiently transfected with the indicated plasmids, using Lipofectamine 3000 Transfection Reagent (Thermo Fisher, Waltham, MA; #L3000008). All transfections were completed in DMEM 10% FBS, 1% penicillin-streptomycin for 24 h. Cells were allowed to recover in complete growth media for an additional 2 or 24 h before further experimentation.

### Adenovirus Generation and Treatments

Myr-Myc and Myr-oPH-Myc adenoviruses were generated by SignaGen Laboratories (Frederick, MD, USA). In brief, flanking BamHI and HindIII sites were added to the Myr-Myc DNA sequence via PCR amplification followed by cloning into the pAD-CMV-RFP shuttle vector. Likewise, EcoRI and PmeI restriction sites were added to the Myr-oPH-Myc DNA via PCR amplification for insertion into the Ad-CMV-RFP shuttle vector. The resultant adenovirus shuttle plasmids were then combined into the pAD backbone and packaged into HEK293 cells, obtained by ATCC (RRID:CVCL_0045), by transfection using the LipoJet™ In Vitro Transfection Kit (Ver. II, #SL100468). Two weeks following transfection, adenoviruses were harvested and further amplified in HEK293 cells. Titration was performed using the Adeno Rapid Titer Kit (632250) from Takara (San Jose, CA).

For adenovirus treatments, MDA-MB-231 and SKBr3 cell monolayers were plated at a 3×10⁵ cells/well density in 6-well plates (Corning, Corning, NY) and allowed to adhere for 24 h in a humidified tissue culture incubator at 37° C., 5% CO₂ before infection with Myr-Myc control or Myr-oPH-Myc adenovirus at a MOI of 400 in complete growth media. SKBr3 cells used specifically for transwell migration assays were plated in 100 mm tissue-culture dishes (Corning, Corning, NY) at a 1.5×10⁶ cells/plate density. Twenty-four h following treatment, media was replaced with fresh, adenovirus-free, complete growth media, while 24 h later cells were either harvested for protein lysates or replated for immunofluorescence, gelatin invasion, transwell migration, single cell migration through microchannels, spheroid invasion, or adhesion assays.

### Lipid Nanoparticle (LNP) Formulation and Cell Treatment

LNPs were formulated using the NanoAssemblr® Ignite platform (Precision NanoSystems, Vancouver, Canada), which utilizes a Y-shaped staggered herringbone micromixer cartridge (Precision NanoSystems, Vancouver, Canada). The lipid phase was composed of Dlin-KC2-DMA (2,2-dilinoleyl-4-dimethylaminoethyl-[1,3]-dioxolane; MedKoo Biosciences Inc., Morrisville, NC), DSPC (1,2-distearoyl-sn-glycero-3-phosphocholine; Avanti Polar Lipids Inc., Alabaster, Alabama), cholesterol (Sigma-Aldrich, St. Louis, MO), and mPEG2000-DSPE (1,2-dimyristoyl-rac-glycero-3-methoxypolyethyleneglycol-2000; Avanti Polar Lipids Inc., Alabaster, Alabama). The lipids were dissolved in ethanol (Koptec) at a molar ratio of KC2:DSPC:cholesterol:mPEG2000-DSPE of 50:10.5:38.1:1.5. The aqueous phase contained the desired DNA segment in water (DNase, RNase and protease tested, sterile filtered; Quality Biological, Gaithersburg, MD). A nitrogen-to-phosphate ratio (N/P) of 8 between the ionizable lipid and DNA was maintained for all formulations to sustain optimal charge balance and packaging of plasmid DNA into LNPs. The aqueous-to-lipid flow rate ratio (FRR) was maintained at 3:1 for the formulation at a flow rate of 12 mL/min. An initial waste volume of 0.45 mL and a final waste volume of 0.05 mL was set for all formulations. LNPs were purified using Amicon Ultra Centrifugal Filters (Millipore Sigma, Burlington, MA). Hydrodynamic size (z-avg), polydispersity index (PDI), and surface charge (zeta potential) were determined by dynamic light scattering (DLS) using Malvern Zetasizer Nano-ZS Zen 3600. Purified LNPs were diluted 100 times in water for hydrodynamic size and PDI assessments, while for zeta potential measurements LNPs were diluted 100 times in PBS. The DNA concentration after purification was measured using a NanoDrop 2000 (Thermo Fisher, Waltham, MA). All LNPs were stored at 4° C. and used within 6 h of formulation.

MDA-MB-231 cell monolayers were plated at a 3×10⁵ cells/well density in 6-well plates (Corning, Corning, NY) for western blot and transwell migration assays, while SKBr3 cell monolayers were plated at a 3×10⁵ cells/well density in 6-well plates (Corning, Corning, NY) for western blot and a 1.5×10⁶ cells/plate density in 100 mm dishes (Corning, Corning, NY) for transwell migration assays. MDA-MB-231 and SKBr3 cells were allowed to adhere for 24 h in a humidified tissue culture incubator at 37° C., 5% CO₂ before treatment with 20 μg/mL and 10 μg/mL, respectively, of LNPs carrying control Myr-Myc, Myr-oPH-Myc, Myr-kPH-Myc or Myr-γPH-Myc plasmid in Opti-MEM Reduced Serum Medium (Thermo Fisher, Waltham, MA). Twenty-four h post-treatment, the LNP-containing Opti-MEM media was replaced with fresh, particle-free complete growth media, and 24 h later, cells were either harvested for protein lysates or replated for transwell migration assays.

### Immunofluorescence, Confocal Microscopy, and Stimulated Emission Depletion (STED) Microscopy

For confocal microscopy, cell monolayers were plated on non-coated or pre-coated with the indicated substrate Polymer Coverslip μ-Plate 96-well Square multiwell plates (Ibidi, Fitchburg, Wisconsin). Following permeabilization with 100% methanol for 15 min at −20° C., cells were washed with 1×PBS, blocked in 3% bovine serum albumin (BSA) for 1 h at room temperature, re-washed with 1×PBS, and incubated with primary antibodies for Myc-tag (mouse monoclonal antibody 9E10; Abcam, Waltham, MA; ab32), PI3K/p85 (rabbit monoclonal antibody 19H8; Cell Signaling Technology, Danvers, MA #4257), RFP (rabbit polyclonal antibody, V22R36; Thermo Fisher, Waltham, MA; PA1-986), and 0-actin (goat polyclonal antibody, LSBio, Lynnwood, WA; LS-B15553) overnight at 4° C. in a 0.3% Triton-X-100, 1% BSA, 1×PBS solution. Following extensive washes with 1×PBS, cells were treated with the appropriate secondary antibodies: anti-mouse AlexaFluor 488 (Thermo Fisher, Waltham, MA), anti-rabbit AlexaFluor 568 (Thermo Fisher, Waltham, MA), and anti-goat Alexa Fluor 633 (Thermo Fisher, Waltham, MA) at room temperature in the dark for 1 h. Samples were subsequently stained with DAPI (Thermo Fisher, Waltham, MA), immersed in Ibidi Immersion Oil (Ibidi, Fitchburg, Wisconsin), and stored at 4° C. in the dark until imaging. Images were acquired on a Nikon W1 spinning disk with a 60× oil immersion objective, with pixel sizes at 0.06 or 0.11 μm/pixel. Line composite graphs of Myc-Tag and p85 colocalization were generated via fluorescence pixel intensity (gray value) measurements acquired with ImageJ software (National Institutes of Health, Bethesda, Maryland), where a straight line was drawn at random across the cell body and pixel intensity of the Myc-Tag and p85 channels (mean gray value) versus distance (μm) was plotted.

Spheroids were stained as previously described (V. Padmanaban et al., Nat Protoc, (2020), 15:2413). Briefly, following fixation in 4% PFA for 15 min at room temperature, spheroids were washed in 1× Dulbecco's phosphate buffered saline (D-PBS), permeabilized with 0.5% Triton-X-100 in 1×D-PBS for 1 h at room temperature, and blocked with 10% FBS, 1% BSA in 1×D-PBS for 4 h at room temperature. Spheroids were subsequently incubated with primary antibodies against Myc-tag (mouse monoclonal antibody 9E10; Abcam, Waltham, MA; ab32) and MMP-2 (goat polyclonal antibody I34C660; R&D systems, Minneapolis, MN; AF902) in 1% FBS, 1% BSA, and 0.2% Triton-X-100 in 1×D-PBS overnight at 4° C. Following extensive washes in 1×D-PBS, spheroids were incubated at room temperature in the dark for 4 h with the appropriate secondary antibodies: anti-mouse AlexaFluor 488 (Thermo Fisher, Waltham, MA) and anti-goat AlexaFluor 594 (Thermo Fisher, Waltham, MA) in 1% FBS, 1% BSA, and 0.2% Triton-X-100 in 1×D-PBS. All spheroids were subsequently counter-stained with Alexa Fluor 647 Phalloidin (Cell Signaling Technology, Danvers, MA; #8940) for 15 min at room temperature in the dark, followed by DAPI (Thermo Fisher, Waltham, MA) for 10 min at room temperature in the dark, immersed in Ibidi Immersion Oil (Ibidi, Fitchburg, Wisconsin), and stored in 4° C. in the dark for a maximum of 48 h before imaging. Images were acquired on a Nikon W1 spinning disk with a 20× air objective, with pixel sizes at 0.32 μm/pixel. Whole spheroid MMP2 expression was measured using ImageJ software (National Institutes of Health, Bethesda, Maryland), where a region of interest (ROI) was first drawn around each spheroid edge using β-actin staining as reference, followed by measuring MMP2 fluorescence pixel intensity (mean gray value).

For stimulated emission depletion (STED) microscopy, cell monolayers were plated on fibronectin pre-coated 1.5H Glass Coverslip μ-Plate 96-well Square multiwell plates (Ibidi, Fitchburg, Wisconsin). Following permeabilization with 100% methanol for 15 min at −20° C., cells were washed with 1×D-PBS, blocked in 3% BSA for 1 h at room temperature, re-washed with 1×D-PBS, and incubated with primary antibodies for Myc-tag (mouse monoclonal antibody 9E10; Abcam, Waltham, MA; ab32) and PI3K/P85 (rabbit monoclonal antibody 19H8; Cell Signaling Technology, Danvers, MA #4257) overnight at 4° C. in a 0.3% Triton-X-100, 1% BSA, 1×PBS solution. Following extensive washes with 1×D-PBS, cells were treated with the appropriate secondary antibodies: anti-mouse Abberior Star Orange (Abberior, Gottingen, Germany) and anti-rabbit Abberior Star Red (Abberior, Gottingen, Germany) at room temperature in the dark for 1 h. Samples were subsequently immersed in Abberior Mount Liquid AntiFade (Abberior, Gottingen, Germany), and stored at 4° C. in the dark until imaging. STED images were acquired with the Abberior Facility Line STED microscope using an Olympus 60×/1.42 STED-UPLXAPO60xO oil immersion lens. Abberior STAR Orange and STAR Red were excited at 561 nm and 640 nm, respectively, and depletion was performed with a 775 nm pulsed laser with a gating of 1-8 ns and a pixel dwell time of 5 μs. The emission from both channels was collected with the matrix detector, and the images were post-processed with the Abberior Inspector software. Each line was scanned with 20-line accumulations during acquisition. The pixel size was set to 30 nm, and the pinhole was set to 1 AU.

### Stimulated Emission Depletion (STED) Microscopy Analysis

For all STED analysis, matrix post-processed images (Abberior Inspector software) were evaluated to calculate percent (%) p85 sequestration per cell using ImageJ software (National Institutes of Health, Bethesda, Maryland). All images were initially converted to TIFF files, equally adjusted for brightness and contrast per secondary antibody controls and converted to binary images. ROIs were then drawn around the cell membrane and extraneous extracellular signal was eliminated. To visualize p85 signal that solely localized with Myc-Tag signal, the p85 binary image was overlayed with the Myc-Tag binary image (“AND” operation) using the Image Calculator feature. Mean gray values of the resultant p85/Myc-Tag overlay and the individual p85 binary image were measured, and percent (%) p85 sequestration per cell was calculated as:

$$Percent p 85 Sequestration per Cell = \frac{p 85 AND Myc - Tag}{p 85} \times 100$$

Per PH-domain construct, 15-18 cells were imaged, with 5-8 cells per biological replicate, from three biological replicates per construct. Percent (%) p85 sequestration per cell was then plotted per PH-domain construct.

### Generation of Protein Lysates and Western Blotting

Protein lysates from cell monolayers were generated as follows: media was collected and stored on ice, while cells were gently washed with 1×PBS. The media fraction and the PBS wash were combined and centrifuged at 1750 rpm for 5 min at 22° C. to isolate weakly adherent cells; weakly adherent cell pellets were stored on ice. The remaining cell monolayer was scraped into radioimmunoprecipitation assay buffer (RIPA, Sigma, St. Louis, MO), supplemented with Halt protease and phosphatase inhibitors (Thermo Fisher, Waltham, MA), and then combined with the weakly adherent cell pellet.

Protein lysates from primary tumors were generated as follows: tumors were flash frozen in liquid nitrogen, followed by vigorous tissue homogenization using mortar and pestle. Samples were then dissolved in RIPA buffer (Sigma, St. Louis, MO), supplemented with Halt protease and phosphatase inhibitors (Thermo Fisher, Waltham, MA). All samples collected in RIPA buffer were then lysed via rotation for 30 min at 4° C., followed by centrifugation at 20,000 g for 10 min at 4° C. to isolate the protein fraction from cell debris.

Protein lysate concentration was determined using the Quick Start Bradford Protein Assay (Bio-Rad, Hercules, CA), and 15-20 μg of protein lysates were separated using either NuPAGE 3-8% Tris-acetate SDS-PAGE gels (specifically for Western blots of obscurin expression) or NuPAGE 4-12% Bis-Tris gels (all other proteins). Proteins were transferred to nitrocellulose membranes for immunoblotting. All membranes were blocked for 2 h in 3% BSA in Tris Buffered Saline (TBS) with 0.1% Tween-20 (TBST), before overnight incubation at 4° C. with primary antibodies to obscurin-Ig58/59 (rabbit polyclonal; 0.5 μg/mL (T. Guardia et al., Proc Natl Acad Sci USA, (2023), 120:e2215553120)), HSP90 (C45G5, rabbit monoclonal antibody, Cell Signaling Technology, Danvers, MA; #4877), Myc-Tag (71D10, rabbit monoclonal antibody; Cell Signaling Technology, Danvers, MA; #2278), pan-Akt (C67E7, rabbit monoclonal antibody; Cell Signaling Technology, Danvers, MA; #4691), GAPDH-71.1 (mouse monoclonal antibody; Sigma, St. Louis, MO; G8795), PI3K/p85 (19H8, rabbit monoclonal antibody; Cell Signaling Technology, Danvers, MA; #4257), phospho-PI3K/P85-Tyr458/p55-Tyr199 (rabbit monoclonal antibody; Cell Signaling Technology, Danvers, MA; #4228), phospho-Akt-Thr308 (C31E5E, rabbit monoclonal antibody; Cell Signaling Technology, Danvers, MA; #2965), phospho-Akt-Ser473 (D9E, XP rabbit monoclonal antibody; Cell Signaling Technology, Danvers, MA; #4060), PARP (46D11, Rabbit monoclonal antibody; Cell Signaling Technology, Danvers, MA; #9532); Cleaved PARP (Asp214) (D64E10, XP Rabbit monoclonal antibody; Cell Signaling Technology, Danvers, MA; #5626), MMP-1 (E9S9N, rabbit monoclonal antibody; Cell Signaling Technology, Danvers, MA; #54376), MMP-2 (D4M2N, rabbit monoclonal antibody; Cell Signaling Technology, Danvers, MA; #40994), and MMP-9 (D603H, XP Rabbit monoclonal antibody; Cell Signaling Technology, Danvers, MA; #13667) in 3% BSA TBST overnight at 4° C. Following extensive washes with TBST, membranes were incubated with horseradish peroxidase (HRP) conjugated anti-rabbit or anti-mouse secondary antibody (Cell Signaling Technology, Danvers, MA). Immunoreactive bands were visualized either with Pierce ECL Western Blotting Substrate (Thermo Fisher, Waltham, MA) or SignalFire ECL Reagent (Cell Signaling Technology, Danvers, MA) kits. Densitometric evaluation was performed using ImageJ software (National Institutes of Health, Bethesda, Maryland). Three to seven biological replicates were performed for each experiment. All blots are presented in grayscale mode.

### Wound Healing Assay

MDA-MB-231 and SKBr3 cells were plated at a 3×10⁵ cells/well density in 6-well plates. MDA-MB-231 cells were allowed to adhere for 24 h before they were treated with Myr-Myc control or Myr-oPH-Myc adenovirus (MOI 400) in complete growth media for 24 h. Cells were supplied with fresh growth media for 24 h, and then a scratch was performed. SKBr3 cells were allowed 6 days to reach confluence following plating, after which one well was trypsinized to obtain a final cell count, while the other wells were treated with Myr-Myc control or Myr-oPH-Myc adenovirus (MOI 400) in complete growth media. Twenty-four h following adenovirus treatment, cells were supplied with fresh growth media for an additional 24 h, and then a scratch was performed.

All scratches were generated with a 200 μL sterile pipette tip and cells were subsequently washed with 1×PBS. Following the scratch/wound, cells were cultured in complete growth media for 42 h (MDA-MB-231) and 96 h (SKBr3) in a humidified tissue culture incubator with 5% CO₂ at 37° C. Images were taken at 0, 16, 24, and 42 h (MDA-MB-231) or 0, 24, 45, and 96 h (SKBr3) using the EVOS FL cell imaging system (Thermo Fisher, Waltham, MA) under a 4× objective. Collective cell migration was quantified as percent (%) wound closure, where cell edge boundaries were drawn manually and quantified using ImageJ software (National Institutes of Health, Bethesda, Maryland); five to six biological replicates were performed per cell line.

### Transwell Migration Assay

Transiently transfected, adenovirus-infected, and LNP-treated MDA-MB-231 or SKBr3 trypsinized cells were plated at a density of 5×10⁴ or 1×10⁶ cells, respectively, in 500 μL of serum-free medium onto a 24-well impermeable insert upper chamber containing 8.0 μm sized pores (Corning, Corning, NY). The lower chamber of the wells was filled with complete growth media containing 10% FBS. Chambers were incubated for 24 h (MDA-MB-231) or 48 h (SKBr3) in a humidified tissue culture incubator with 5% CO₂ at 37° C. Non-migratory cells on the upper surface were removed using a cotton swab, while migratory cells on the lower surface were stained using a fixative and two stain solutions (Modified Giemsa) supplied by the Differential Quick Stain Kit (Polysciences, Inc., Warrington, PA) for 5 min per solution, followed by extensive washing in distilled water between solution stains. Membranes were then removed from the insert and placed on slides for imaging. Migrating cells were quantified by counting 3 random fields from three to four independent experiments under an inverted light microscope (Olympus IX51, Center Valley, PA; 10× objective).

### Microfluidic Device Fabrication, Cell Seeding, Live-Cell Imaging and Analysis

Adenoviral-treated MDA-MB-231 cells were selected for construct expression using complete growth media supplemented with 500 μg/mL Geneticin G418 (Life Technologies) for 4-7 days, with fresh Geneticin G418 complete growth media being re-supplied every 72 h. Of note, parental SKBr3 cells displayed minimal single cell migration through confined microchannels, as previously reported; therefore, they could not be reliably tested. PDMS-based microfluidic devices containing a parallel series of microchannels of 10 μm in height, 3 μm in width, and 200 μm in length were fabricated as previously described (C. L. Yankaskas et al., Sci Adv, (2021), 7 (28); R. Zhao et al., Sci Adv 2021, 7 (17)). All microchannel dimensions were routinely confirmed by laser profilometer and then incubated with rat tail collagen I (20 μg/mL, Thermo Fisher, Waltham, MA) for 1-2 h at 37° C. in 5% CO₂ prior to migration assays. Migration assays were performed in DMEM containing 10% FBS and 1% penicillin-streptomycin supplemented with 500 μg/mL Geneticin G418. No chemotactic stimulus was provided. For each device, 20 μL of 5×10⁶ cells/mL cell suspension in Geneticin G418 complete growth media was added to the inlet well. Prior to migration experiments, fresh Geneticin G418-containing growth media was added to the inlet and outlet wells. Timelapse images were subsequently recorded in 10 min intervals for up to 24 h under an inverted Nikon Eclipse Ti microscope (Nikon, Tokyo, Japan) equipped with a stage-top incubator (Okolab, Pozzuoli, Italy, or Tokai Hit, Shizuoka, Japan) at 37° C. in 5% CO₂, automated controls (NIS-Elements, Nikon), and 10×/0.45 numerical aperture Ph1 objective. Cell migration analysis was performed as previously described (C. L. Yankaskas et al., Sci Adv, (2021), 7 (28); R. Zhao et al., Sci Adv 2021, 7 (17)). Live cell migration videos were exported to ImageJ (National Institutes of Health, Bethesda, Maryland). Cell entry time and percent cell entry were calculated manually, where cell entry time was defined as the time interval from the time point the leading cell edge initiated entry into a microchannel until the entire cell had fully entered. All tracks of fully entered individual cells were obtained using the MTrackJ plugin (Biomedical Imaging Group Rotterdam, Erasmus University Medical Center, Netherlands). Cell migration speed, velocity, and persistence were subsequently calculated from these tracks using a custom MATLAB script (MathWorks, Natick MA). Experiments were repeated three independent times.

### Adhesion Assays

96-well plates were precoated with ECM proteins as previously described (F. Baltes et al., Biochim Biophys Acta Mol Cell Res, (2020), 1867:118663). In brief, plates were incubated for 24 h in a humidified tissue culture incubator at 37° C., 5% CO2 with 6 μg/mL of either human fibronectin, collagen I, or laminin (Corning, Corning, NY), dissolved in 1×PBS. Control wells for adhesion to plastic were precoated with equal volumes of 1×PBS. Matrix-coated wells were blocked with 3% BSA in 1×PBS for 1 h at 37° C., 5% CO2 in a humidified tissue culture incubator. Adenovirus-treated MDA-MB-231 and SKBr3 cells were plated in each well at a 3×10⁴ cells/well density and incubated in a humidified tissue culture incubator at 37° C., 5% CO2 for 30 min and 2 h, respectively. Immediately following adhesion, wells were washed 3 times with 1×PBS, fixed with 100% methanol at −20° C. for 15 min, and stained with DAPI. Plates were subsequently stored in the dark at 4° C. and imaged within one week using the EVOS FL cell imaging system (Thermo Fisher, Waltham, MA) under a 4× objective. Adherent cells were quantified by summing DAPI positive particles present per field from three random fields per well, from four to six biological replicates using ImageJ software (National Institutes of Health, Bethesda, Maryland).

### Filopodia Analysis

Following staining for RFP, Myc-tag, and β-actin, as well as image acquisition of Myr-Myc and Myr-oPH-Myc adenovirally-transduced single cells, cell filopodia were counted and their length measured using the ImageJ Single Image FiloQuant plugin (Turku, Finland). In short, ten, adenovirus-treated, RFP-positive cells per construct (i.e., Myr-Myc and Myr-oPH-Myc) were selected at random per biological replicate for analysis (n=5 biological replicates per ECM matrix substrate). To visualize filopodia, cell images for β-actin staining were contrast-adjusted and cropped to include only the selected cell edges (containing filopodia), while surrounding RFP-negative cells were cleared from the image. Processed cell images were converted to TIFF files. FiloQuant settings (edge detection, filopodia detection, and contour detection thresholds) per TIFF file were custom-adjusted to highlight cell edge protrusions and exclude false positive hits, as outlined by the FiloQuant Manual V1.0 (https://imagej.net/media/filoquant-manual-v1.pdf).

### Bradykynin Treatment

Cells were treated with 100 ng/ml bradykinin (Tocris, Bristol, UK #3004) in all assays. For imaging of filopodia via β-actin fluorescence, 10 min prior to fixation with 100% methanol, bradykinin-containing growth media was added to cells while incubated at 37° C., 5% CO₂ in a humidified tissue culture incubator. For transwell migration assays, cells were plated in bradykinin-containing serum-free media in the upper chamber, whereas the lower chamber contained 100 ng/mL bradykinin in growth media. For adhesion assays, adenovirus-treated cells were plated in bradykinin-containing growth media and allowed to adhere for 30 min at 37° C., 5% CO₂ in a humidified tissue culture incubator, before being further processed for staining and visualization.

### Spheroid Formation and 3D Collagen Invasion Assay

Spheroid experiments were performed using either adenovirus pre-treated or untreated MDA-MB-231 cells, as previously described (S. Dadakhujaev et al., Oncoscience, (2014), 1 (3), 229). Parental SKBr3 cells were unable to form spheroids, likely due to their weak(er) metastatic potential compared to MDA-MB-231 cells (I. B. Juan Manuel Iglesias et al., PLoS One, (2013), 8), and therefore were not tested. 3×10³ MDA-MB-231 cells pre-treated with Myr-Myc (MOI:400) or Myr-oPH-Myc (MOI:400) adenovirus were suspended in 150 μL of ice-cold Growth Factor Reduced Matrigel (Corning, Corning, NY), which was generated by diluting DMEM containing 10% FBS and 1% penicillin-streptomycin at 1:50 ratio Matrigel to DMEM media. The cell-Matrigel suspension was then gently plated in different wells followed by incubation at 37° C., 5% CO₂ in a cell culture incubator for 48 h before embedding for invasion assays. For spheroids treated with LNPs, 3×10³ untreated MDA-MB-231 cells were suspended in the same Matrigel-DMEM media and allowed to form spheroids for 24 h, before addition of either Myr-Myc, Myr-oPH-Myc, Myr-kPH-Myc, or Myr-γPH-Myc LNPs directly into each well at 20 μg/mL. LNP-treated spheroids were then allowed to grow for an additional 24 h before embedding for invasion assays.

3D collagen invasion assays using spheroids were performed as previously described⁷. In short, 3 ml of rat tail collagen type I (Corning, Corning, NY) were mixed with 375 μl of 10×DMEM-low glucose (Sigma, St. Louis, MO). The pH of the mixture was gradually adjusted to physiological levels with NaOH. 30 μl of the mixture were then added to a Falcon 24 well-plate (Corning, Corning, NY) and incubated at 37° C., 5% CO₂ in a cell culture incubator for ~20 min. Spheroids were collected into 1.5 ml Eppendorf tubes by disrupting the Matrigel gently and incubated on ice for >10 min to further depolymerize the Matrigel. Spheroids were isolated by centrifugation (1500 rpm) for 6 sec and resuspended into 240 μl of the pH-balanced collagen mixture. Next, 120 μl of the spheroid-collagen mixture were plated in each well and incubated at 37° C., 5% CO₂ in a cell culture incubator for 1 h. Following collagen polymerization, 1 ml cell culture media was added to each well. Time-lapse images were recorded in 20 min intervals for 16-48 h under an inverted Nikon Eclipse Ti microscope (Nikon) equipped with a stage-top incubator (Okolab or Tokai Hit) at 37° C. and 5% CO2, automated controls (NIS-Elements, Nikon) and a 10×/0.45 numerical aperture Ph1 objective. Cell tracks were obtained using the MTrackJ plugin (Biomedical Imaging Group Rotterdam, Erasmus University Medical Center, Netherlands), and resultant cell velocity, speed, and mean square displacement (MSD) were calculated using a custom-made MATLAB script. Spheroid area size and normalized area of expansion were measured manually using ImageJ software (National Institutes of Health, Bethesda, Maryland). Data was acquired from three independent experiments.

### Gelatin Invasion Assay, Invadopodia Quantification, and MMP Staining

96-well plates (Ibidi, Fitchburg, Wisconsin) were coated with Fluorescein-Gelatin using the QCM Gelatin Invadopodia Assay Kit (Green) (Millipore Sigma, Burlington, MA; ECM670). Following aldehyde quenching, MDA-MB-231 and SKBr3 cells treated with Myr-Myc (MOI:400) or Myr-oPH-Myc (MOI:400) adenovirus were seeded at a 3×10⁴ cells/well density onto fresh Fluorescein-Gelatin-coated plates and allowed to invade for 24 h (MDA-MB-231) or 72 h (SKBr3). Cells were subsequently fixed in 3.7% paraformaldehyde (PFA), 1×D-PBS solution for 30 min in the dark, washed with 1×D-PBS, and stored in the dark at 4° C. for no longer than one week.

To preserve fluorescence, all further steps were performed in the dark. To stain invadopodia and MMPs, fixed cells were first permeabilized with 0.3% Triton-X-100 for 5 min at room temperature, blocked with 3% BSA for 60 min at room temperature, and then incubated with primary antibodies against Myc-Tag (mouse monoclonal antibody 9E10; Abcam, Waltham, MA; ab32), MMP-2 (rabbit monoclonal antibody D4M2N; Cell Signaling Technology, Danvers, MA; #40994), or MMP-9 (XP rabbit monoclonal ab D603H; Cell Signaling Technology, Danvers, MA; #13667) in 2% BSA, 0.25% Triton-X-100 overnight at 4° C. Cells were washed with 1×D-PBS and incubated with the appropriate secondary antibodies, anti-mouse AlexaFluor 405 (Thermo Fisher, Waltham, MA) and anti-rabbit AlexaFluor 594 (Thermo Fisher, Waltham, MA), and counterstained with AlexaFluor 647 Phalloidin (Cell Signaling Technology, Danvers, MA; #8940) in 2% BSA, 0.25% Triton-X-100 for 1 h at room temperature. Cells were subsequently washed 2× with 2% BSA, 0.25% Triton-X-100 and 3× with 1×D-PBS, before they were immersed in Ibidi Immersion Oil (Ibidi, Fitchburg, Wisconsin) and stored at 4° C. in the dark.

All images were acquired using a Nikon W1 spinning disk with a 60× oil immersion objective, with pixel sizes at 0.11 μm/pixel. For image presentation, channels were pseudo-colored using ImageJ software (National Institutes of Health, Bethesda, Maryland) as follows: gelatin (red), RFP (gold), Myc-tag (Cyan), Phalloidin/F-actin (green), and MMP2/9 (magenta). To visualize gelatin degradation and invadopodia, the imaging plane was centered at the cell-gelatin interface. To acquire images of MMP expression, the imaging plane was centered above the gelatin plane at the cell body using phalloidin/F-actin staining as reference. Gelatin degradation was quantified as 1/Mean Gray Value of fluorescein fluorescence from 10 randomly selected, RFP-positive cells per biological replica for each construct (n=3 biological replicates, 30 cells per construct) using ImageJ software (National Institutes of Health, Bethesda, Maryland). Fluoresceine gelatin and Phalloidin/F-actin channels were input into the ImageJ JaCoP plugin (National Institutes of Health, Bethesda, Maryland) and invadopodia were quantified as 1-Mander's Coefficient for the fraction of phalloidin/F-actin signal that overlaps with gelatin signal. MMP expression was quantified from 20 randomly selected, RFP-positive cells per biological replica for each construct (n=3 biological replicates, 60 cells per construct), as the mean gray value of MMP fluorescence per cell, using ImageJ software (National Institutes of Health, Bethesda, Maryland).

### Animal Studies

All animal studies were performed following the Institutional Animal Care and Use Committee (IACUC) procedures and guidelines of the University of Maryland, Baltimore under an approved protocol. Eight-to-twelve-week-old female NOD SCID mice weighing 19-25 g were obtained from Charles River (Fredrick, MD) and fed ad libitum. Prior to injection, animals were randomly assigned to the Myr-Myc or Myr-oPH-Myc LNP group. At Day 0, 1×10⁶ MDA-MB-231 cells were suspended in 100 μL PBS and mixed with equal volume of Matrigel (Corning). Cell number was quantified via Countess® Automated Cell Counter (ThermoFisher). The cell suspension was injected subcutaneously into the fourth mammary gland on the ventral surface of the abdomen of each female mouse. Following tumor implantation, 10 μg Myr-Myc or Myr-oPH-Myc LNPs were injected into primary tumors that had grown to ~100 mm³ or into day-7 tumor-bearing mouse mammary fat pads, once a week for 6 weeks. Tumor volumes were measured with an external digital caliper (Thomas Scientific, Inc.) weekly until endpoint, along the two longest perpendicular axes in the x/y plane to the nearest 0.1 mm. Depth was assumed to be equivalent to the shortest of the perpendicular axes (y), and volume was calculated according to the: V=xy²/2 formula, as the standard practice for xenograft tumors.

hLINE qPCR Analysis of Metastatic Burden

DNA was extracted from frozen lung, axillary lymph node, liver, bone, and brain specimens using the DNeasy Blood and Tissue kit (Qiagen), according to the manufacturer's recommendations. For bone, surrounding muscle tissue was dissected followed by flash-freezing of the entire femur in liquid nitrogen, and vigorous homogenization using mortar and pestle until all bone was reduced to powder. For lung, axillary lymph node, liver, and brain, 10-15 mg per piece was collected from each harvested organ, weighed, and recorded. Samples were lysed overnight at 56° C., and purification steps were followed as outlined in the manufacturer's protocol. DNA was eluted in 100 μl (lungs and liver) or 50 μl (axillary lymph node, bone, and brain) of DNA-RNA free water. Quantification of hLINE levels, which serve as proxy for the amount of human DNA present in mouse organs, was performed with qPCR, as previously reported, in a 20 μl reaction with the following components: 10 μl iTaq Universal SYBR Green Supermix (Bio-Rad), 1.5 μl of each 1 μM forward and reverse primers, 4.5 μl purified DNA and 2.5 μl water (T. Guardia et al., Proc Natl Acad Sci USA, (2023), 120:e2215553120). In each qPCR experiment, serial dilutions of human DNA extracted from MDA-MB-231 cells using the DNeasy Blood and Tissue kit (Qiagen) were included to serve as standards. Results are shown as the amount of human DNA per mg of digested tissue sample. Tissues from nine animals per group were analyzed and three technical qPCR replicates were performed for each biological replicate; the hLINE qPCR primer sequences are provided in Table 6.

TABLE 6
Primers for qPCR and RT-qPCR of Mouse Tissues

| | Forward or | |
|---|---|---|
| | Reverse | |
| Primer | Primer | Primer sequence (5'>3') |
| Myr-Myc | Forward | CAAGCCAAAGCTCGAGCAGA |
| | | (SEQ ID NO: 31) |
| Myr-Myc | Reverse | CTTGCCCCTTGCTCCATACC (SEQ |
| | | ID NO: 32) |
| Myr-oPH- | Forward | AGCAAGCCAAAGCTCATGGA |
| Myc | | (SEQ ID NO: 33) |
| Myr-oPH- | Reverse | AGATTACCAGGTGGTTGCGG |
| Myc | | (SEQ ID NO: 34) |
| hLINE | Forward | TCACTCAAAGCCGCTCAACTA |
| | | (SEQ ID NO: 35) |
| hLINE | Reverse | TCTGCCTTCATTTCGTTATGTACC |
| | | (SEQ ID NO: 36) |
| GAPDH | Forward | TCGGAGTCAACGGATTTG (SEQ |
| | | ID NO: 37) |
| GAPDH | Reverse | CAACAATATCCACTTTACCAGAG |
| | | (SEQ ID NO: 38) |

Primary Tumor qPCR Analysis of LNP-Delivered Myr-Myc and Myr-oPH-Myc pDNA

DNA was extracted from frozen primary tumor specimens using the DNeasy Blood and Tissue kit (Qiagen) according to the manufacturer's recommendations. For each tumor, 10-15 mg per piece was collected, weighed, and recorded. Samples were lysed overnight at 56° C., and purification steps were followed as outlined in the manufacturer's protocol. DNA was eluted in 100 μl of DNA-RNA free water. Quantification of Myr-Myc or Myr-oPH-Myc pDNA levels, which serve as confirmation of successful LNP delivery of each pDNA construct, was performed with qPCR as previously reported (T. Guardia et al., Proc Natl Acad Sci USA, (2023), 120:e2215553120). Briefly, qPCR was performed in a 20 μl reaction with the following components: 10 μl iTaq Universal SYBR Green Supermix (Bio-Rad), 1.5 μl of each 1 μM forward and reverse primers, 4.5 μl purified DNA and 2.5 μl water. In each qPCR experiment, serial dilutions of Myr-Myc or Myr-oPH-Myc plasmid DNA extracted from Myr-Myc or Myr-oPH-Myc expressing MDA-MB-231 cells using the DNeasy Blood and Tissue kit (Qiagen) were included to generate standard curves of each pDNA construct. Results are shown as the amount of Myr-Myc or Myr-oPH-Myc pDNA per mg of digested primary tumor sample. Tumors from nine animals per group were analyzed and three technical qPCR replicates were performed for each biological replicate; the Myr-Myc and Myr-oPH-Myc qPCR primer sequences are provided in Table 6.

### Primary Tumor RNA Extraction and RT-qPCR

Total RNA was extracted from primary tumors using the Qiagen RNeasy Plus Mini Kit with gDNA eliminator columns (Qiagen, Germantown, MD), which selectively and efficiently remove genomic DNA. Tumors were flash frozen in liquid nitrogen, followed by vigorous homogenization using mortar and pestle until all tissue was reduced to powder and subsequently processed for RNA extraction. cDNA was synthesized from ~700 ng of total RNA using the SuperScript III First-Strand Synthesis System (Invitrogen, Thermo Fisher). KiCqStart Universal SYBR Green qPCR ReadyMix (Sigma, St. Louis, MO) was used for qPCR reactions. RNA expression levels were normalized to GAPDH using the DDCt method. Three technical qPCR replicates for each of the nine primary tumors were performed; the Myr-Myc and Myr-oPH-Myc qPCR primer sequences are provided in Table 6.

### Immunohistochemistry and Pathology

Animals that reached the designated endpoint (i.e., tumors that received up to 6 injections of either Myr-Myc or Myr-oPH-Myc LNPs) were sacrificed. The lungs, liver, and bone were removed, fixed in formalin for 24 h, embedded in paraffin wax, and serially sectioned (4 μm thick). All immunohistochemistry Human Mitochondria and H&E staining was performed by HistoWiz (Histowiz.com; Brooklyn, NY). A pathology report was performed by a Histowiz senior board-certified research pathologist, where whole slide images, and six regions of interest per slide, were blindly reviewed in their entirety, with all samples designated as experimental.

### Alpha-Fold, In Silico Mutagenesis, and Molecular Dynamics Simulations

All models were generated using alpha-fold and assessed for quality using per-residue local difference distance test (pLDDT) and root mean square deviation (RMSD) across multiple models (J. Jumper et al., Nature, (2021), 596:583). Models were equilibrated for >50 ns in explicit solvent at 37° C., 150 mM NaCl using the Amber14 force field in the program YASARA (H. Land et al., Methods Mol Biol, (2018), 1685:43). All mutations were generated from the equilibrated models by using the ‘swap’ command and then further equilibrated for 10 ns. Simulations were surveyed every 100 psec and analyzed using standard YASARA macros as previously described (H. Land et al., Methods Mol Biol, (2018), 1685:43). All data were visualized in PyMOL (The PyMOL Molecular Graphics System, Version 2.5.5 Schrödinger, LLC.).

### Quantification and Statistical Analysis

All data are presented as the mean±SD or SEM from three or more independent experiments. All images are included as representative examples of the quantitative average per treatment group. Gaussian or non-gaussian distributions were confirmed using the Kolmogorov-Smirnov test for normal distributions. Data sets with Gaussian distributions were compared using two-tailed Student's t-test or one-way ANOVA followed by Tukey's, Dunnett's, or Fisher's LSD multiple comparisons test, as appropriate. For non-Gaussian distributions, the nonparametric Mann-Whitney or Kruskal-Wallis test was used for comparisons between two or more groups, respectively. Two-way ANOVA followed by Dunn's or Sidak multiple comparisons test was used for comparisons between multiple groups with two independent variables. Statistical significance was defined as p<0.05. Calculations were performed using GraphPad Prism 9 or 10 (GraphPad Software). Specific details of statistical analysis per experiment are included in the figure legends.

1. A pharmaceutical composition comprising an effective amount of nucleic acid encoding a polypeptide comprising a pleckstrin homology (PH) domain, a variant or fragment thereof, wherein the fragment of the PH domain comprises a p85-SH3 interacting region (PSIR).

2. The pharmaceutical composition of claim 1, wherein the nucleic acid is RNA.

3. The pharmaceutical composition of claim 1, wherein the nucleic acid is DNA.

4. The pharmaceutical composition of claim 1, wherein the polypeptide comprises a myristoylation tag.

5. The pharmaceutical composition of claim 4, wherein the myristoylation tag is located at the N-terminus of the polypeptide.

6. The pharmaceutical composition of claim 4, wherein the myristoylation tag comprises an amino acid sequence comprising SEQ ID NO:7.

7. The pharmaceutical composition of claim 1, wherein the polypeptide consists essentially of a myristoylation tag and a pleckstrin homology (PH) domain, a variant or fragment thereof, wherein the fragment of the PH domain comprises a p85-SH3 interacting region (PSIR).

8. The pharmaceutical composition of claim 1, wherein the pleckstrin homology (PH) domain is from a human protein.

9. The pharmaceutical composition of claim 1, wherein the pleckstrin homology (PH) domain comprises any one of SEQ ID NOS:1, 3 or 5.

10. The pharmaceutical composition of claim 1, wherein the pleckstrin homology (PH) domain is at least 90% identical to any one of SEQ ID NOS: 1, 3 or 5.

11. The pharmaceutical composition of claim 1, wherein the pleckstrin homology (PH) domain is from an obscurin protein.

12. The pharmaceutical composition of claim 1, wherein the pleckstrin homology (PH) domain is from a kalirin protein.

13. The pharmaceutical composition of claim 1, wherein the pleckstrin homology (PH) domain is from a phospholipase-Cγ1 (PLCγ1).

14. The pharmaceutical composition of claim 1, wherein the nucleic acid is encoded by a viral vector.

15. The pharmaceutical composition of claim 14, wherein the viral vector is selected from an adeno-associated viral vector, an adenoviral vector, a lentiviral vector, and a retroviral vector.

16. The pharmaceutical composition of claim 1, wherein the composition comprises lipid nanoparticles that encapsulate the nucleic acid.

17. The pharmaceutical composition of claim 16, wherein the lipid nanoparticles comprise Dlin-KC2-DMA (2,2-dilinoleyl-4-dimethylaminoethyl-[1,3]-dioxolane), DSPC (1,2-distearoyl-sn-glycero-3-phosphocholine), cholesterol, and mPEG2000-DSPE (1,2-dimyristoyl-rac-glycero-3-methoxypolyethyleneglycol-2000.

18. The pharmaceutical composition of claim 17, wherein the molar ratio of KC2:DSPC:cholesterol:mPEG2000-DSPE ranges from 30-50:5-15:25-40:0.5-5.

19. The pharmaceutical composition of claim 17, wherein the molar ratio of KC2:DSPC:cholesterol:mPEG2000-DSPE is about 50:10.5:38.1:1.5.

20. The pharmaceutical composition of claim 17, wherein the lipid nanoparticles have a nitrogen-to-phosphate ratio (N/P) of between 5-10.

21. The pharmaceutical composition of claim 17, wherein the lipid nanoparticles have a nitrogen-to-phosphate ratio (N/P) of about 8.

22. The pharmaceutical composition of claim 1, wherein the nucleic acid encodes a polypeptide that interacts with the PI3K-p85 regulatory subunit.

23. A method of treating cancer in a subject, comprising administering to the subject an effective amount of the pharmaceutical composition of claim 1.

24. The method of claim 23, wherein the cancer is breast cancer.

25. The method of claim 24, wherein the breast cancer is metastatic breast cancer.

26. The method of claim 23, wherein the administering suppresses PI3K/Akt activity in cancer cells as evidenced by reduced phosphorylation levels of activating p85-Tyr458 as well as Akt-Thr308 and -Ser473.

27. The method of claim 23, wherein the administering inhibits formation of filopodia in cancer cells.

28. The method of claim 23, wherein the administering inhibits migration and adhesion of cancer cells to pre-metastatic niche extracellular matrix substrates.

29. The method of claim 23, wherein the administering inhibits invadopodia and matrix metalloproteinase expression.
